SEQUENCE LISTING (247 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKCMNPPDNIKDTKSDIIFFQRSVPGHDNKMQFESSSYEGYFLACEKERDLFKLILKKEDELGDRSIMFTVQNED

SEQ ID NO: 3 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKCMNPPDNIKDTKSDIIFFQRSVPGHDNKMQFESSSYEGYFLAAEKERDLFKLILKKEDELGDRSIMFTVQNED

SEQ ID NO: 4 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKECNPPDNIKDTKSDIIFFQRSVPGHDNKMQFESSSYEGYFLACEKERDLFKLILKKEDELGDRSIMFTVQNED

SEQ ID NO: 5 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 6 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 7 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 8 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDCKSDIIFFQRSVPGHDNKMQFESSSYEGYFLAAEKERDLFKLILKKEDELGDRSIMFTVQNED

SEQ ID NO: 9 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSCIIFFQRSVPGHDNKMQFESSSYEGYFLACEKERDLFKLILKKEDELGDRSIMFTVQNED

SEQ ID NO: 10 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 11 - protein, 157 aa, synthetic construct
YFGKLKSKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 12 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 13 - protein, 157 aa, synthetic construct
YFGKLKSKLSVIRNLNDQVLFIDQGNRPLFEDMTDSDCRDNAPRTIFIISMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 157 aa, synthetic construct
YFGKLKSKLSVIRNLNDQVLFIDQGNRPLFEDMTDSDCRDNAPRTIFIIAMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, synthetic construct
YFGKLKSKLSVIRNLNDQVLFIDQGNRPLFEDMTDSDCRDNAPRTIFIIHMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 157 aa, synthetic construct
YFGKLKSKLSVIRNLNDQVLFIDQGNRPLFEDMTDSDCRDNAPRTIFIISMYADSQPRGMAVNIAVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 157 aa, synthetic construct
YFGKLKSKLSVIRNLNDQVLFIDQGNRPLFEDMTDSDCRDNAPRTIFIIHMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 18 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENAIISFKEMNPPDNIKDTKSDIIFFQRSVPGHDNKMQFESSSYEGYFLACEKERDLFKLILKKEDELGDRSIMFTVQNED

SEQ ID NO: 20 - protein, 157 aa, synthetic construct; identical to SEQ ID NO: 19

SEQ ID NO: 21 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPAHDNKMQFESSSYEGYFLACEKERDLFKLILKKEDELGDRSIMFTVQNED

SEQ ID NO: 22 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGADNKMQFESSSYEGYFLACEKERDLFKLILKKEDELGDRSIMFTVQNED

SEQ ID NO: 23 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNAMQFESSSYEGYFLACEKERDLFKLILKKEDELGDRSIMFTVQNED

SEQ ID NO: 24 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 157 aa, synthetic construct
YFGKLKSKLSVIRNLNDQVLFIDQGNRPLFEDMTDSDQRDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 26 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 27 - protein, 157 aa, synthetic construct
YFGKLKSKLSVIRNLNDQVLFIDQGNRPLFEDMTDSDQRDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 28 - protein, 157 aa, synthetic construct; identical to SEQ ID NO: 12

SEQ ID NO: 29 - protein, 157 aa, synthetic construct
YFGKLKSKLSIIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 30 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 31 - protein, 157 aa, synthetic construct
YFGKLESKLSVIRNLNDQVLFIDQGNRPLFEDMTDSDARDNAPRTIFIISMYKDSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 32 - protein, 157 aa, synthetic construct
YFGKLESKLSVIRNLNDQVLFIDQGNRPLFEDMTDSDARDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 33 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 34 - protein, 157 aa, synthetic construct
YFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 35 - protein, 157 aa, synthetic construct
GAGKLKSKLSVIRNLNDQVLFIDQGNRPLFEDMTDSDCRDNAPRTIFIISGYAAAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 38 - protein, 157 aa, synthetic construct
YFGKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 39 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 40 - protein, 157 aa, synthetic construct
YFGKLKSKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 42 - protein, 157 aa, synthetic construct
YFGKLKSKLSVIRNLNDQVLFIDQGNRPLFEDMTDSDCRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 43 - protein, 157 aa, synthetic construct
GFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 44 - protein, 157 aa, synthetic construct
YAGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 45 - protein, 157 aa, synthetic construct
YFGKLKSKLSVIRNLNDQVLFIDQGNRPLFEDMTDSDCRDNAPRTIFIISMYAAAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 157 aa, synthetic construct
YFGKLKSKLSVIRNLNDQVLFIDQGNRPLFEDMTDSDCRDNAPRTIFIISGYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 47 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 48 - protein, 157 aa, synthetic construct
YFGKLESKLSVIRNLNDQVLFIDQGNRPLFEDMTDSDSRDNAPRTIFIISMYKDSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 49 - protein, 157 aa, synthetic construct
YFGKLKSKLSVIRNLNDQVLFIDQGNRPLFEDMTDSDSRDNAPRTIFIISMYADAQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 50 - protein, 157 aa, synthetic construct
YFGKLKSKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 52 - protein, 157 aa, synthetic construct
YFGKLKSKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 54 - protein, 157 aa, synthetic construct
YFGKLKSKLSVIRNLNDQVLFIDQGNRPLFEDMTDSDSRDNAPRTIFIISMYADSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDSRDNAPRTIFIISMYADSQPRGMAVAISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 56 - protein, 157 aa, synthetic construct
YFGKLKSKLSVIRNLNDQVLFIDQGNRPLFEDMTDSDSRDNAPRTIFIISMYADSQPRGMAVTISVKCEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 57 - protein, 157 aa, synthetic construct
YFGKLKSKLSVIRNLNDQVLFIDQGNRPLFEDMTDSDSRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 58 - protein, 157 aa, synthetic construct; identical to SEQ ID NO: 54

SEQ ID NO: 59 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 60 - protein, 157 aa, synthetic construct
YFGKLKSKLSVIRNLNDQVLFIDQGNRPLFEDMTDSDQRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 61 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 62 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 63 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 64 - protein, 157 aa, synthetic construct
YFGKLKSKLSVIRNLNDQVLFIDQGNRPLFEDMTDSDARDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 65 - protein, 157 aa, synthetic construct
YFGKLKSKLSVIRNLNDQVLFIDQGNRPLFEDMTDSDQRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 66 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFAEMNPPDNIKDTKSDIIFFQRSVPGHDNKMQFESSSYEGYFLACEKERDLFKLILKKEDELGDRSIMFTVQNED

SEQ ID NO: 67 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSAIIFFQRSVPGHDNKMQFESSSYEGYFLACEKERDLFKLILKKEDELGDRSIMFTVQNED

SEQ ID NO: 68 - protein, 193 aa, synthetic construct
MAAEPVEDNCINFVAMKFIDNTLYFIAEDDENLESDYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 69 - protein, 208 aa, synthetic construct
MAAEPVEDNCINFVAMKFIDNTLYFIAEDDENLESDSGGPGPAGMKGLPGSYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 70 - protein, 196 aa, synthetic construct
MAAEPVEDNCINFVAMKFIDNTLYFIAEDDENLESDPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 71 - protein, 196 aa, synthetic construct
MAAEPVEDNCINFVAMKFIDNTLYFIAEDDENLESDPLGMFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 72 - protein, 199 aa, synthetic construct
MAAEPVEDNCINFVAMKFIDNTLYFIAEDDENLESDPLGLAGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 73 - protein, 284 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNESGGPGPAGMKGLPGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 74 - protein, 276 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEPLGLAGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 75 - protein, 284 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNESGGPGPAGMKGLPGSNIVPVLLGPKLNHVAVELGKNVRLDCSALLNEEDVIYWMFGEEDGSDPNIHEEKEMRIMTPEGKWHASKVLRIENIGESNLNVLYDCTVASTGGTDTKSFILVRKADHHHHHH

SEQ ID NO: 76 - protein, 280 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNESGGPLGLAGGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 77 - protein, 275 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEGGSGGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 78 - protein, 284 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEGGGGSGGGGSGGGGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 79 - protein, 157 aa, synthetic construct
YFGKLESKLSIIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 80 - protein, 157 aa, synthetic construct
YFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 81 - protein, 158 aa, synthetic construct
GYFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 82 - protein, 158 aa, synthetic construct
GYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 83 - protein, 161 aa, synthetic construct
GGGGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 84 - protein, 6 aa, synthetic construct
HHHHHH

SEQ ID NO: 85 - protein, 8 aa, synthetic construct
WSHPQFEK

SEQ ID NO: 86 - protein, 9 aa, synthetic construct
AWAHPQPGG

SEQ ID NO: 87 - protein, 52 aa, synthetic construct
TTNPGVSAWQVNTAYTAGQLVTYNGKTYKCLQPHTSLAGWEPSNVPALWQLQ

SEQ ID NO: 88 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 89 - protein, 36 aa, synthetic construct
MAAEPVEDNCINFVAMKFIDNTLYFIAEDDENLESD

SEQ ID NO: 90 - protein, 36 aa, synthetic construct
MAAEPVEDNSINFVAMKFIDNTLYFIAEDDENLESD

SEQ ID NO: 91 - protein, 36 aa, synthetic construct
MAAEPVEDNAINFVAMKFIDNTLYFIAEDDENLESD

SEQ ID NO: 92 - protein, 36 aa, synthetic construct
MAAEPVEDNCINFVAMKFIDNTLYFIAEDDENLPLG

SEQ ID NO: 93 - protein, 108 aa, synthetic construct
SNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKAD

SEQ ID NO: 94 - protein, 108 aa, synthetic construct
SNIVPVLLGPKLNHVAVELGKNVRLDCSALLNEEDVIYWMFGEEDGSDPNIHEEKEMRIMTPEGKWHASKVLRIENIGESNLNVLYDCTVASTGGTDTKSFILVRKAD

SEQ ID NO: 95 - protein, 541 aa, Homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 96-99 - skipped (4 entries)

SEQ ID NO: 100 - protein, 14 aa, synthetic construct
SGGPGPAGMKGLPG

SEQ ID NO: 101 - protein, 15 aa, synthetic construct
SGGPGPAGMKGLPGS

SEQ ID NO: 102 - protein, 16 aa, synthetic construct
EAGRSANHEPLGLVAT

SEQ ID NO: 103 - protein, 10 aa, synthetic construct
PQASTGRSGG

SEQ ID NO: 104 - protein, 10 aa, synthetic construct
PQGSTGRAAG

SEQ ID NO: 105 - protein, 10 aa, synthetic construct
PPASSGRAGG

SEQ ID NO: 106 - protein, 16 aa, synthetic construct
RSGVPLSLYSGSGGGK

SEQ ID NO: 107 - protein, 14 aa, synthetic construct
RSGMPYDLYHPSGK

SEQ ID NO: 108 - protein, 14 aa, synthetic construct
RGPDSGGFMLTSGK

SEQ ID NO: 109 - protein, 15 aa, synthetic construct
RGSGHEQLTVSGGSK

SEQ ID NO: 110 - protein, 14 aa, synthetic construct
RSGRAAAVKSPSGK

SEQ ID NO: 111 - protein, 20 aa, synthetic construct
RGSGISSGLLSGRSDNHSGK

SEQ ID NO: 112 - protein, 14 aa, synthetic construct
RGDLLAVVAASGGK

SEQ ID NO: 113 - protein, 15 aa, synthetic construct
RGGISSGLLSGRSGK

SEQ ID NO: 114 - protein, 15 aa, synthetic construct
SGGGKKLADEPEGGS

SEQ ID NO: 115 - protein, 13 aa, synthetic construct
GGGKFLADEPEGG

SEQ ID NO: 116 - protein, 8 aa, synthetic construct
ARLQSAAP

SEQ ID NO: 117 - protein, 14 aa, synthetic construct
ARLQSAAPAGLKGA

SEQ ID NO: 118 - protein, 17 aa, synthetic construct
GSGGPGPAGMHGLPGGS

SEQ ID NO: 119 - protein, 16 aa, synthetic construct
GGGSHTGRSAYFGGGS

SEQ ID NO: 120 - protein, 15 aa, synthetic construct
SGGPGPAGLKGAPGS

SEQ ID NO: 121 - protein, 8 aa, synthetic construct
VPLSLYSG

SEQ ID NO: 122 - protein, 11 aa, synthetic construct
SGLLSGRSDNH

SEQ ID NO: 123 - protein, 11 aa, synthetic construct
SGRSDNIGGGS

SEQ ID NO: 124 - protein, 15 aa, synthetic construct
LQESLRSKESGRSDI

SEQ ID NO: 125 - protein, 13 aa, synthetic construct
ISSGLLSGRSDNH

SEQ ID NO: 126 - protein, 8 aa, synthetic construct
LSGRSDDH

SEQ ID NO: 127 - protein, 13 aa, synthetic construct
ISSGLLSGRSDQH

SEQ ID NO: 128 - protein, 13 aa, synthetic construct
ISSGLLSGRSDNI

SEQ ID NO: 129 - protein, 6 aa, synthetic construct
PLGLAG

SEQ ID NO: 130 - protein, 8 aa, synthetic construct
GPAGMKGL

SEQ ID NO: 131 - protein, 8 aa, synthetic construct
LSGRSDQH

SEQ ID NO: 132 - protein, 8 aa, synthetic construct
LSGRSDNI

SEQ ID NO: 133 - protein, 13 aa, synthetic construct
ISSGLLSGRSDNH

SEQ ID NO: 134 - protein, 7 aa, synthetic construct
GPLGVRG

SEQ ID NO: 135 - protein, 7 aa, synthetic construct
GPLGLAR

SEQ ID NO: 136 - protein, 8 aa, synthetic construct
GPAALVGA

SEQ ID NO: 137 - protein, 8 aa, synthetic construct
GPAALIGG

SEQ ID NO: 138 - protein, 8 aa, synthetic construct
GPLNLVGR

SEQ ID NO: 139 - protein, 8 aa, synthetic construct
GPAGLVAD

SEQ ID NO: 140 - protein, 8 aa, synthetic construct
GPANLVAP

SEQ ID NO: 141 - protein, 8 aa, synthetic construct
VPLSLYSG

SEQ ID NO: 142 - protein, 10 aa, synthetic construct
SGESPAYYTA

SEQ ID NO: 143 - protein, 7 aa, synthetic construct
GGPRGLG

SEQ ID NO: 144 - protein, 6 aa, synthetic construct
HSSKLQ

SEQ ID NO: 145 - protein, 7 aa, synthetic construct
HSSKLQL

SEQ ID NO: 146 - protein, 8 aa, synthetic construct
KRALGLPG

SEQ ID NO: 147 - protein, 5 aa, synthetic construct
LEATA

SEQ ID NO: 148 - protein, 10 aa, synthetic construct
GGAANLVRGG

SEQ ID NO: 149 - protein, 10 aa, synthetic construct
SGRIGFLRTA

SEQ ID NO: 150 - protein, 5 aa, synthetic construct
PLGLA

SEQ ID NO: 151 - protein, 8 aa, synthetic construct
ESPAYYTA

SEQ ID NO: 152 - protein, 6 aa, synthetic construct
RLQLKL

SEQ ID NO: 153 - protein, 7 aa, synthetic construct
RLQLKAC

SEQ ID NO: 154 - protein, 5 aa, synthetic construct
SGRSA

SEQ ID NO: 155 - skipped

SEQ ID NO: 156 - protein, 5 aa, synthetic construct
GGGRR

SEQ ID NO: 157 - protein, 4 aa, synthetic construct
GFLG

SEQ ID NO: 158 - protein, 9 aa, synthetic construct
HSSKLQEDA

SEQ ID NO: 159 - protein, 10 aa, synthetic construct
LVLASSSFGY

SEQ ID NO: 160 - protein, 10 aa, synthetic construct
GVSQNYPIVG

SEQ ID NO: 161 - protein, 10 aa, synthetic construct
GVVQASCRLA

SEQ ID NO: 162 - protein, 6 aa, synthetic construct
DPRSFL

SEQ ID NO: 163 - protein, 6 aa, synthetic construct
PPRSFL

SEQ ID NO: 164 - protein, 4 aa, synthetic construct
DEVD

SEQ ID NO: 165 - protein, 5 aa, synthetic construct
DEVDP

SEQ ID NO: 166 - protein, 8 aa, synthetic construct
KGSGDVEG

SEQ ID NO: 167 - protein, 6 aa, synthetic construct
GWEHDG

SEQ ID NO: 168 - protein, 7 aa, synthetic construct
EDDDDKA

SEQ ID NO: 169 - protein, 9 aa, synthetic construct
KQEQNPGST

SEQ ID NO: 170 - protein, 6 aa, synthetic construct
GKAFRR

SEQ ID NO: 171 - protein, 4 aa, synthetic construct
DAFK

SEQ ID NO: 172 - protein, 4 aa, synthetic construct
DVLK

SEQ ID NO: 173 - protein, 4 aa, synthetic construct
DAFK

SEQ ID NO: 174 - protein, 7 aa, synthetic construct
ALLLALL

SEQ ID NO: 175 - protein, 10 aa, synthetic construct
SGAKPRALTA

SEQ ID NO: 176 - protein, 10 aa, synthetic construct
SGLRLAAITA

SEQ ID NO: 177 - protein, 10 aa, synthetic construct
SGESLAYYTA

SEQ ID NO: 178 - protein, 10 aa, synthetic construct
SGESPAYYTA

SEQ ID NO: 179 - protein, 10 aa, synthetic construct
SGESLRYYTA

SEQ ID NO: 180 - protein, 10 aa, synthetic construct
SGRSLSRLTA

SEQ ID NO: 181 - protein, 10 aa, synthetic construct
SGRSLRRLTA

SEQ ID NO: 182 - protein, 10 aa, synthetic construct
SGAVSWLLTA

SEQ ID NO: 183 - protein, 10 aa, synthetic construct
SGAPSWLLTA

SEQ ID NO: 184 - protein, 10 aa, synthetic construct
SGAVRWLLTA

SEQ ID NO: 185 - protein, 10 aa, synthetic construct
SGANISDLTA

SEQ ID NO: 186 - protein, 10 aa, synthetic construct
SGNRYSSLTA

SEQ ID NO: 187 - protein, 10 aa, synthetic construct
SGHMHKALTA

SEQ ID NO: 188-189 - skipped (2 entries)

SEQ ID NO: 190 - protein, 12 aa, synthetic construct
GEEGEEPLGLAG

SEQ ID NO: 191 - protein, 7 aa, synthetic construct
GPLGLAG

SEQ ID NO: 192 - protein, 16 aa, synthetic construct
EAGRSANHTPAGLTGP

SEQ ID NO: 193 - protein, 17 aa, synthetic construct
GEAGRSANHTPAGLTGP

SEQ ID NO: 194-200 - skipped (7 entries)

SEQ ID NO: 201 - protein, 8 aa, synthetic construct
KRALGLPG

SEQ ID NO: 202 - skipped

SEQ ID NO: 203 - protein, 5 aa, synthetic construct
LEATA

SEQ ID NO: 204 - protein, 10 aa, synthetic construct
GGAANLVRGG

SEQ ID NO: 205 - protein, 10 aa, synthetic construct
SGRIGFLRTA

SEQ ID NO: 206 - protein, 6 aa, synthetic construct
PLGLAG

SEQ ID NO: 207 - protein, 6 aa, synthetic construct
  VARIANT 6: note = Any amino acid
PLGLAX

SEQ ID NO: 208 - protein, 8 aa, synthetic construct
ESPAYYTA

SEQ ID NO: 209 - protein, 6 aa, synthetic construct
RLQLKL

SEQ ID NO: 210 - protein, 7 aa, synthetic construct
RLQLKAC

SEQ ID NO: 211 - protein, 5 aa, synthetic construct
SGRSA

SEQ ID NO: 212 - protein, 4 aa, synthetic construct
DAFK

SEQ ID NO: 213 - protein, 5 aa, synthetic construct
GGGRR

SEQ ID NO: 214 - protein, 4 aa, synthetic construct
GFLG

SEQ ID NO: 215 - protein, 4 aa, synthetic construct
ALAL

SEQ ID NO: 216-217 - skipped (2 entries)

SEQ ID NO: 218 - protein, 5 aa, synthetic construct
GLLYA

SEQ ID NO: 219 - protein, 8 aa, synthetic construct
GGPRGLPG

SEQ ID NO: 220 - protein, 6 aa, synthetic construct
HSSKLQ

SEQ ID NO: 221 - protein, 7 aa, synthetic construct
HSSKLQL

SEQ ID NO: 222 - protein, 9 aa, synthetic construct
HSSKLQEDA

SEQ ID NO: 223 - protein, 6 aa, synthetic construct
DPRSFL

SEQ ID NO: 224 - protein, 6 aa, synthetic construct
PPRSFL

SEQ ID NO: 225 - protein, 4 aa, synthetic construct
DEVD

SEQ ID NO: 226 - protein, 5 aa, synthetic construct
DEVDP

SEQ ID NO: 227 - protein, 8 aa, synthetic construct
KGSGDVEG

SEQ ID NO: 228 - protein, 6 aa, synthetic construct
GWEHDG

SEQ ID NO: 229 - protein, 9 aa, synthetic construct
KQEQNPGST

SEQ ID NO: 230 - protein, 6 aa, synthetic construct
GKAFRR

SEQ ID NO: 231 - protein, 4 aa, synthetic construct
DAFK

SEQ ID NO: 232 - protein, 4 aa, synthetic construct
DVLK

SEQ ID NO: 233 - protein, 4 aa, synthetic construct
DAFK

SEQ ID NO: 234 - protein, 7 aa, synthetic construct
ALLLALL

SEQ ID NO: 235 - protein, 20 aa, synthetic construct
  VARIANT 1..20: note = This sequence may encompass 1-10 GS repeating units
GSGSGSGSGSGSGSGSGSGS

SEQ ID NO: 236 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 237 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 238 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 239 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 240 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 241 - protein, 15 aa, synthetic construct
GGGGSGGGGSGGGGS

SEQ ID NO: 242 - protein, 20 aa, synthetic construct
GGGGSGGGGSGGGGSGGGGS

SEQ ID NO: 243 - protein, 14 aa, synthetic construct
GGGGSGGGGSGGGG

SEQ ID NO: 244 - protein, 5 aa, synthetic construct
GGSGG

SEQ ID NO: 245 - protein, 5 aa, synthetic construct
SDGTK

SEQ ID NO: 246 - protein, 6 aa, synthetic construct
MSDGTK

SEQ ID NO: 247 - protein, 7 aa, synthetic construct
AMSDGTK

(19) United States
(12) Patent Application Publication
(10) Pub. No.: US 2024/0116997 A1
(43) Pub. Date: Apr. 11, 2024

(54) ACTIVATABLE IL-18 POLYPEPTIDES

(71) Applicant: Bright Peak Therapeutics AG, Basel (CH)

(72) Inventors: Vijaya Raghavan PATTABIRAMAN, Volketswil (CH); Bertolt KREFT, Kleinmachow (DE); Arnaud GOEPFERT, Sierentz (FR); Tiziano ONGARO, Basel (CH)

(21) Appl. No.: 18/113,399

(22) Filed: Feb. 23, 2023

Related U.S. Application Data
(60) Provisional application No. 63/313,210, filed on Feb. 23, 2022.

(51) Int. Cl. C07K 14/54

(52) U.S. Cl. CPC C07K 14/54; C07K 2319/50

(57) ABSTRACT

The present disclosure relates to activatable IL-18 polypeptides, compositions comprising activatable IL-18 polypeptides, methods of making the same, and methods of using the activatable IL-18 polypeptides for treatment of diseases. In one aspect, the disclosure relates to the treatment of cancer using the activatable IL-18 polypeptides.

### CROSS REFERENCE

This application claims the benefit of U.S. Provisional Application No. 63/313,210 filed Feb. 23, 2022, which application is incorporated herein by reference in its entirety.

### SEQUENCE LISTING

The instant application contains a Sequence Listing which has been submitted electronically in XML format and is hereby incorporated by reference in its entirety. Said XML copy, created on Apr. 5, 2023, is named 94917-0076_719201US_SL.xml and is 220,945 bytes in size.

### BACKGROUND

Immunotherapies utilize the immune system of a subject to aid in the treatment of ailments. Immunotherapies can be designed to activate or suppress the immune system depending on the nature of the disease being treated. The goal of immunotherapies for the treatment of cancer is to stimulate the immune system so that it recognizes and destroys tumors or other cancerous tissue. One method of activating the immune system to attack cancer cells in the body of a subject is cytokine therapy. Cytokines are proteins produced in the body that are important in cell signaling and in modulating the immune system. Some cytokine therapy utilizes these properties of cytokines to enhance the immune system of a subject to kill cancer cells.

### BRIEF SUMMARY

Provided herein are activatable IL-18 (Act-IL-18) polypeptides which are activated in response to certain conditions or stimuli found in a target area of a subject after administration. In some cases, the Act-IL-18s are administered in an inactivated form and later release an active form of an IL-18 polypeptide upon contact with the condition or stimulus. In some cases, this allows the IL-18 polypeptide to modulate an immune response preferentially in a target area of the subject, such as a cancer or tumor microenvironment. In some instances, the Act-IL-18s exhibit fewer side effects associated with systemic administration and distribution of the corresponding active IL-18 polypeptide. In some embodiments, an Act-IL-18 of the instant disclosure comprises an artificial terminal moiety, such as a peptide, attached to a terminus of an IL-18 polypeptide (e.g., the N-terminus or the C-terminus) which serves to inactivate the IL-18 polypeptide. In some embodiments, the artificial terminal moiety is cleaved under one or more conditions associated with a desired target are (e.g., a tumor microenvironment), thus releasing an active IL-18 polypeptide.

FIG. 1A depicts an exemplary mechanism of action of an activatable IL-18 polypeptide as provided herein, wherein the IL-18 polypeptide comprises an artificial terminal moiety which renders the Act-IL-18 inactive and upon cleavage of the artificial terminal moiety, the active form of the IL-18 polypeptide results. FIG. 1B illustrates a similar concept with artificial terminal moiety attached to the N-terminus of the IL-18 polypeptide. The artificial terminal moiety depicted comprises a linking peptide B attaching the protease cleavage site having a specific cleavage site to the N-terminus of the IL-18 polypeptide. The artificial terminal moiety further comprises a linking peptide A linking the protease cleavage site to a blocking moiety (labelled “mask” in the figure). Upon cleavage of the specific cleavage site, the mask and linking peptide A are released from the IL-18 polypeptide, but linking peptide B remains. This results in an active form of the IL-18 polypeptide being formed. FIG. 1C is analogous to FIG. 1B, but the artificial terminal moiety is linked to the C-terminus of the IL-18 polypeptide.

In some embodiments, an Act-IL-18 comprises an artificial terminal moiety which comprises a blocking group linked to the IL-18 polypeptide. In some embodiments, the blocking moiety is positions such that cleavage of the artificial terminal moiety releases the blocking moiety from the IL-18 polypeptide, thereby allowing the IL-18 polypeptide to interact with the receptor (or interact with the receptor to a higher degree). In some embodiments, the blocking moiety comprises the IL-18 propeptide (e.g., the N-terminal portion of immature IL-18 which is endogenously cleaved by caspases to produce mature IL-18). In some embodiments, the IL-18 propeptide is linked to the IL-18 polypeptide specific cleavage site which is cleaved by a protease other than a caspase (e.g., a protease which is upregulated in a tumor or tumor microenvironment). In some embodiments, the IL-18 propeptide is linked to the N-terminus of the IL-18 polypeptide. In some embodiments, the blocking moiety comprises a domain of an IL-18 receptor subunit, such as the D3 domain of the IL-18 receptor alpha subunit (see, e.g., Tsutsumi et al., Nature Communications 5:5340 DOI: 10.1038/ncomms6340, published 15 Dec. 2014, for a description of IL-18 receptor domain architecture).

In one aspect, provided herein, is an activatable interleukin-18 (Act-IL-18) polypeptide comprising: an artificial terminal moiety attached to an interleukin-18 (IL-18) polypeptide, wherein the artificial terminal moiety comprises a specific cleavage site, and wherein cleavage at the specific cleavage site converts the Act-IL-18 into an active form of the IL-18 polypeptide. In some embodiments, when the artificial terminal moiety is intact, the Act-IL-18 is inactive.

In some embodiments, the specific cleavage site is preferentially cleaved at or near a target tissue of a subject. In some embodiments, the specific cleavage site is preferentially cleaved in or near a tumor microenvironment.

In some embodiments, the specific cleavage site is specifically cleaved by a protease. In some embodiments, the protease is found at higher concentrations and/or demonstrates higher proteolytic activity within the tumor microenvironment relative to non-tumor tissue. In some embodiments, the protease is selected from: kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, and a tumor cell surface protease. In some embodiments, the artificial terminal moiety comprises a peptide having a sequence at least 80%, at least 85%, at least 90%, at least 95%, at least 96%, at least 97%, at least 98%, at least 99%, or 100% identical to a peptide sequence set forth in Table 2A. In some embodiments, the artificial terminal moiety comprises a peptide having a sequence at least 80%, at least 85%, at least 90%, at least 95%, at least 96%, at least 97%, at least 98%, at least 99%, or 100% identical to a peptide sequence set forth in Table 2B.

In some embodiments, the specific cleavage site is a redox sensitive cleavage site. In some embodiments, the redox sensitive cleavage site is preferentially cleaved in a reducing environment. In some embodiments, the specific cleavage site is a pH sensitive cleavage site. In some embodiments, the pH sensitive cleavage site is preferentially cleaved at a pH below 7.3, below 7.2, below 7.1, or below 7.0.

In some embodiments, the cleavage removes the entire artificial terminal moiety from the IL-18 polypeptide. In some embodiments, the cleavage results in a portion of the artificial moiety remaining attached to the IL-18 polypeptide.

In some embodiments, the artificial terminal moiety is a peptide. In some embodiments, cleavage of the artificial terminal moiety at the specific cleavage site leave no amino acid residues of the peptide attached to the IL-18 polypeptide. In some embodiments, cleavage of the artificial terminal moiety at the specific cleavage site leaves at least 1 amino acid residue of the peptide attached to the IL-18 polypeptide. In some embodiments, cleavage of the artificial terminal moiety at the specific cleavage site leaves 1, 2, 3, 4, 5, 6, 7, 8, 9, or 10 amino acid residues of the peptide attached to the IL-18 polypeptide. In some embodiments, the peptide comprises multiple specific cleavage site. In some embodiments, the peptide comprises 2, 3, or 4 specific cleavage sites. In some embodiments, the peptide is between 2 and 35 amino acid residues in length. In some embodiments, the peptide is 8, 9, or 10 amino acid residues in length.

In some embodiments, the artificial terminal moiety is attached to the N-terminus or the C-terminus of the IL-18 polypeptide.

In some embodiments, the artificial terminal moiety is attached to the N-terminus of the IL-18 polypeptide. In some embodiments, the artificial terminal moiety comprises a blocking moiety. In some embodiments, the blocking moiety is positioned such that cleavage at the specific cleavage site releases the blocking moiety from the Act-IL-18 polypeptide. In some embodiments, the blocking moiety comprises an IL-18 propeptide or a portion thereof, or a variant thereof. In some embodiments, the IL-18 propeptide is a human IL-18 propeptide or a variant thereof. In some embodiments, the IL-18 propeptide comprises an amino acid sequence having at least 80%, 85%, 90%, 95%, 96%, 97%, 98%, 99%, or 100% sequence identity to the sequence set forth in SEQ ID NO: 89. In some embodiments, the artificial terminal moiety comprises a linking peptide between the specific cleavage site and the blocking moiety.

In some embodiments, the artificial terminal moiety is attached to the C-terminus of the IL-18 polypeptide. In some embodiments, wherein the blocking moiety is positioned such that cleavage at the specific cleavage site releases the blocking moiety from the Act-IL-18 polypeptide. In some embodiments, the blocking moiety comprises a domain of an IL-18 receptor subunit or a portion thereof, or a derivative thereof. In some embodiments the domain of the IL-18 receptor subunit comprises the D3 domain of the IL-18 receptor alpha subunit, or a variant thereof. In some embodiments, the blocking moiety comprises an amino acid sequence having at least 80%, 85%, 90%, 95%, 96%, 97%, 98%, 99%, or 100% sequence identity to the sequence set forth in SEQ ID NO: 93. In some embodiments, the artificial terminal moiety comprises a linking peptide between the specific cleavage site and the blocking moiety. In some embodiments, the Act-IL-18 polypeptide comprises a linking peptide between the IL-18 polypeptide and the specific cleavage site.

In some embodiments, the active form of the IL-18 polypeptide displays reduced binding to IL-18 binding protein (IL-18BP) compared to WT IL-18. In some embodiments, the active form of the IL-18 polypeptide displays enhanced binding to IL-18R or ability to activate IL-18R. In some embodiments, the active form of the IL-18 polypeptide displays a binding to IL-18R or ability to activate IL-18R which is reduced by at most 100-fold relative to WT IL-18.

In some embodiments, the IL-18 polypeptide comprises one or more modifications to the sequence set forth in SEQ ID NO: 1. In some embodiments, the IL-18 polypeptide comprises an amino acid sequence having at least about 80%, at least about 85%, at least about 90%, at least about 95%, at least about 96%, at least about 97%, at least about 98%, or at least about 99% sequence identity to the sequence set forth in SEQ ID NO: 1. In some embodiments, the IL-18 polypeptide comprises at least one substitution at residue Y1, F2, E6, C38, K53, D54, S55, T63, C76, E85, M86, T95, D98, or C127, or any combination thereof. In some embodiments, the IL-18 polypeptide comprises a Y01G, F02A, E06K, V11I, C38S, C38A, K53A, D54A, S55A, T63A, C76S, C76A, E85C, M86C, T95C, D98C, C127S, or C127A amino acid substitution, or any combination thereof. In some embodiments, the IL-18 polypeptide comprises E06K and K53A amino acid substitutions. In some embodiments, the IL-18 polypeptide comprises a T63A amino acid substitution. In some embodiments, the IL-18 polypeptide comprises a VIII amino acid substitution. In some embodiments, the IL-18 polypeptide comprises substitutions at 1, 2, 3, or 4 residues selected from C38, C76, C98, and C127. In some embodiments, the IL-18 polypeptide comprises an amino acid sequence set forth in any one of SEQ ID Nos: 2-67. In some embodiments, the IL-18 polypeptide comprises the amino acid sequence set forth in SEQ ID NO: 30. In some embodiments, the IL-18 polypeptide comprises an amino acid sequence as set forth in any one of SEQ ID NOs: 79-83.

In some embodiments, the Act IL-18 polypeptide exhibits a half-maximal effective concentration (EC₅₀) for IL-18 receptor signaling activity which is at least 1,000-fold higher, 2,000-fold higher, 5,000-fold higher, 10,000-fold higher, 15,000-fold-higher, or 20,000-fold higher than the activated form of the IL-18 polypeptide. In some embodiments, the Act IL-18 polypeptide exhibits a half-maximal effective concentration (EC₅₀) for IL-18 receptor signaling activity which is from about 10-fold higher to about 100-fold higher than the activated form of the IL-18 polypeptide. In some embodiments, the activated form of the IL-18 polypeptide exhibits a half-maximal effective concentration (EC₅₀) for IL-18 receptor signaling activity which is within about 10-fold of the IL-18 polypeptide. In some embodiments, the Act-IL-18 polypeptide exhibits a half-maximal effective concentration (EC₅₀) for IL-18 receptor signaling activity which is at least 10-fold greater than WT IL-18.

In some embodiments, the IL-18 polypeptide is synthetic.

In some embodiments, a polymer is attached to a residue of the IL-18 polypeptide.

In another aspect is method of treating cancer in a subject in need thereof comprising administering to the subject a pharmaceutically effective amount of an Act-IL-18 polypeptide or a pharmaceutical composition provided herein. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer. In some embodiments, the solid cancer is a carcinoma or a sarcoma. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia. In some embodiments, the method comprises reconstituting a lyophilized form of the Act-IL-18 polypeptide or the pharmaceutical composition.

Another aspect provides a method of making an Act-IL-18 polypeptide provided herein comprising synthesizing two or more fragments of the Act-IL-18 polypeptide, ligating the fragments, and folding the ligated fragments.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1A shows an illustration of an exemplary mechanism of activation of an Act-IL-18 as provided herein.

FIG. 1B shows an illustration of an exemplary mechanism of activation of an Act-IL-18 polypeptide having a blocking moiety (“mask” in the figure) linked to the N-terminus of the IL-18 polypeptide.

FIG. 1C shows an illustration of an exemplary mechanism of activation of an Act-IL-18 polypeptide having a blocking moiety (“mask” in the figure) linked to the C-terminus of the IL-18 polypeptide.

FIG. 2 illustrates the mechanism of action of IL-18 on IFNγ and IL-18BP production, and IL-18 inhibitory activity by IL-18BP.

FIG. 3 illustrates the coupling of a dibenzocyclooctyne (DBCO) polyethylene glycol (PEG) with a IL-18 polypeptide comprising an azide.

FIG. 4 illustrates the binding of an IL-18 polypeptide comprising a polymer with IL-18Rα.

FIG. 5A shows the IFNγ induction ability of an IL-18 polypeptide of the disclosure compared to a wild type IL-18 polypeptide.

FIG. 5B shows IL-18BP inhibition of an IL-18 polypeptide of the disclosure compared to a wild type IL-18 polypeptide.

FIG. 6 shows a schematic representation of coupling of a bifunctional probe to an IL-18 polypeptide provided herein.

FIG. 7 shows a schematic representation of coupling of a poly(ethylene glycol) moiety to an IL-18 polypeptide activated with a bifunctional probe.

FIG. 8 shows a conditionally activatable IL-18 with terminal protease recognition sequence linked to a blocking moiety and an orthogonal conjugation handle.

FIG. 9 shows a conditionally activatable IL-18 polypeptide with an N-terminal protease

FIG. 10A shows a conditionally activatable IL-18 with a C-terminal protease recognition sequence and blocking moiety and an orthogonal conjugation handle.

FIG. 10B shows a conditionally activatable IL-18 with a C-terminal protease recognition sequence and an orthogonal conjugation handle.

FIG. 11 shows an illustrative example for a conditionally activatable, conjugatable IL-18 polypeptide with three N-terminal residues substituted with protease recognition sequence and blocking moiety. Upon proteolytic processing in a tumor microenvironment (TME), a functional IL-18 mutein is revealed.

FIG. 12 shows an illustrative example for a conditionally activatable, conjugatable IL-18 polypeptide with three C-terminal residues substituted with protease recognition sequence and blocking moiety. Upon proteolytic processing in TME, a functional IL-18 mutein is revealed.

FIG. 13 shows an illustrative example for a conditionally activatable IL-18 polypeptide with three N-terminal residues substituted with protease recognition sequence and conjugation handle. Upon proteolytic processing in TME, free functional IL-18 mutein is revealed.

FIG. 14A shows SDS-PAGE gels showing activatable IL-18 polypeptides provided herein both before and after MMP treatment.

FIG. 14B shows SDS-PAGE gels showing activatable IL-18 polypeptides provided herein both before and after MMP treatment.

FIG. 14C shows SDS-PAGE gels showing activatable IL-18 polypeptides provided herein after MMP treatment and resin purification. FT indicates flow through and E indicates eluate.

FIG. 15A shows dose response curves for IL-18 receptor activation for activatable IL-18 polypeptides provided herein.

FIG. 15B shows dose response curves for IL-18 receptor activation for activatable IL-18 polypeptides provided herein.

FIG. 16A shows sequences and peptide maps of activatable IL-18 polypeptides provided herein.

FIG. 16B shows sequences and peptide maps of activatable IL-18 polypeptides provided herein.

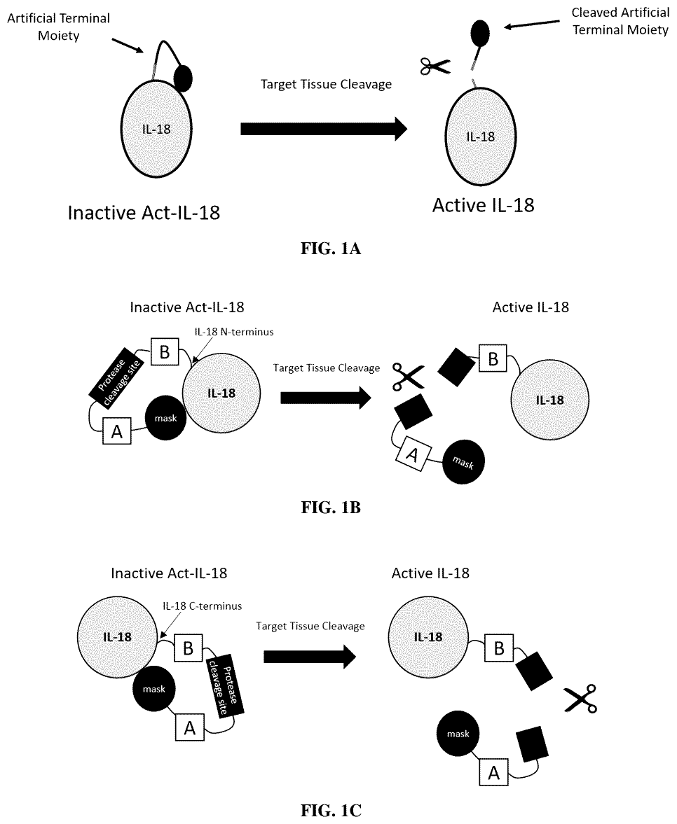

### DETAILED DESCRIPTION

Immune responses to tumors are primarily the function of T helper type 1 (Th1) lymphocytes. Th1 responses include the secretion of cytokines IL-2, IL-12, IL-18, IFNγ, and the generation of specific cytotoxic T lymphocytes that recognize specific tumor antigens. The Th1 response is a vital arm of host defense against many microorganisms. However, the Th1 response is also associated with autoimmune diseases and organ transplant rejection.

Interleukin 18 (IL-18) is a pro-inflammatory cytokine that elicits biological activities that initiate or promote host defense and inflammation following infection or injury. IL-18 has been implicated in autoimmune diseases, myocardial function, emphysema, metabolic syndromes, psoriasis, inflammatory bowel disease, hemophagocytic syndromes, macrophage activation syndrome, sepsis, and acute kidney injury. In some models of disease, IL-18 plays a protective role.

IL-18 also plays a major role in the production of IFNγ from T-cells and natural killer cells. IFNγ is a Th1 cytokine mainly produced by T cells, NK cells, and macrophages and is critical for innate and adaptive immunity against viral, some bacterial, and protozoal infections. IFNγ is also an important activator of macrophages and inducer of Class II major histocompatibility complex (MHC) molecule expression.

IL-18 forms a signaling complex by binding to the IL-18 alpha chain (IL-18Rα), which is the ligand binding chain for mature IL-18. However, the binding affinity of IL-18 to IL-18Rα is low. In cells that express the co-receptor, IL-18 receptor beta chain (IL-18Rβ), a high affinity heterodimer complex is formed, which then activates cell signaling.

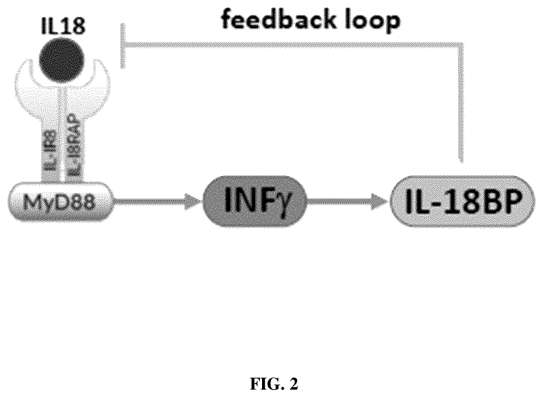

The activity of IL-18 is counteracted by the presence of a high affinity, naturally occurring IL-18 binding protein (IL-18BP). IL-18BP binds IL-18 and neutralizes the biological activity of IL-18. Cell surface IL-18Rα competes with IL-18BP for IL-18 binding. Increased disease severity can be associated with an imbalance of IL-18 to IL-18BP such that levels of free IL-18 are elevated in the circulation. FIG. 2 illustrates the mechanism of action of IL-18, IFNγ production, IL-18BP production, and inhibition of IL-18 activity by IL-18BP. IL-18 induces IFNγ production, which in turn induces IL-18BP production. IL-18BP then competes with IL-18Rα to inhibit IL-18 activity. This feedback loop of IL-18BP production after stimulation of IFNγ production has limited the effectiveness of IL-18 as a treatment modality in previous efforts.

In order to combat this inactivation, variants of IL-18 with reduced binding to IL-18 BP are currently being investigated. Non-limiting examples of such variants include those with amino acid substitutions which inhibit binding with IL-18BP, such as those found in PCT Publication No. WO2019051015A1 and WO2002101049A2, each of which is hereby incorporated by reference as if set forth herein in its entirety. However, high levels of IL-18 in serum has the potential to produce deleterious off-target effect through the indiscriminant activation of the IL-18 receptor in non-target tissues and cells. These off-target effects include lymphopenia, hyperglycemia, anemia, neutropenia, hypoalbuminemia, liver damage, liver enzyme elevation, lymphopenia, increased activation of lymphocytes along with increased serum concentrations of creatinine, IFN-γ, and granulocyte macrophage colony-stimulating factor. Importantly, high levels of circulating free IL-18 that is not bound by neutralizing IL-18BP have been implicated in the development of potentially life-threatening systemic autoinflammatory/autoimmune diseases such as adult-onset Still's disease (AOSD) and systemic juvenile idiopathic arthritis (sJIA) but also in their most severe complication, macrophage activation syndrome (MAS).

In order to prevent immune-related adverse events or minimize these off-target and systemic effects, provided herein are activatable forms of interleukin-18 (Act-IL-18). In some instances, the Act-IL-18 is activated by a condition associated with a target tissue, such as a cancer or tumor microenvironment. In some cases, the Act-IL-18 is preferentially activated within or near the target tissue, thus leading to an elevated local concentration of an active IL-18 relative to non-target tissue. This in turn minimizes the off target effects of the IL-18 but allows the IL-18 to still modulate the local immune response in the target tissue.

In some embodiments, it has been observed herein that small moieties attached to a terminus of an IL-18 polypeptide (e.g., short peptide sequences or small linkers attached to the N- or C-terminus of an IL-18 polypeptide, in particular the N-terminus) can functionally inhibit the activity of the IL-18 polypeptide. While it has been previously observed that endogenous IL-18 is initially expressed with an additional 36 amino acid segment at the N-terminus which is cleaved by caspases, it was previously unknown if other moieties (e.g., shorter peptide sequences) affixed to the terminus of mature IL-18 could inhibit IL-18 activity. Indeed, others have observed that IL-18 fusion proteins which include the 36 amino acid precursor peptide segment retain IL-18 receptor signaling activity (see, e.g., PCT Pub. No. WO2005014642A2, which is hereby incorporated by reference as if set forth herein in its entirety).

In some embodiments, Act-IL-18 polypeptides provided herein utilize blocking moieties attached to the IL-18 polypeptide through cleavable groups in order to inhibit and/or reduce activity of the IL-18 polypeptide. Such blocking moieties can include the IL-18 propeptide attached through a new cleavable group (e.g., not the endogenous cleavable group of full length, immature IL-18) or the D3 domain of the IL-18 receptor alpha subunit. In such instances, cleavage of the specific cleavage group of the Act-IL-18 polypeptide releases blocking moiety and thereby results in an activated form the IL-18 polypeptide.

In one aspect, provided herein, is an Act-IL-18 comprising an artificial terminal moiety which deactivates the IL-18 polypeptide at off-target locations but is cleaved to yield an active IL-18 polypeptide. In some embodiments, the artificial terminal moiety conditionally interferes with binding between the IL-18 receptor (IL-18Rαβ) and the Act-IL-18. In some embodiments, transformation of the artificial terminal moiety by conditions in or near the target tissue (e.g., disease tissue such as a tumor microenvironment) abate the receptor binding interference provided by the artificial terminal moiety. In some embodiments, the artificial terminal moiety interferes with binding between the IL-18 polypeptide and the IL-18 receptor in non-target tissue. In some embodiments, transformation of the artificial terminal moiety (e.g., cleavage of the artificial terminal moiety or other alteration of the artificial terminal moiety, such as oxidation/reduction or conformational change) leads to increased binding affinity and/or activation of IL-18 receptor with the transformed Act-IL-18 relative to Act-IL-18 that has not been transformed. In some embodiments, target tissue specific transformation and activation of the Act-IL-18 provides an active IL-18 for the treatment of tumors with minimal active IL-18 in non-target tissues and cells. In some embodiments, the active form of the IL-18 polypeptide exhibits reduced ability to bind and/or be neutralized by IL-18BP.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment. The section headings used herein are for organizational purposes only and are not to be construed as limiting the subject matter described.

### I. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

As used herein, an “alpha-keto amino acid” or the phrase “alpha-keto” before the name of an amino acid refers to an amino acid or amino acid derivative having a ketone functional group positioned between the carbon bearing the amino group and the carboxylic acid of an amino acid. Alpha-keto amino acids of the instant disclosure have a structure as set forth in the following formula:

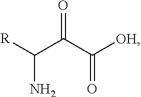

wherein R is the side chain of any natural or unnatural amino acid. The R functionality can be in either the L or D orientation in accordance with standard amino acid nomenclature. In preferred embodiments, alpha-keto amino acids are in the L orientation. When the phrase “alpha-keto” is used before the name of a traditional natural amino acid (e.g., alpha-keto leucine, alpha-keto phenylalanine, etc.) or a common unnatural amino acid (e.g., alpha-keto norleucine, alpha-keto O-methyl-homoserine, etc.), it is intended that the alpha-keto amino acid referred to matches the above formula with the side chain of the referred to amino acid. When an alpha-keto amino acid residue is set forth in a peptide or polypeptide sequence herein, it is intended that a protected version of the relevant alpha-keto amino acid is also encompassed (e.g., for a sequence terminating in a C-terminal alpha-keto amino acid, the terminal carboxylic acid group may be appropriately capped with a protecting group such as a tert-butyl group, or the ketone group with an acetal protecting group). Other protecting groups encompassed are well known in the art.

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is a “corresponding” position through an analysis of the alignment of the two sequences being compared.

Unless otherwise specified, is contemplated that “protected” versions of amino acids (e.g., those containing a chemical protecting group affixed to a functionality of the amino acid, particularly a side chain of the amino acid but also at another point of the amino acid) qualify as the same amino acid as the “unprotected” version for sequence identity purposes, particularly for chemically synthesized polypeptides. It is also contemplated that such protected versions are also encompassed by the SEQ ID NOs provided herein. Non-limiting examples of protecting groups which may be encompassed include fluorenylmethyloxycarbonyl (Fmoc), triphenylmethyl (trityl or trt), tert-Butyloxycarbonyl (Boc), 2,2,4,6,7-pentamethyldihydrobenzofuran-5-sulfonyl (Pbf), acetamidomethyl (Acm), tert-butyl (tBu or OtBu), 2,2-dimethyl-1-(4-methoxyphenyl)propane-1,3-diol ketal or acetal, and 2,2-dimethyl-1-(2-nitrophenyl)propane-1,3-diol ketal or acetal. Other protecting groups well known in the art are also encompassed. Similarly, modified versions of natural amino acids are also intended to qualify as natural version of the amino acid for sequence identity purposes. For example, an amino acid comprising a side chain heteroatom which can be covalently modified (e.g., to add a conjugation handle, optionally through a linker), such as a lysine, glutamine, glutamic acid, asparagine, aspartic acid, cysteine, or tyrosine, which has been covalently modified would be counted as the base amino acid (see, e.g., Structure 2 below, which would be counted as a lysine for sequence identity and SEQ ID purposes). Similarly, an amino acid comprising another group added to the C or N-terminus would be counted as the base amino acid.

Referred to herein are amino acid or amino acid sequences which appear “upstream” of another referenced amino acid sequence. The term “upstream” in this context means the indicated amino acid or amino acid sequence is affixed to the N-terminal residue of the referenced amino acid sequence (e.g., a methionine residue positioned upstream of a sequence “SDGTK” (SEQ ID NO: 245) would have a sequence of “MSDGTK” (SEQ ID NO: 246)). In some cases, residue numbering of “upstream” amino acids or amino acid sequences uses a negative numbered numbering system (e.g., reference to positions at a −1, −2, or −3 position relative to a reference sequence). When such a numbering system is used, the −1 position corresponds to the amino acid affixed to the N-terminus of the reference sequence, the −2 position corresponds to the amino acid affixed at the N-terminus of the −1 position, and so on and so forth. For example, a sequence of AM positioned upstream of a reference sequence SDGTK (SEQ ID NO: 245) would result in a full sequence of AMSDGTK (SEQ ID NO: 247), where M is at the −1 position and A is at the −2 position relative to the reference sequence.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0, 2, 3, 4, or 4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal, which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

As used herein, the term “number average molecular weight” (Mn) means the statistical average molecular weight of all the individual units in a sample, and is defined by Formula (1):

$$Mn = \frac{\sum {N}_{i} {M}_{i}}{\sum {N}_{i}} \qquad Formula ( 1 )$$

where Mᵢ is the molecular weight of a unit and N is the number of units of that molecular weight.

As used herein, the term “weight average molecular weight” (Mw) means the number defined by Formula (2):

$$Mw = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g. mass spectrometry, size exclusion chromatography, dynamic light scattering, analytical centrifugation, etc.).

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g., terminal alkyne, substituted | triazole |
| | cyclooctyne (e.g., dibenzocycloocytne (DBCO), | |
| | difluorocyclooctyne, bicyclo[6.1.0]nonyne, etc.)) | |
| Phosphine | Azide/ester pair | amide |
| Tetrazine | trans-cyoclooctene | dihydropyridazine |
| Amine | Activated ester (e.g., N-hydroxysuccinimide | amide |
| | ester, pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., | amide |
| trifluoroborate | O-carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “PEG conjugation handle” is a conjugation handle attached to a PEG group (either directly or through a linker).

### II. Activatable IL-18 Polypeptides

The present disclosure relates to activatable IL-18 (Act-IL-18) polypeptides that are useful as therapeutic agents. Activatable IL-18 polypeptides provided herein can be used as immunotherapies or as parts of other immunotherapy regimens.

In one aspect, the disclosure relates to artificial moieties which are attached to an IL-18 polypeptide. An artificial moiety can comprise peptides, amino acids, and other groups such as polymers or spaces used to link the artificial moiety to the IL-18 polypeptide. The artificial moiety comprises a cleavable group which, when cleaved, releases all or a portion of the terminal moiety. Upon cleavage, the Act-IL-18 is converted into an active form of the IL-18 polypeptide which is capable of performing IL-18 activity.

In one aspect, provided herein, is an Act-IL-18 polypeptide comprising an artificial terminal moiety attached to an IL-18 polypeptide. In some embodiments, the artificial terminal moiety inhibits the Act-IL-18 polypeptide from interacting with and/or signaling through an IL-18 receptor. In some embodiments, the artificial terminal moiety is capable of undergoing a change in response to a condition or stimulus which results in a conversion of the Act-IL-18 polypeptide into an active IL-18 polypeptide. In some embodiments, the change is a cleavage of at least a portion of the artificial terminal moiety from the IL-18 polypeptide.

In one aspect, an Act-IL-18 of the instant disclosure is activated in or near a target tissue of a subject. In some embodiments, the Act-IL-18 is preferentially activated in or near the target tissue of a subject (e.g., activated at a higher rate in or near the target tissue compared to other tissue). In some embodiments, the Act-IL-18 is activated preferentially at or near a target tissue of the subject such that the area at or near the target tissue comprises at least 2-fold, at least 4-fold, at least 8-fold, at least 10-fold, at least 15-fold, at least 20-fold, at least 50-fold, at least 100-fold, at least 200-fold, at least 500-fold, or at least 1000-fold the concentration of the active form of the IL-18 polypeptide compared to non-target tissues. In some embodiments, the concentrations compared are the peak concentrations of the active form of the IL-18 polypeptide after administration of the Act-IL-18. In some embodiments, the Act-IL-18 is preferentially activated in or near disease tissue of the subject. In some embodiments, the Act-IL-18 polypeptide is preferentially activated in or near cancer tissue of the subject. In some embodiments, the Act-IL-18 polypeptide is preferentially activated in or near a tumor microenvironment. In some embodiments, the Act-IL-18 is preferentially activated in the tumor microenvironment compared to non-tumor tissue.

In one aspect, provided herein, is an activatable interleukin-18 (Act-IL-18) polypeptide comprising: an artificial terminal moiety attached to an interleukin-18 (IL-18) polypeptide, wherein the artificial terminal moiety comprises a specific cleavage site, and wherein cleavage at the specific cleavage site converts the Act-IL-18 into an active form of the IL-18 polypeptide.

### Artificial Moieties

In one aspect, provided herein, are artificial moieties attached to IL-18 polypeptides. Artificial moieties as provided herein serve to detune the activity of the IL-18 polypeptide while they are attached in an intact form. In some embodiments, cleavage of the artificial moiety serves to activate the IL-18 polypeptide (e.g., allowing the IL-18 polypeptide to signal through IL-18Rαβ). An artificial moiety provided herein can be attached to any residue. In some embodiments, the artificial moiety is an artificial terminal moiety (e.g., attached to a terminal residue of the IL-18 polypeptide).

In some embodiments, artificial moieties can have other functionalities attached (e.g., in addition to being attached to the IL-18 polypeptide, the artificial moiety is also attached to another group, such as an additional polypeptide (e.g., antibody, dummy receptor, another cytokine), a half-life extension polymer (e.g., poly(ethylene glycol) (PEG)), or another desired functionality. In some embodiments, cleavage of the artificial moiety serves also to cleave this additional group from the IL-18 polypeptide.

### Artificial Terminal Moieties

In one aspect, the Act-IL-18 polypeptides provided herein comprise an artificial terminal moiety. In some embodiments, the artificial terminal moiety is a functionality, such as a peptide, small molecule, or other group, which is covalently attached to an IL-18 polypeptide. In some embodiments, the artificial terminal moiety is a peptide. When the artificial terminal moiety is a peptide, it is in some cases alternatively referred to as an “artificial terminal peptide” herein. An artificial terminal moiety is a group which is not naturally attached to the terminus of a WT IL-18 polypepeptide, such as the natural precursor 36 amino acid propeptide directly attached to the WT IL-18. When the IL-18 polypeptide is a variant IL-18 polypeptide (e.g., having at least one amino acid substitution relative to WT IL-18), then the artificial terminal moiety can be the natural propeptide. In some embodiments, the artificial terminal peptide is engineered to possess the properties provided herein. In some embodiments, the artificial terminal moiety is fused to an IL-18 polypeptide (e.g., as a fusion protein). In some embodiments, the artificial terminal moiety is chemically attached to an IL-18 polypeptide, or is incorporated into an IL-18 polypeptide by synthetic means (e.g., during synthesis of an IL-18 polypeptide).

In some embodiments, an artificial terminal moiety provided herein inhibits at least one activity associated with an IL-18 polypeptide, such as the ability to bind to an IL-18 receptor or effectuate signaling through the IL-18 receptor (IL-18Rαβ) (e.g., inducing production of IFNγ in an immune cell). In some embodiments, when the artificial terminal moiety is intact, the Act-IL-18 polypeptide is in an inactive state (e.g., lacks or has a substantially diminished ability to bind IL-18Rαβ or signal through IL-18Rαβ).

In some embodiments, the presence of the intact artificial terminal moiety on the IL-18 polypeptide results in the Act-IL-18 polypeptide displaying a binding affinity to IL-18Rαβ or an IL-18R subunit which is at least 10-fold lower, at least 100-fold lower, at least 200-fold lower, at least 500-fold lower, or at least 1000-fold lower than WT IL-18. In some embodiments, the presence of the intact artificial terminal moiety on the IL-18 polypeptide results in the Act-IL-18 polypeptide displaying a binding affinity to IL-18Rαβ or an IL-18R subunit which is at least 10-fold lower, at least 100-fold lower, at least 200-fold lower, at least 500-fold lower, or at least 1000-fold lower than the IL-18 polypeptide without the artificial terminal moiety.

In some embodiments, the presence of the intact artificial terminal moiety on the IL-18 polypeptide results in the Act-IL-18 polypeptide displaying an ability to induce IFNg production in a cell (e.g., an immune cell such as an NK cell) which is at least 10-fold lower, at least 100-fold lower, at least 200-fold lower, at least 500-fold lower, or at least 1000-fold lower than WT IL-18.

In some embodiments, the presence of the intact artificial terminal moiety on the IL-18 polypeptide results in the Act-IL-18 polypeptide displaying an ability to induce IFNγ production in a cell (e.g., an immune cell such as an NK cell) which is at least 10-fold lower, at least 100-fold lower, at least 200-fold lower, at least 500-fold lower, or at least 1000-fold lower than the IL-18 polypeptide without the artificial terminal moiety.

In some embodiments, the artificial terminal moiety comprises a specific cleavage site. In some embodiments, the specific cleavage site is a site which is amenable to cleavage under certain specified or known conditions. Non-limiting examples of specific cleavage sites include protease cleavage sites, sites amenable to cleavage at certain pH ranges (e.g., acid labile bonds), sites amenable to cleavage via oxidation or reduction (e.g., disufulfide bonds), photocleavable linkers, and others.

In some embodiments, the specific cleavage site is selected such that it is preferentially cleaved (e.g., cleaved at a faster rate or cleaved in more abundance) at a designated target tissue of a subject. In some embodiments, the specific cleavage site is preferentially at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the target tissue is diseased tissue of the subject. In some embodiments, the target tissue is cancer tissue of the subject. In some embodiments, the target tissue is a tumor microenvironment. In some embodiments, the target tissue is a tumor.

In some embodiments, the specific cleavage site is positioned such that all or only a portion of the artificial terminal moiety is removed from the IL-18 polypeptide after cleavage. In some embodiments, none of the artificial terminal moiety is present on the IL-18 polypeptide after cleavage (e.g., only residues corresponding to residues in SEQ ID NO: 1 are present after cleavage). In some embodiments, a portion of the artificial terminal moiety remains attached to the IL-18 polypeptide after cleavage.

The artificial terminal moiety can be attached to either the N-terminal residue or the C-terminal residue of the IL-18 polypeptide. In some embodiments, the artificial terminal moiety is attached to the N-terminal residue. In some embodiments, the artificial terminal moiety is attached to the N-terminal amine of the IL-18 polypeptide. In some embodiments, the artificial terminal moiety is attached to the C-terminal residue. In some embodiments, the artificial terminal moiety is attached to the C-terminal carboxyl of the IL-18 polypeptide. In some embodiments, the N-terminal residue is the residue closest to residue position 1 of SEQ ID NO: 1 which is present on an IL-18 polypeptide as provided herein (e.g., the first residue of SEQ ID NO: 1 which has not been truncated). In some embodiments, the N-terminal residue of the IL-18 polypeptide is the residue at a position corresponding to position 1 in SEQ ID NO: 1. In some embodiments, the N-terminal residue of the IL-18 polypeptide is Y1. In some embodiments, the N-terminal residue of the IL-18 polypeptide is Y1G. In some embodiments, the C-terminal residue is the residue at position 157 of SEQ ID NO: 1. In some embodiments, the C-terminal residue is D157.

In some embodiments, terminal residues of the IL-18 polypeptide are substituted such that the artificial terminal moiety is positioned such that the entirety of the artificial terminal moiety is cleaved from the IL-18 polypeptide. For example, if it is intended to introduce a cleavage site at a position corresponding to residue 1 of SEQ ID NO: 1, a protease cleavage sequence P₁-P₂-P₃-P′₁-P′₂-P′₃ can be selected (where the cleavage site is between P₃ and P′₁), and residues 1, 2, and 3 of SEQ ID NO: 1 can be substituted for P′₁, P′₂, and P′₃ respectively, with P₁-P₂-P₃- appended thereon. In this case, the artificial terminal moiety would be considered to comprise P₁-P₂-P₃-, with P′₁, P′₂, and P′₃ as part of the IL-18 polypeptide (substituted residues).

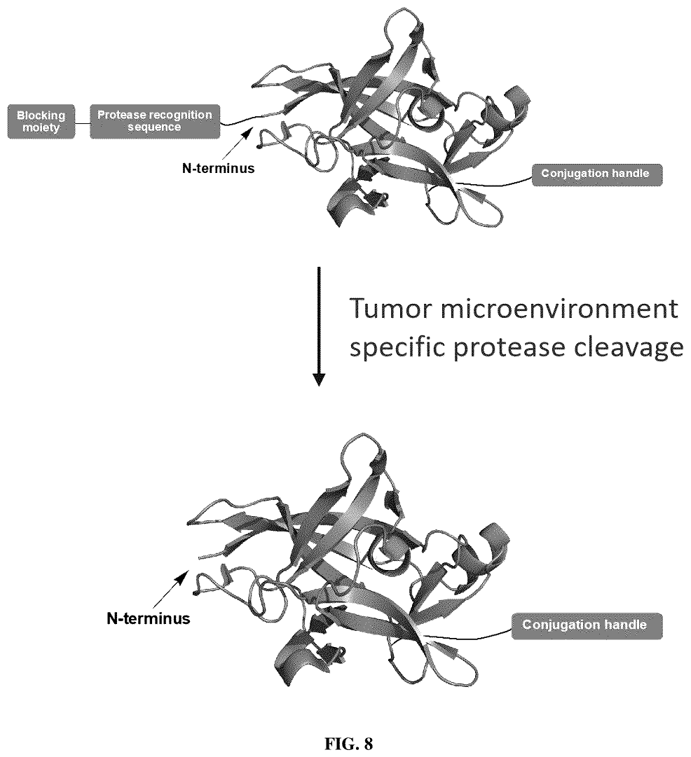

Examples of artificial terminal moieties and their mechanisms of action are shown in FIGS. 8-13. FIG. 8 depicts an artificial terminal peptide affixed to a terminal residue of the IL-18 polypeptide. Also attached to the artificial moiety is a blocking moiety which facilitates keeping the IL-18 polypeptide in an inactive state. When the artificial terminal moiety is cleaved (e.g., by a tumor microenvironment protease), the entire artificial terminal moiety is cleaved (revealing the terminal residue of the IL-18 polypeptide, as is the blocking moiety. The IL-18 in this example has a conjugation handle attached elsewhere to the IL-18 polypeptide, which can serve to attach an additional group (e.g., half-life extension polymer or an additional polypeptide), and it can be attached either before or after cleavage of the artificial terminal moiety.

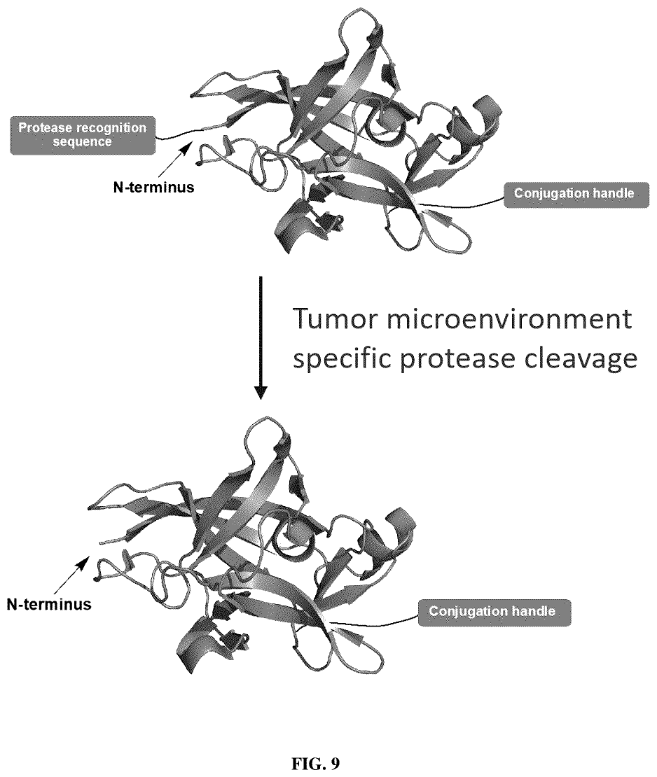

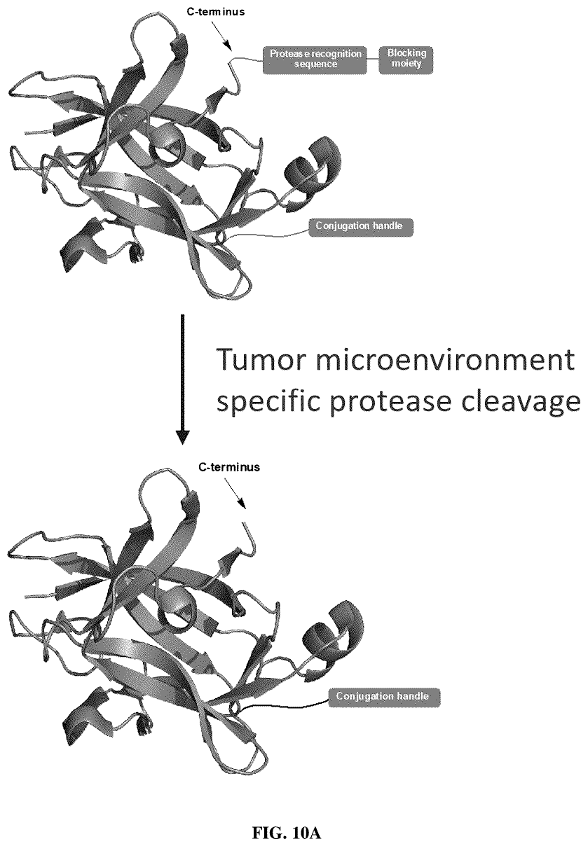

FIG. 9 shows a similar Act-IL-18 construct shown in in the previous figure, but lacks the blocking moiety. In some instances, the presence of only a short peptide on the N-terminus can sufficiently detune the activity of the IL-18 polypeptide, and the activity can be restored upon cleavage.

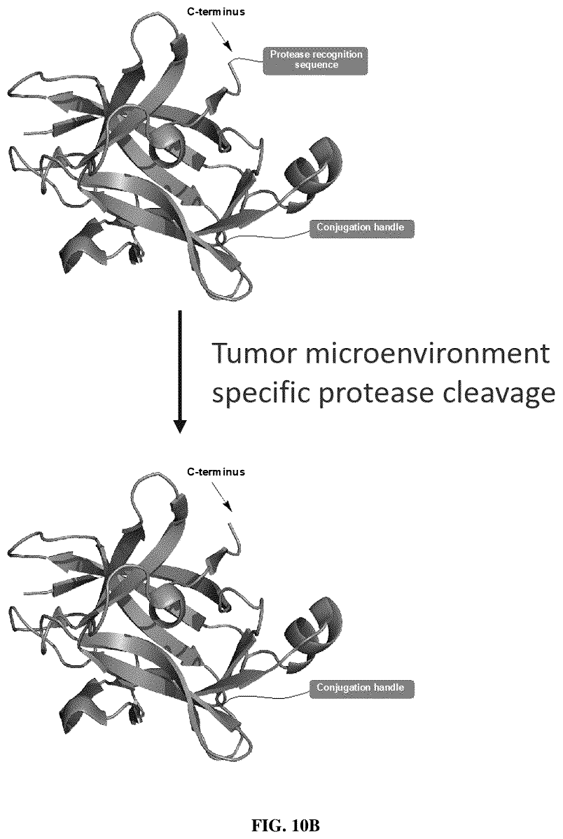

FIG. 10A shows a similar Act-IL-18 construct to that shown in FIG. 8, but the artificial terminal moiety is attached to the C-terminus.

FIG. 10B shows a similar Act-IL-18 construct to that shown in FIG. 9, but the artificial terminal moiety is attached to the C-terminus.

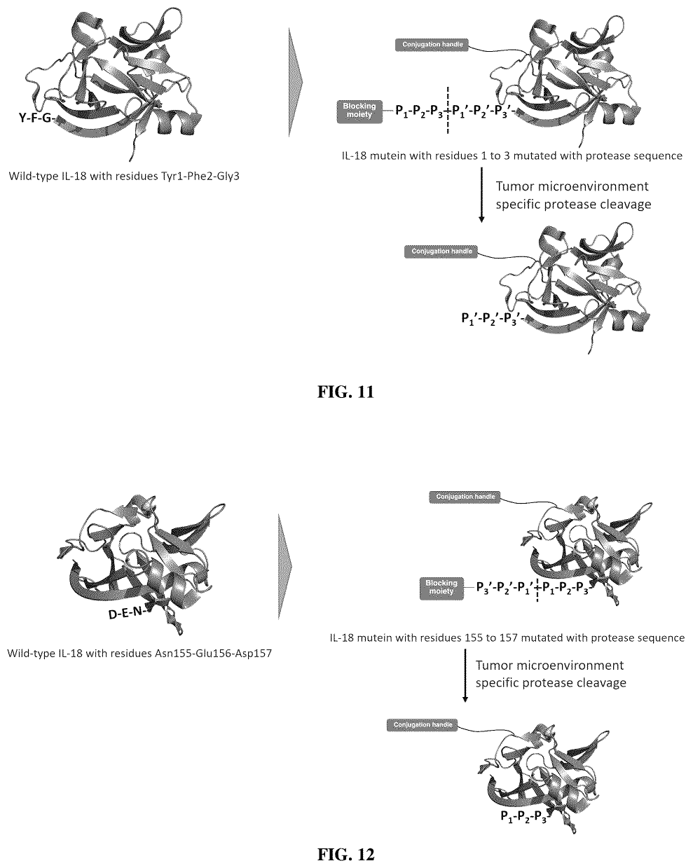

FIG. 11 depicts an analogous construct to that shown in FIG. 8, but residues 1, 2, and 3 of the IL-18 polypeptide are substituted such that the artificial terminal moiety is cleaved entirely (residues 1, 2, and 3, though substituted, still correspond to positions 1, 2 and 3 of SEQ ID NO: 1). FIG. 12 is analogous to FIG. 11, but the artificial terminal moiety is at the C-terminus. Alternatively to substituting residues 1, 2, and 3 of the IL-18 as depicted in FIG. 11, a cleavable peptide could be selected such that natural residues 1, 2, and 3 of the IL-18 polypeptide (e.g., Y, F and G, respectively), or a subset of the natural residues, form part of the cleavage recognition site of the relevant protease such that the entire artificial terminal moiety is cleaved, thus leaving an unmodified N-terminus of the IL-18 polypeptide after cleavage. For example, a sequence of -PLG- can be appended the N-terminal Y of the IL-18 polypeptide to allow complete cleavage of the artificial terminal moiety by a matrix metalloprotease. Such an approach could also be adopted at the C-terminus of the IL-18 polypeptide.

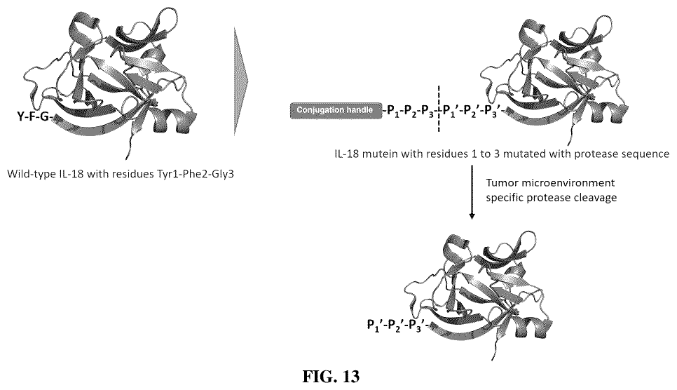

FIG. 13 depicts an Act-IL-18 polypeptide comprising an artificial terminal moiety which has a conjugation handle attached to it such that cleavage of the artificial terminal moiety will also cleave the conjugation handle (or anything which has been attached to the conjugation handle) from the IL-18 polypeptide. By way of non-limiting example, such a construct could be used to conjugate an antibody to the IL-18 polypeptide. Such a construct could be used to tissue specifically deliver an IL-18 polypeptide, then also activate it at the desired site through cleavage at the target tissue.

### Peptide Artificial Terminal Moieties

In some embodiments, the artificial terminal moiety comprises a peptide (a.k.a. “artificial terminal peptide”). In some embodiments, the artificial terminal moiety consists of a peptide.

In some embodiments, cleavage of the specific cleavage site leaves no amino acid residues attached to the IL-18 polypeptide. In some embodiments, cleavage of the specific cleavage site leaves at least 1 amino acid residue attached to the IL-18 polypeptide. In some embodiments, cleavage of the specific cleavage site leaves at most 1, 2, 3, 4, or 5 amino acid residues attached to the IL-18 polypeptide. In some embodiments, cleavage of the specific cleavage site leaves 1, 2, 3, 4, or 5 amino acid residues attached to the IL-18 polypeptide. In some embodiments, cleavage of the cleavage site leaves 1, 2, 3, 4, 5, 6, 7, 8, 9, or 10 or more amino acid residues attached to the IL-18 polypeptide.

In some embodiments, an Act-IL-18 can optionally contain a tag used for expression and/or purification of a recombinant Act-IL-18 as provided herein. The tag can be situated upstream of an N-terminal artificial terminal peptide or downstream of a C-terminal artificial terminal peptide. The tag may be removed (e.g., enzymatically) prior to final formulation and/or administration to a subject. Exemplary tags include a HIS6 (HHHHHH, SEQ ID NO: 84), a Strep Tag (WSHPQFEK (SEQ ID NO: 85) or AWAHPQPGG (SEQ ID NO: 86), or a chitin binding tag

TTNPGVSAWQVNTAYTAGQLVTYNGKTYKCLQPHTSLAGWEPSNVPALW
QLQ, SEQ ID NO: 87).

### Protease Cleavable Artificial Terminal Moieties

In some embodiments, the artificial terminal moiety can be cleaved by a protease (e.g., the artificial terminal moiety comprises a cleavable peptide). In some embodiments, the artificial terminal moiety contains a site of cleavage that can be cleaved specifically by one or more proteases. In some embodiments, the artificial terminal moiety contains a site of cleavage that can be cleaved at a site preferred by one or more proteases. In some embodiments, the specific cleavage site is a protease cleavage site.

In some embodiments, the protease is found at higher concentrations and/or demonstrates higher proteolytic activity at or near a target tissue of a subject. In some embodiments, the target tissue is disease tissue. In some embodiments, the target tissue is a cancer. In some embodiments, the target tissue is a tumor microenvironment.

In some embodiments, the protease is found at higher concentrations and/or demonstrates higher proteolytic activity at or near the tumor microenvironment relative to non-tumor tissue. In some embodiments, the protease is found at higher concentrations at or near the tumor microenvironment relative to non-tumor tissue. In some embodiments, the protease demonstrates higher proteolytic activity at or near the tumor microenvironment relative to non-tumor tissue. In some embodiments, the protease is selected from kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, and a tumor cell surface protease. In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the protease is selected from kallikrein, thrombin, chymase, carboxypeptidase A, an elastase, proteinase 3 (PR-3), granzyme M, urokinase plasminogen activator (uPA), a calpain, a matrix metalloproteinase (MMP), a disintegrin and metalloproteinase (ADAM), a fibroblast activation protein alpha (FAP), a matriptase, a plasminogen activator, a cathepsin, a caspase, a tryptase, and a tumor cell surface protease. In some embodiments, the cleavable peptide is cleavable by a protease selected from a kallikrein, thrombin, chymase, carboxypeptidase A, an elastase, proteinase 3 (PR-3), urokinase plasminogen activator (uPA), granzyme M, a calpain, a matrix metalloproteinase (MMP), a disintegrin and metalloproteinase (ADAM), a fibroblast activation protein alpha (FAP), a matriptase, a plasminogen activator, a cathepsin, a caspase, a tryptase, a matriptase, and a tumor cell surface protease, or any combination thereof. In some embodiments, the protease is urokinase plasminogen activator (uPA) or matriptase. In some embodiments, the cleavable peptide is cleavable by urokinase plasminogen activator (uPA) or matriptase. In some embodiments, the protease is a protease selected from Table 1 or Table 2A. In some embodiments, the protease is a protease selected from Table 2B.

TABLE 1
Tissue/Tumor specific proteases

| | | |
|---|---|---|
| Protease | Specificity | Other Aspects |

Secreted by CD8⁺ Cytotoxic T cells

| | | |
|---|---|---|
| Granzyme B (grB) | Cleaves after Asp | Type of serine protease; Strongly |
| | | implicated in inducing perforin-dependent |
| | | target cell apoptosis |
| Granzyme A (grA) | trypsin-like, cleaves after basic | Type of serine protease. |
| | residues | |
| Granzyme H (grH) | Unknown substrate specificity | Type of serine protease; |
| | | Other granzymes are also secreted by killer |
| | | T cells, but not all are present in humans |
| Caspase-8 | Cleaves after Asp residue | Type of cysteine protease; plays and |
| | | essential role in TCR-induced cellular |
| | | expansion. |
| Mucosa-associated | Cleaves after Arg residues | Type of cysteine protease; likely acts both |
| lymphoid tissue | | as a scaffold and proteolytically active |
| (MALT1) | | enzyme in the CBM-dependent signaling |
| | | pathway. |
| Tryptase | Target: angiotensin I, | Type of mast cell-specific serine protease; |
| | fibrinogen, prourokinase, | trypsin-like; resistant to inhibition by |
| | TGFβ; preferentially cleaves | macromolecular protease inhibitors |
| | proteins after lysine or | expressed in mammals due to their |
| | arginine residues. | tetrameric structure, with all sites facing |
| | | narrow central pore; also associated with |
| | | inflammation. |

Associated with Inflammation:

| | | |
|---|---|---|
| Thrombin | Targets: FGF-2 | Type of serine protease; modulates activity |
| | HB-EGF, Osteo-pontin, | of vascular growth factors, chemokines |
| | PDGF, VEGF | and extracellular proteins; strengthens |
| | | VEGF-induced proliferation; induces cell |
| | | migration; angiogenic factor; regulates |
| | | hemostasis |
| Chymase | Exhibit chymotrypsin-like | Type of mast cell-specific serine protease. |
| | specificity, cleaving proteins | |
| | after aromatic amino acid | |
| | residues. | |
| Carboxypeptidase A | Cleaves amino acid residues | Type of zinc-dependent metalloproteinase |
| (MC-CPA) | from C-terminal end of | |
| | peptides and proteins | |
| Kallikreins | Targets: high molecular | Type of serine protease; modulate |
| | weight kininogen, pro- | relaxation response; contribute to |
| | urokinase | inflammatory response; activates pro- |
| | | apoptotic signaling |
| Elastase | Targets: E-cadherin, GM-CSF, | Type of neutrophil serine protease; |
| | IL-1, IL-2, IL-6, IL-8, | degrades ECM components; regulates |
| | p38^{MAPK}, TNFα, VE-cadherin. | inflammatory response; activates pro- |
| | | apoptotic signaling. |
| Cathepsin G | Targets: ENA-78, IL-8, | Type of serine protease; degrades ECM |
| | MCP-1, MMP-2, MT1-MMP, | components; chemo-attractant of |
| | PAI-1, RANTES, TGFβ, TNFα | leukocytes; regulates inflammatory |
| | | response; promotes apoptosis. |
| PR-3 | Targets: ENA-78, IL-8, IL-18, | Type of serine protease; promotes |
| | JNK, p38^{MAPK}, TNFα | inflammatory response; activates pro- |
| | | apoptotic signaling. |
| Granzyme M (GrM) | Cleaves after Met and other | Type of serine protease; only expressed in |
| | long, unbranched hydrophobic | NK cells. |
| | residue. | |
| Calpains | Cleave between Arg and Gly | Family of cysteine proteases; calcium- |
| | | dependent; activation is involved in the |
| | | process of numerous inflammation- |
| | | associated diseases. |

In some embodiments, the protease cleavage site is comprised within a recognition sequence recognized by the protease. In some embodiments, the recognition sequence is a natural peptide sequence which has been incorporated into the artificial terminal moiety. In some embodiments, the recognition sequence is a synthetic (e.g., man-made, designed, or engineered) sequence. In some embodiments, the recognition sequence comprises an amino acid sequence having at least about 80%, at least about 85%, at least about 90%, at least about 95%, at least about 96%, at least about 97%, at least about 98%, at least about 99%, or 100% identical to a sequence set forth in Table 2A.

TABLE 2A
Protease specific peptide recognition sequences

| SEQ | | Protein |
|---|---|---|
| ID | | Sequence |
| NO: | Protease | Cleaved |
| 201 | MMP7 | KRALGLPG |
| 202 | MMP9 | PR(S/T)(L/I)(S/T) |
| 203 | MMP9 | LEATA |
| 204 | MMP11 | GGAANLVRGG |
| 205 | MMP14 | SGRIGFLRTA |
| 206 | MMP | PLGLAG |
| 207 | MMP | PLGLAX |
| 208 | MMP | ESPAYYTA |
| 209 | MMP | RLQLKL |
| 210 | MMP | RLQLKAC |
| 211 | Urokinase plasminogen | SGRSA |
| | activator (uPA) | |
| 212 | uPA | DAFK |
| 213 | uPA | GGGRR |
| 214 | Lysosomal Enzyme | GFLG |
| 215 | Lysosomal Enzyme | ALAL |
| 216 | Lysosomal Enzyme | FK |
| 217 | CathepsinB | NLL |
| 218 | CathepsinD | GLLYA |
| 219 | Cathepsin K | GGPRGLPG |
| 220 | Prostate Specific | HSSKLQ |
| | Antigen | |
| 221 | Prostate Specific | HSSKLQL |
| | Antigen | |
| 222 | Prostate Specific | HSSKLQEDA |
| | Antigen | |
| 223 | Thrombin | DPRSFL |
| 224 | Thrombin | PPRSFL |
| 225 | Caspase-3 | DEVD |
| 226 | Caspase-3 | DEVDP |
| 227 | Caspase-3 | KGSGDVEG |
| 228 | Interleukin 1P | GWEHDG |
| | converting enzyme | |
| 229 | FAP | KQEQNPGST |
| 230 | Kallikrein 2 | GKAFRR |
| 231 | Plasmin | DAFK |
| 232 | Plasmin | DVLK |
| 233 | Plasmin | DAFK |
| 234 | TOP | ALLLALL |

In some embodiments, the recognition sequence comprises an amino acid sequence having at least about 80%, at least about 85%, at least about 90%, at least about 95%, at least about 96%, at least about 97%, at least about 98%, at least about 99%, or 100% identical to a sequence set forth in Table 2B.

TABLE 2B
Additional specific peptide recognition
sequences

| Sequence | Cleavable Peptide | |
|---|---|---|
| Number | Sequence | Protease |
| 100 | SGGPGPAGMKGLPG | MMP9 (also |
| | | referred to |
| | | herein as |
| | | Linker X) |
| 101 | SGGPGPAGMKGLPGS | MMP9 |
| 102 | EAGRSANHEPLGLVAT | MMP2-7-9-14 + |
| | | matriptase + |
| | | uPA + Legumain |
| 103 | PQASTGRSGG | MMP9 + |
| | | matriptase + |
| | | uPA |
| 104 | PQGSTGRAAG | MMP9 + |
| | | matriptase + |
| | | uPA |
| 105 | PPASSGRAGG | MMP9 + |
| | | matriptase + |
| | | uPA |
| 106 | RSGVPLSLYSGSGGGK | MMP7-9-10 |
| 107 | RSGMPYDLYHPSGK | MMP7-9-10 |
| 108 | RGPDSGGFMLTSGK | MMP7-9-10 |
| 109 | RGSGHEQLTVSGGSK | MMP7-9-10 |
| 110 | RSGRAAAVKSPSGK | MMP7-9-10 |
| 111 | RGSGISSGLLSGRSDNHSGK | MMP7-9-10 |
| 112 | RGDLLAVVAASGGK | MMP7-9-10 |
| 113 | RGGISSGLLSGRSGK | MMP7-9-10 |
| 114 | SGGGKKLADEPEGGS | Meprin A/B |
| 115 | GGGKFLADEPEGG | Meprin A/B |
| | | (High |
| | | Efficiency) |
| 116 | ARLQSAAP | Cathepsin |
| | | S, K, L |
| 117 | ARLQSAAPAGLKGA | Cathepsin |
| | | S + MMP9 + |
| | | Meprin A |
| 118 | GSGGPGPAGMHGLPGGS | MMP9 (High |
| | | Efficiency) |
| 119 | GGGSHTGRSAYFGGGS | uPA |
| 120 | SGGPGPAGLKGAPGS | MMP9-2 |
| 121 | VPLSLYSG | MMP2-7-9 |
| 122 | SGLLSGRSDNH | uPA + |
| | | Matriptase + |
| | | legumain |
| 123 | SGRSDNIGGGS | uPA |
| 124 | LQESLRSKESGRSDI | MMP-14 + uPA |
| 125 | ISSGLLSGRSDNH | uPA |
| 126 | LSGRSDDH | uPA |
| 127 | ISSGLLSGRSDQH | uPA |
| 128 | ISSGLLSGRSDNI | uPA |
| 129 | PLGLAG | MMP2-7-9 |
| 130 | GPAGMKGL | MMP |
| 131 | LSGRSDQH | MMP |
| 132 | LSGRSDNI | MMP |
| 133 | ISSGLLSGRSDNH | MMP |
| 134 | GPLGVRG | MMP |
| 135 | GPLGLAR | MMP |
| 136 | GPAALVGA | MMP |
| 137 | GPAALIGG | MMP |
| 138 | GPLNLVGR | MMP |
| 139 | GPAGLVAD | MMP |
| 140 | GPANLVAP | MMP |
| 141 | VPLSLYSG | MMP |
| 142 | SGESPAYYTA | MMP |
| 143 | GGPRGLG | MMP |
| 144 | HSSKLQ | MMP |
| 145 | HSSKLQL | MMP |
| 146 | KRALGLPG | MMP7 |
| 147 | LEATA | MMP9 |
| 148 | GGAANLVRGG | MMP11 |
| 149 | SGRIGFLRTA | MMP14 |
| 150 | PLGLA | MMP |
| 151 | ESPAYYTA | MMP |
| 152 | RLQLKL | MMP |
| 153 | RLQLKAC | MMP |
| 154 | SGRSA | uPA |
| 155 | (D-Ala)FK | uPA |
| 156 | GGGRR | uPA |
| 157 | GFLG | Lysosomal |
| | | Enzyme |
| 158 | HSSKLQEDA | Prostate |
| | | Specific |
| | | Antigen |
| 159 | LVLASSSFGY | HSV Protease |
| 160 | GVSQNYPIVG | HIV Protease |
| 161 | GVVQASCRLA | CMV Protease |
| 162 | DPRSFL | Thrombin |
| 163 | PPRSFL | Thrombin |
| 164 | DEVD | Caspase-3 |
| 165 | DEVDP | Caspase-3 |
| 166 | KGSGDVEG | Caspase-3 |
| 167 | GWEHDG | Interleukin 1ß |
| | | converting |
| | | enzyme |
| 168 | EDDDDKA | Enterokinase |
| 169 | KQEQNPGST | FAP |
| 170 | GKAFRR | Kallikrein 2 |
| 171 | DAFK | Plasmin |
| 172 | DVLK | Plasmin |
| 173 | DAFK | Plasmin |
| 174 | ALLLALL | TOP |
| 175 | SGAKPRALTA | MMP A3 |
| 176 | SGLRLAAITA | MMP B49 |
| 177 | SGESLAYYTA | MMP B74 |
| 178 | SGESPAYYTA | MMP B74P |
| 179 | SGESLRYYTA | MMP B74R |
| 180 | SGRSLSRLTA | MMP C9 |
| 181 | SGRSLRRLTA | MMP C9R |
| 182 | SGAVSWLLTA | MMP A13 |
| 183 | SGAPSWLLTA | MMP A13P |
| 184 | SGAVRWLLTA | MMP A13R |
| 185 | SGANISDLTA | MMP B37 |
| 186 | SGNRYSSLTA | MMP A34 |
| 187 | SGHMHKALTA | MMP A21/MMPA21A |
| 190 | GEEGEEPLGLAG | MMP |
| 191 | GPLGLAG | MMP |
| 192 | EAGRSANHTPAGLTGP | |
| 193 | GEAGRSANHTPAGLTGP | |
| 194 | PLG | MMP (partial |
| | | sequence) |

In some embodiments, cleavage of the specific cleavage site leaves no amino acid residues attached to the IL-18 polypeptide. In such cases, the protease recognition sequence can be selected such that portions of the IL-18 polypeptide make up part of the recognition sequence, or are compatible therewith. For example, in some embodiments, the sequence PLG is appended to the N-terminus of the IL-18 polypeptide (e.g., residue 1 of SEQ ID NO: 1), which results in the specific cleavage site being between the G of the PLG and the N-terminus of the IL-18 polypeptide, thereby resulting in a “scarless” activated IL-18 polypeptide after cleavage.

In some embodiments, a portion of the protease recognition sequence which defines the specific cleavage site will be comprised in the sequence of the IL-18 polypeptide (e.g., part of the protease recognition sequence will be comprised at positions which correspond to positions of SEQ ID NO: 1). In some embodiments, the portion of the protease recognition sequence comprised in the sequence of the IL-18 polypeptide will be substituted relative to the sequence set forth in SEQ ID NO: 1. For example, in some embodiments, the last three amino acids of SEQ ID NO: 1 are substituted with -PLG in order to form part of a protease recognition site with the artificial terminal moiety.

In some embodiments, cleavage of the specific cleavage site leaves at least 1 amino acid residue attached to the IL-18 polypeptide. In some embodiments, cleavage of the specific cleavage site leaves at most 1, 2, 3, 4, or 5 amino acid residues attached to the IL-18 polypeptide. In some embodiments, cleavage of the specific cleavage site leaves 1, 2, 3, 4, or 5 amino acid residues attached to the IL-18 polypeptide. In some embodiments, cleavage of the cleavage site leaves 1, 2, 3, 4, 5, 6, 7, 8, 9, or 10 or more amino acid residues attached to the IL-18 polypeptide. In some embodiments, cleavage of the specific cleavage site leaves at most 10, 9, 8, 7, 6, 5, 4, 3, 2, or 1 amino acid residues attached to the IL-18 polypeptide.

Further exemplary cleavable peptide sequences which can be incorporated into an Act-IL-18 polypeptide as provided herein can be found in any one of U.S. Patent Publication Nos: US2010/0189651, US2016/0289324, US2018/0125988, US2019/0153115, US2020/0385469, US2021/0260163, US2022/0048949, US2022/0267400, US2021/0115102, US2022/0002370, US2021/0163562, US20200392235, US2021/0139553, US2021/0317177, US2020/0283489, US2021/0002343, US2021/0292421, US2021/0284728, US2021/0269530, US2022/0054544, US2021/0355219, US2022/0073613, US2021/0047406, and/or Patent Cooperation Treaty Publication Nos: WO2021/202675, WO2021/062406, WO2021/142471, WO2021/216468, WO2021/119516, WO2021/253360, WO2021/146455, WO2021/202678, WO2021/202673, WO2021/189139, WO2020/232303, WO2022/115865, WO2021/202678, and/or Chen et. al., J Bio Chem, 277, V6 P4485-4491 (2002).

### Redox Cleavable Artificial Terminal Moieties

In some instances, different tissues of a subject can exhibit different redox potentials depending on the activity of the tissue. For example, tumors and tumor microenvironments are associated with having substantially greater reduction potentials than other healthy tissues. Thus, in some embodiments, artificial terminal moieties provided herein utilize this property to allow preferential cleavage and activation of the IL-18 polypeptide at the tissue site.

In some embodiments, the artificial terminal moiety can be cleaved by a reduction or oxidation reaction. In some embodiments, the artificial terminal moiety contains a site of cleavage that can be cleaved specifically by a reduction or oxidation reaction. In some embodiments, the artificial terminal moiety contains a site of cleavage that can be cleaved at a site preferred by a reduction or oxidation reaction. In some embodiments, the specific cleavage site is a redox sensitive cleavage site.

In some embodiments, the redox sensitive cleavage site is preferentially cleaved at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the redox cleavage site is preferentially cleaved in a reducing environment. In some embodiments, the redox cleavage site is preferentially cleaved in a reducing environment relative to blood. In some embodiments, the redox cleavage site is preferentially cleaved in a reducing environment relative to interstitial fluids. In some embodiments, the redox cleavage site is preferentially cleaved in a reducing environment relative to lymphatic fluid. In some embodiments, the redox cleavage site is preferentially cleaved in a reducing environment of a tumor microenvironment.

### pH Cleavable Artificial Terminal Moieties

The pH of circulating blood is buffered in a narrow range of between 7.31 to 7.45. Variances outside this range typically result in acidosis or alkalosis, which are serious medical conditions. The tumor microenvironment is characteristically more acidic than circulating blood pH due to a metabolic dysregulation in tumor cells known as the Warburg effect: Growing tumor cells demonstrate a high rate of glycolysis followed by fermentation of pyruvate to lactic acid in the cytoplasm rather than oxidation of pyruvate in the mitochondrial TCA cycle. To maintain the pH of their cytoplasm, tumor cells transport hydrogen ions to the extracellular environment, resulting in an acidic tumor microenvironment.

In some embodiments, the specific cleavage site is a pH sensitive cleavage site. In some embodiments, the pH sensitive cleavage site is selected to preferentially cleave at a target tissue. In some embodiments, the target tissue is associated with a certain pH or a difference in pH compared to other local tissues.

In some embodiments, the pH sensitive cleavage site is cleaved at a pH below physiological blood pH (e.g., below about 7.3). In some embodiments, the pH sensitive cleavage site is preferentially cleaved at a pH below 7.3, below 7.2, below 7.1, or below 7.0. In some embodiments, the pH sensitive cleavage site is preferentially cleaved at acidic pH. In some embodiments, the pH sensitive cleavage site is preferentially cleaved at a pH of below 7, 6.5, 6, 5.5, or 5.

In some embodiments, the pH sensitive cleavage site is preferentially cleaved at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the tissue is a tumor microenvironment.

### Blocking Moieties

In some embodiments, the artificial terminal moiety comprises a blocking moiety. In some embodiments, the blocking moiety is a group which, when attached to the IL-18 polypeptide in the Act-IL-18 polypeptide, acts to disrupt or inhibit binding of the IL-18 polypeptide with the IL-18 receptor or a subunit thereof (e.g., as measured by experiments designed to detect binding, or by in vitro or in vivo activity analysis of the Act-IL-18 polypeptide).

In some embodiments, the blocking moiety is a steric blocking group or a specific blocking group. In some embodiments, the blocking moiety is a steric blocking group. In some embodiments, a steric blocking group has no specific interaction with the IL-18 polypeptide, but its presence hinders the interaction of the Act-IL-18 polypeptide with the receptor owing to its bulk. In some embodiments, the steric blocking group is a polymer (e.g., polyethylene glycol) or a polypeptide (e.g., albumin, an Fc region, etc.).

In some embodiments, the blocking moiety is a specific blocking group. In some embodiments, the specific blocking group has a specific binding or other interaction to IL-18. Non-limiting examples of specific blocking groups can include IL-18 propeptides, antibodies or antigen binding fragments which bind IL-18, IL-18 receptor subunits or domains or other fragments thereof, IL-18 binding proteins or fragments thereof, or other groups capable of specific binding to IL-18.

In some embodiments, the blocking moiety is a propeptide of IL-18. Endogenously, human IL-18 is expressed as an immature, inactive 193 amino acid having the sequence MAAEPVEDNCINFVAMKFIDNTLYFIAEDDENLESDYFGKLESKLSVIRNLNDQVLFIDQ GNRPLFEDMTDSDCRDNAPRTIFIISMYKDSQPRGMAVTISVKCEKISTLSCENKIISFKE MNPPDNIKDTKSDIIFFQRSVPGHDNKMQFESSSYEGYFLACEKERDLFKLILKKEDELG DRSIMFTVQNED (SEQ ID NO: 88), the first 36 amino acids of which are a propetide which is cleaved by caspases to yield the mature, active form of IL-18 (SEQ ID NO: 1). In some embodiments, the IL-18 propeptide blocking moiety is attached to the N-terminus of the IL-18 polypeptide (e.g., through a cleavable peptide comprising the specific cleavage site and any optional linker peptides) and In some embodiments, this propeptide or variants thereof is incorporated into an Act-IL-18 as a blocking moiety. In some embodiments, the blocking moiety is a human IL-18 propeptide or a variant thereof. In some embodiments, the blocking moiety is an IL-18 propeptide an amino acid sequence having at least 80%, 85%, 90%, 95%, 96%, 97%, 98%, 99%, or 100% sequence identity to the sequence set forth in SEQ ID NO: 89. In some embodiments, the blocking moiety is an IL-18 propeptide having a sequence at least 95% identical to the sequence set forth in SEQ ID NO: 89. In some embodiments, the blocking moiety is an IL-18 propeptide comprising the sequence set forth in SEQ ID NO: 89. In some embodiments, the blocking moiety is an IL-18 propeptide comprising the sequence set forth in SEQ ID NO: 89 with a substitution for residue C10 (e.g., a C10S substitution (SEQ ID NO: 90) or a C10A substitution (SEQ ID NO: 91). In some embodiments, the IL-18 propeptide comprises one or more modifications (e.g., amino acid substitutions) which reduce the affinity of the IL-18 propeptide for the IL-18 polypeptide in the Act-IL-18 polypeptide. In some embodiments where the blocking moiety is an IL-18 propeptide, the specific cleavage site of the Act-IL-18 polypeptide is different from that of the endogenous propeptide (i.e., the specific cleavage site is not the bond between D36 and Y37 of SEQ ID NO: 88). In some embodiments, the IL-18 propeptide acting as a blocking moiety is connected to the N-terminus of the IL-18 polypeptide in the Act-IL-18 polypeptide through another protease recognition sequence (and optionally one or more linking peptides).

In some embodiments, the blocking moiety is a modified IL-18 propeptide (e.g., human IL-18 propeptide) directly attached to the N-terminus of the IL-18 polypeptide. In some embodiments, the modified IL-18 propeptide comprises modifications which change the natural caspase cleavage site of SEQ ID NO: 88 to a site cleaved by another protease (e.g., any of the proteases provided herein, such as a matrix metalloprotease). In some embodiments, the three C-terminal amino acids of the IL-18 propaptide are substituted to -PLG. In some embodiments, the IL-18 propeptide comprises the -PLG substitution and

### Exemplary IL-18 Propeptide Sequences

| SEQ ID | Blocking Moiety Sequence |
|---|---|
| 89 | MAAEPVEDNCINFVAMKFIDNTLYFIAEDDENLESD |
| 90 | MAAEPVEDNSINFVAMKFIDNTLYFIAEDDENLESD |
| 91 | MAAEPVEDNAINFVAMKFIDNTLYFIAEDDENLESD |
| 92 | MAAEPVEDNCINFVAMKFIDNTLYFIAEDDENLPLG |

In some embodiments, an Act-IL-18 polypeptide comprising a human IL-18 propeptide or variant thereof as a blocking moiety exhibits substantially reduced activity compared to the IL-18 polypeptide by itself or the activated form of the IL-18 polypeptide (e.g., substantially no activity, or activity which is reduced by more than 1000-fold). In some embodiments, it may be desirable for the Act-IL-18 with an IL-18 propeptide attached to retain a greater activity prior to activation. In order to accomplish this, in some embodiments, it may be advantageous to use an IL-18 propeptide from a non-human species. In some embodiments, an Act-IL-18 polypeptide provided herein comprises an IL-18 propeptide from a non-human species. In some embodiments, the non-human species is a mammal. In some embodiments, the non-human species is a primate, a rodent, an equine, a bovine, an urcine, a porcine, an equine, a chiroptera, a camelid, or other animal. In some embodiments, the non-human IL-18 propeptide has an amino acid sequence which is at least 50%, 60%, 70%, or 75% identical to that of SEQ ID NO: 89.

In some embodiments, the blocking moiety comprises a portion (e.g., a domain or portion thereof) of an IL-18 receptor subunit, or a variant thereof. In some embodiments, the portion of the IL-18 receptor subunit or variant thereof is attached to the C-terminus of the IL-18 polypeptide in the Act-IL-18 polypeptide (e.g., through a cleavable peptide comprising the specific cleavage site and any optional linker peptides). In some embodiments, the blocking moiety comprises a portion of the IL-18 receptor alpha subunit or the IL-18 receptor beta subunit, or a variant thereof. In some embodiments, the blocking moiety comprises a portion of the IL-18 receptor alpha subunit or a variant thereof. In some embodiments, the blocking moiety comprises a domain of the IL-18 receptor alpha subunit or a variant thereof. In some embodiments, the blocking moiety comprises an extracellular domain, or a variant thereof, of the IL-18 receptor alpha subunit. In some embodiments, the blocking moiety comprises the D1, D2, or D3 domain, or a variant thereof, of the IL-18 receptor alpha subunit.

In some embodiments, the blocking moiety comprises the D3 domain, or a variant thereof, of the IL-18 receptor alpha subunit. The sequence of the human D3 domain of the IL-18 receptor alpha subunit is shown in SEQ ID NO: 93 below. In some embodiments, the blocking moiety comprises an amino acid sequence having at least about 80%, 85%, 90%, 95%, 96%, 97%, 98%, 99%, or 100% sequence identity to the sequence set forth in SEQ ID NO: 93. In some embodiments, the blocking moiety comprises an amino acid sequence having at least 95% sequence identity to the sequence set forth in SEQ ID NO: 93. In some embodiments, the blocking moiety comprises the sequence set forth in SEQ ID NO: 93. In some embodiments, the blocking moiety D3 domain of the IL-18 receptor alpha subunit comprises substitutions which remove glycosylation sites from the D3 domain. In some embodiments, the blocking moiety comprises the amino acid sequence set forth in SEQ ID NO: 94 below.

| SEQ | |
|---|---|
| ID | Blocking Moiety Sequence |
| 93 | SNIVPVLLGPKLNHVAVELGKNVRLNCSALLNEEDVIYW |
| | MFGEENGSDPNIHEEKEMRIMTPEGKWHASKVLRIENIGE |
| | SNLNVLYNCTVASTGGTDTKSFILVRKAD |
| 94 | SNIVPVLLGPKLNHVAVELGKNVRLDCSALLNEEDVIYW |
| | MFGEEDGSDPNIHEEKEMRIMTPEGKWHASKVLRIENIGE |
| | SNLNVLYDCTVASTGGTDTKSFILVRKAD |

In some embodiments, it may desirable to introduce one or more substitutions of amino acids to the sequence of the D3 domain of the IL-18 receptor alpha subunit when used as a blocking moiety in order to enhance or detune the binding of the D3 domain to the IL-18 polypeptide in the Act-IL-18 polypeptide.

The full length human IL-18 receptor alpha subunit has the sequence

(SEQ ID NO: 95)
MNCRELPLTLWVLISVSTAESCTSRPHITVVEGEPFYLKHCSCSLAHEIE
TTTKSWYKSSGSQEHVELNPRSSSRIALHDCVLEFWPVELNDTGSYFFQM
KNYTQKWKLNVIRRNKHSCFTERQVTSKIVEVKKFFQITCENSYYQTLVN
STSLYKNCKKLLLENNKNPTIKKNAEFEDQGYYSCVHFLHHNGKLFNITK
TFNITIVEDRSNIVPVLLGPKLNHVAVELGKNVRLNCSALLNEEDVIYWM
FGEENGSDPNIHEEKEMRIMTPEGKWHASKVLRIENIGESNLNVLYNCTV
ASTGGTDTKSFILVRKADMADIPGHVFTRGMIIAVLILVAVVCLVTVCVI
YRVDLVLFYRHLTRRDETLTDGKTYDAFVSYLKECRPENGEEHTFAVEIL
PRVLEKHFGYKLCIFERDVVPGGAVVDEIHSLIEKSRRLIIVLSKSYMSN
EVRYELESGLHEALVERKIKIILIEFTPVTDFTFLPQSLKLLKSHRVLKW
KADKSLSYNSRFWKNLLYLMPAKTVKPGRDEPEVLPVLSES
(UniProt ID: Q13478).

### Linking Peptides

In some embodiments, the artificial terminal moiety comprises one or more linking peptides. In some embodiments, the linking peptide of an artificial terminal moiety is positioned between the specific cleavage site and the IL-18 polypeptide (i.e., the linking peptide remains attached to the IL-18 polypeptide after cleavage) or is positions between the specific cleavage site and a blocking moiety, or both (e.g., the Act-IL-18 polypeptide has two linking peptides). In some embodiments, a linking peptide comprises from 1 to 50 amino acid, from 1 to 40 amino acids, from 1 to 30 amino acids, from 1 to 25 amino acids, from 1 to 20 amino acids, from 1 to 15 amino acids, from 1 to 10 amino acids, or from 1 to 5 amino acids. In some embodiments, the linking peptide comprises from 1 to 15 amino acids. In some embodiments, the linking peptide consists of amino acids glycine and serine. In some embodiments, the linking peptide consists of glycines. Non-limiting examples of a linking peptide include, but are not limited to (GS)ₙ (SEQ ID NO: 235), (GGS)ₙ (SEQ ID NO: 236), (GGGS)ₙ (SEQ ID NO: 237), (GGSG)ₙ (SEQ ID NO: 238), or (GGSGG)ₙ (SEQ ID NO: 239), (GGGGS)ₙ (SEQ ID NO: 240), wherein n is 1, 2, 3, 4, 5, 6, 7, 8, 9, or 10. For example, a linking peptide can be (GGGGS)₃ (SEQ ID NO: 241) or (GGGGS)₄ (SEQ ID NO: 242). Additional linking peptides can include GGGGSGGGGSGGGG (SEQ ID NO: 243). In embodiments where the artificial terminal moiety comprises multiple linking peptides, each linking peptide can be the same or different.

### Molecule Orientations

Act-IL-18 polypeptides provided herein can have a variety of orientations. In some embodiments, an Act-IL-18 polypeptide provided herein comprises an orientation according to any one of the below formulas:
(a) IL-18-CS;
(b) IL-18-LP₁—CS;
(c) IL-18-LP₁—CS-LP₂-BM;
(d) IL-18-CS-LP₁-BM;
(e) IL-18-CS-BM;
(f) IL-18-LP₁—CP-BM;
(g) BM-CS-IL-18;
(h) BM-LP₁—CS-IL-18;
(i) BM-LP₁—CS-LP₂-IL-18;
(j) BM-CS-LP₁-IL-18;
(k) CS-LP₁-IL-18; and
(l) CS-IL-18;
   wherein IL-18 is the IL-18 polypeptide, CS is a moiety comprising the specific cleavage site (e.g., a protease recognition sequence), LP₁ is a first linking peptide, LP₂ is a second linking peptide, and BM is a blocking moiety, and wherein the orientations are shown in an N-terminal to C-terminal order.

In some embodiments, the Act-IL-18 polypeptide comprises the orientation of formula (a). In some embodiments, the Act-IL-18 polypeptide comprises the orientation of formula (b). In some embodiments, the Act-IL-18 polypeptide comprises the orientation of formula (c). In some embodiments, the Act-IL-18 polypeptide comprises the orientation of formula (d). In some embodiments, the Act-IL-18 polypeptide comprises the orientation of formula (e). In some embodiments, the Act-IL-18 polypeptide comprises the orientation of formula (f). In some embodiments, the Act-IL-18 polypeptide comprises the orientation of formula (g). In some embodiments, the Act-IL-18 polypeptide comprises the orientation of formula (h). In some embodiments, the Act-IL-18 polypeptide comprises the orientation of formula (i). In some embodiments, the Act-IL-18 polypeptide comprises the orientation of formula (j). In some embodiments, the Act-IL-18 polypeptide comprises the orientation of formula (k). In some embodiments, the Act-IL-18 polypeptide comprises the orientation of formula (l).

### IL-18 Polypeptides

The Act-IL-18 polypeptides herein comprise IL-18 polypeptides. An IL-18 polypeptide of the instant disclosure can contain a number of modifications to WT IL-18 (SEQ ID NO: 1), including without limitation amino acid substitutions, deletions, additions, or attachment of polymer moieties.

In some embodiments, the IL-18 polypeptide comprises an amino acid sequence having at least about 80%, 85%, 90%, 95%, 96%, 97%, 98%, or 99% sequence identity to SEQ ID NO: 1. In some embodiments, the IL-18 polypeptide comprises an amino acid sequence having at least about 80%, 85%, 90%, 95%, 96%, 97%, 98%, 99%, or 100% identity to a sequence set forth in any one of SEQ ID NOs: 2-67. In some embodiments, the IL-18 polypeptide comprises an amino acid sequence having at least about 80%, 85%, 90%, 95%, 96%, 97%, 98%, 99%, or 100% identity to a sequence set forth in any one of SEQ ID NOs: 79-83. In some embodiments, the IL-18 polypeptide comprises the amino acid sequence set forth in SEQ ID NO: 30. In some embodiments, the IL-18 polypeptide comprises the amino acid sequence set forth in SEQ ID NO: 79. In some embodiments, the IL-18 polypeptide comprises the amino acid sequence set forth in SEQ ID NO: 80. In some embodiments, the IL-18 polypeptide comprises the amino acid sequence set forth in SEQ ID NO: 81. In some embodiments, the IL-18 polypeptide comprises the amino acid sequence set forth in SEQ ID NO: 82. In some embodiments, the IL-18 polypeptide comprises the amino acid sequence set forth in SEQ ID NO: 83.

### Amino Acid Substitutions of IL-18 Polypeptides

In some embodiments, the IL-18 polypeptide of an Act-IL-18 polypeptide described herein comprises a polypeptide of at least 1, at least 2, at least 3, at least 4, at least 5, at least 6, at least 7, at least 8 at least 9, or more substitutions at one or more amino acid residues, whereas the positions of the substitutions are relative to positions in IL-18 of SEQ ID NO:1. In some embodiments, the IL-18 polypeptide comprises 1 to 9 amino acid substitutions. In some embodiments, the Act-IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-18 polypeptide further comprises additional substitutions for a synthetic IL-18 polypeptide (e.g., homoserine substitutions, norleucine substitutions, O-methyl-homoserine substitutions, other unnatural or modified amino acids). It is expressly contemplated that these substitutions can be included in addition to the substitutions provided in this section (e.g., a synthetic IL-18 polypeptide can comprise the 1 to 9 amino acid substitutions discussed supra and additional synthetic IL-18 amino acid substitutions).

In some embodiments, the modified IL-18 polypeptide comprises at least one additional modification to the amino acid sequence of SEQ ID NO: 1 selected from: Y01X, F02X, E06X, S10X, V11X, D17X, C38X, M51X, K53X, D54X, S55X, T63X, C68X, E69X, K70X, C76X, E85X, M86X, T95X, D98X, AND C127X, wherein each X is independently a natural or non-natural amino acid. In some embodiments, the modified IL-18 polypeptide comprises at least one additional modification to the amino acid sequence of SEQ ID NO: 1 selected from: Y01G, F02A, E06K, S10T, V11I, D17N, C38S, C38A, C38Q, M51G, K53A, D54A, S55A, T63A, C68S, C68A, E69C, K70C, C76S, C76A, E85C, M86C, T95C, D98C, C127A, and C127S.

In one aspect, described herein is an Act-IL-18 polypeptide, comprising a modified IL-18 polypeptide comprising E06K and K53A, wherein residue position numbering of the modified IL-18 polypeptide is based on SEQ ID NO: 1 as a reference sequence. In some embodiments, the modified IL-18 polypeptide further comprises T63A. In some embodiments, the modified IL-18 polypeptide further comprises at least one of Y01X, S55X, F02X, D54X, C38X, C68X, E69X, K70X, C76X, or C127X, wherein each X is independently an amino acid or an amino acid derivative. In some embodiments, the modified IL-18 polypeptide further comprises at least one of Y01G, S55A, F02A, D54A, C38S, C38A, C68S, C68A, K70C, C76S, C76A, C127S, or C127A.

In some embodiments, the modified IL-18 peptide comprises at least one modification to the amino acid sequence of SEQ ID NO: 1, wherein the modification is E06X, K53X, S55X, or T63X, wherein X is a natural or non-natural amino acid. In some embodiments, the modified IL-18 peptide comprises at least two additional modifications to the amino acid sequence of SEQ ID NO: 1, wherein the modifications comprise E06X and K53X; E06X and S55X; K53X and S55X; E06X and T63X; or K53X and T63X, wherein X is a natural or non-natural amino acid. In some embodiments, the modified IL-18 peptide comprises at least three additional modifications to the amino acid sequence of SEQ ID NO: 1, wherein the modifications comprise E06X, K53X, and S55X; or E06X, K53X, and T63X, wherein X is a natural or non-natural amino acid. In some embodiments, the modified IL-18 peptide comprises at least four additional modifications to the amino acid sequence of SEQ ID NO: 1, wherein the modifications comprise E06X, K53X, S55X, and T63X; E06X, K53X, S55X, and Y01X; E06X, K53X, S55X, and F02X; E06X, K53X, S55X, and D54X; E06X, K53X, S55X, and M51X; or C38X, C68X, C76X, and C127X, wherein X is a natural or non-natural amino acid. In each embodiment wherein a plurality of amino acids residues are replaced with a natural or non-natural amino acid X, each X is independently the same or a different amino acid.

In some embodiments, the modified IL-18 peptide comprises at least one additional modification to the amino acid sequence of SEQ ID NO: 1, wherein the modification is E06K, V11I, K53A, S55A, or T63A. In some embodiments, the modified IL-18 peptide comprises at least two additional modifications to the amino acid sequence of SEQ ID NO: 1, wherein the modifications comprise E06K and K53A; E06K and S55A; K53A and S55A; E06K and T63A; or K53A and T63A. In some embodiments, the modified IL-18 peptide comprises at least three additional modifications to the amino acid sequence of SEQ ID NO: 1, wherein the modifications comprise E06K, K53A, and S55A; E06K, V 11, and K53A; E06K, C38A, and K53A; or E06K, K53A, and T63A. In some embodiments, the modified IL-18 peptide comprises at least four additional modifications to the amino acid sequence of SEQ ID NO: 1, wherein the modifications comprise E06K, K53A, S55A, and T63A; E06K, K53A, S55A, and Y01G; E06K, K53A, S55A, and F02A; E06K, K53A, S55A, and D54A; E06K, K53A, S55A, and M51G; or C38S, C68S, C76S, and C127S. In some embodiments, the modified IL-18 peptide comprises at least six modifications to the amino acid sequence of SEQ ID NO: 1, wherein the modifications comprise E06K, K53A, C38S, C68S, C76S, and C127S; or K53A, T63A, C38S, C68S, C76S, and C127S. In some embodiments, the modified IL-18 polypeptide comprises at least seven modifications to the sequence of SEQ ID NO: 1, wherein the seven modifications comprise E6K, VIII, C38A, K53A, T63A, C76A, C127A. In some embodiments, the modified IL-18 peptide comprises at least eight modifications to the amino acid sequence of SEQ ID NO: 1, wherein the modifications comprise Y01G, F02A, E06K, M51G, K53A, D54A, S55A, and T63A. In some embodiments, the modified IL-18 peptide comprises at least eight additional modifications to the amino acid sequence of SEQ ID NO: 1, wherein the modifications comprise Y01G, F02A, E06K, M51G, K53A, D54A, S55A, and T63A.

In one aspect, provided herein, is a modified IL-18 polypeptide with a polymer as provided herein (e.g., a polymer attached to a residue as provided herein), further comprising E06K and K53A, wherein residue position numbering of the modified IL-18 polypeptide is based on SEQ ID NO: 1 as a reference sequence. In some embodiments, the modified IL-18 polypeptide comprises an amino acid sequence at least 80%, at least 85%, at least 90%, at least 95%, or at least 98% identical to the amino acid sequence of SEQ ID NO: 30. In some embodiments, the modified IL-18 polypeptide comprises an amino acid sequence at least 80%, at least 85%, at least 90%, at least 95%, or at least 98% identical to the amino acid sequence of SEQ ID NO: 59. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue of SEQ ID NO: 1. In some embodiments, each cysteine residue is substituted with serine or alanine. In some embodiments, the modified IL-18 polypeptide comprises a polymer covalently attached to an amino acid residue. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue or a norleucine residue. In some embodiments, each methionine residue is substituted with an O-methyl-L-homoserine residue. In some embodiments, the modified IL-18 polypeptide comprises homoserine residues at positions 31, 116, and one of 63 and 75. In some embodiments, the modified IL-18 polypeptide comprises homoserine residues at positions 31, 116, 75, and one of 50, 57, 63, and 67. In some embodiments, the modified IL-18 polypeptide comprises homoserine residues at positions 31, 121, 75, and one of 50, 57, 63, and 67.

An IL-18 polypeptide of an Act-IL-18 polypeptide as described herein can comprise one or more non-canonical amino acids. “Non-canonical” amino acids can refer to amino acid residues in D- or L-form that are not among the 20 canonical amino acids generally incorporated into naturally occurring proteins. In some embodiments, one or more amino acids of the active form of the Act-IL-18 polypeptides are substituted with one or more non-canonical amino acids. Non-canonical amino acids include, but are not limited to N-alpha-(9-Fluorenylmethyloxycarbonyl)-L-azidolysine (Fmoc-L-Lys(N₃)—OH), N-alpha-(9-Fluorenylmethyloxycarbonyl)-L-biphenylalanine (Fmoc-L-Bip-OH), and N-alpha-(9-Fluorenylmethyloxycarbonyl)-O-benzyl-L-tyrosine (Fmoc-L-Tyr(Bzl)-OH.

Exemplary non-canonical amino acids include azido-lysine (AzK), hydroxylysine, allo-hydroxylysine, ε-N,N,N-trimethyllysine, ε-N-acetyllysine, ε-hydroxylysine, Fmoc-Lys (Me, Boc), Fmoc-Lys (Me)₃, Fmoc-Lys (palmitoyl), Fmoc-L-photo-lysine, DL-5-hydroxylysine, H-L-photo-lysine, and/or other similar amino acids. Example non-canonical amino acids also include D-methionine, selenocysteine, and/or other similar amino acids.

Exemplary non-canonical amino acids also include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl) alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, p-amino-L-phenylalanine, isopropyl-L-phenylalanine, an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a β-amino acid; a cyclic amino acid other than proline or histidine; an aromatic amino acid other than phenylalanine, tyrosine or tryptophan; or a combination thereof. In some embodiments, the non-canonical amino acids are selected from β-amino acids, homoamino acids, cyclic amino acids and amino acids with derivatized side chains. In some embodiments, the non-canonical amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, isodesmosine, allo-isoleucine, o-methylarginine, N^{α}-methylglycine, N^{α}-methylisoleucine, N^{α}-methylvaline, γ-carboxyglutamate, 0-phosphoserine, N^{α}-acetylserine, N^{α}-formylmethionine, 3-methylhistidine, and/or other similar amino acids.

In some embodiments, amino acid residues of the Act-IL-18 polypeptides are substituted with modified lysine residues. In some embodiments, the modified lysine residues comprise an amino, azide, allyl, ester, and/or amide functional groups. In some embodiments, the modified lysine residues contain conjugation handles which can serve as useful anchor points to attach additional moieties to the active form of the Act-IL-18 polypeptides. In some embodiments, the modified lysine residues have a structure built from precursors Structure 1, Structure 2, Structure 3, or Structure 4:

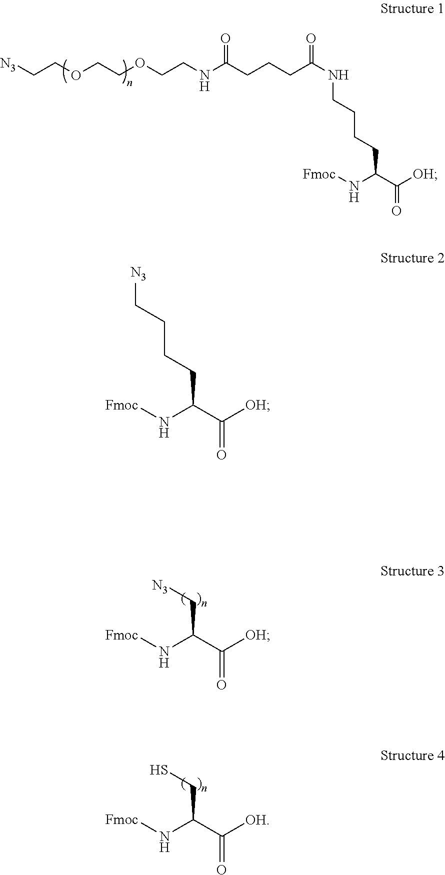

In some embodiments, the Act-IL-18 polypeptide contains a substitution for modified amino acid residues which can be used for attachment of additional functional groups which can be used to facilitate conjugation reaction or attachment of various payloads to the Act-IL-18 polypeptide (e.g., polymers). The substitution can be for a naturally occurring amino acid which is more amenable to attachment of additional functional groups (e.g., aspartic acid, cysteine, glutamic acid, lysine, serine, threonine, or tyrosine), a derivative of a modified version of any naturally occurring amino acid, or any unnatural amino acid (e.g., an amino acid containing a desired reactive group, such as a CLICK chemistry reagent such as an azide, alkyne, etc.). Non-limiting examples of such modified amino acid residues include the modified lysine, glutamic acid, aspartic acid, and cysteine provided below:

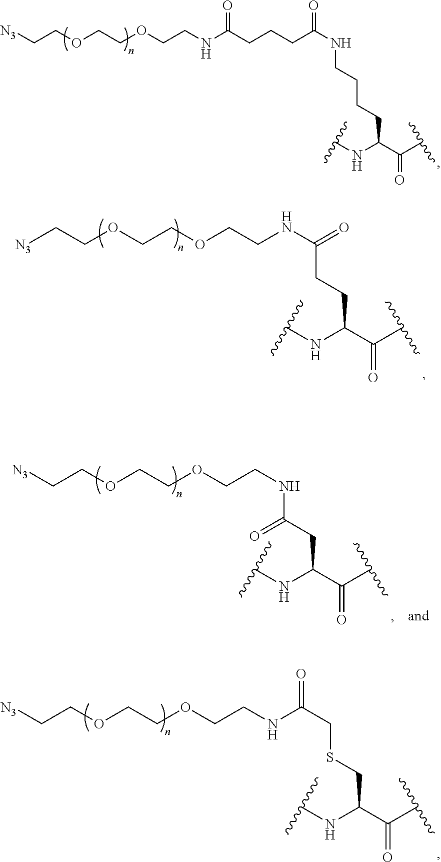

wherein each n is an integer from 1-30. These non-limiting examples of modified amino acid residues can be used at any location at which it is desirable to add an additional functionality (e.g., a polymer) to the Act-IL-18 polypeptide.

In some embodiments, any of structures 1-4, the modified lysine, the modified glutamic acid, the modified aspartic acid, or the modified cysteine provided above can be substituted for a different residue of the Act-IL-18 polypeptide (e.g., any of residues 68-70 or residues 80-100 using SEQ ID NO: 1 as a reference sequence) to allow for conjugation at a different site of the IL-18 polypeptide. The azide functionality may also be replaced with another suitable conjugation handle.

The conjugation handles provided herein can be any suitable reactive group capable of reacting with a complementary reactive group. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, potassium acyl trifluoroborate (KAT) ligations, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine, phosphine, trans-cyclooctene, and hydrazide. In some embodiments, the conjugation handle and complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, a group attached to the Act-IL-18 polypeptide (e.g., a polymer moiety or an additional polypeptide) comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the group attached to the IL-18 polypeptide (e.g., the polymer or the additional polypeptide) will comprise a reaction product of a conjugation handle with a complementary conjugation handle which was used to attach the group to the Act-IL-18 polypeptide.

### Polymers Attached to Act-IL-18 Polypeptides

In one aspect, described herein is a polypeptide that comprises a Act-IL-18 polypeptide, wherein the Act-IL-18 polypeptide comprises a covalently attached polymer. In some embodiments, a herein described Act-IL-18 polypeptide comprises one or more polymers covalently attached thereon. In some embodiments, the described Act-IL-18 polypeptide comprises 1, 2, 3, 4, 5, 6, 7, 8, 9, 10, or more polymers covalently attached to the Act-IL-18 polypeptide.

In some embodiments, the polymer comprises a conjugation handle, which can be used to further attach an additional moiety to the Act-IL-18 polypeptide (e.g., the addition of an additional polypeptide, such as an antibody). Any suitable reactive group capable of reacting with a complementary reactive group attached to another moiety can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, potassium acyl trifluoroborate (KAT) ligations, or a metal-mediated process such as olefin metathesis and Suzuki-Miyaura or Sonogashira cross-coupling.

In some embodiments, the IL-18 polypeptide comprises a polymer attached to a residue of the IL-18 polypeptide. In some embodiments, the polymer is attached to any one of residues 30-157 of the IL-18 polypeptide. In some embodiments, the polymer is covalently attached to a residue selected from residue 38, 68, 69, 70, 76, 78, 85, 86, 95, 98, 121, 127, and 144 of the IL-18 polypeptide. In some embodiments, the polymer is covalently attached to a residue selected from 68, 69, 70, 85, 86, 95, and 98 of the IL-18 polypeptide. In some embodiments, the polymer is covalently attached at residue 68 of the IL-18 polypeptide. In some embodiments, the polymer is covalently attached at residue 68 of the IL-18 polypeptide. In some embodiments, the polymer is covalently attached at residue 69 of the pro-IL-18 polypeptide. In some embodiments, the polymer is covalently attached at residue 70 of the IL-18 polypeptide. In some embodiments, the polymer is covalently attached at residue 85 of the IL-18 polypeptide. In some embodiments, the polymer is covalently attached at residue 86 of the IL-18 polypeptide. In some embodiments, the polymer is covalently attached at residue 98 of the IL-18 polypeptide.

In some embodiments, the polymer is covalently attached at residue 85. In some embodiments, the polymer is covalently attached at residue E85, E85C, E85D, E85Q, E85K, E85N, or E85Y. In some embodiments, the polymer is covalently attached at residue E85. In some embodiments, the polymer is covalently attached residue E85C. In some embodiments, the polymer is covalently attached to an unnatural amino acid at residue 85.

In some embodiments, the polymer is covalently attached at residue 86. In some embodiments, the polymer is covalently attached at residue M86C, M86D, M86Q, M86K, M86N, M86E, or M86Y. In some embodiments, the polymer is covalently attached M86C. In some embodiments, the polymer is covalently attached to an unnatural amino acid at residue 86.

In some embodiments, the polymer is covalently attached at residue 95. In some embodiments, the polymer is covalently attached at residue T95, T95C, T95D, T95Q, T95K, T95N, T95E, or T95Y. In some embodiments, the polymer is covalently attached at residue T95C, T95D, T95Q, T95K, T95N, T95E, or T95Y. In some embodiments, the polymer is covalently attached at residue T95C. In some embodiments, the polymer is covalently attached to an unnatural amino acid at residue 95.

In some embodiments, the polymer is covalently attached at residue 98. In some embodiments, the polymer is covalently attached at residue D98, D98C, D98Q, D98K, D98N, D98E, or D98Y. In some embodiments, the polymer is covalently attached at residue D98C. In some embodiments, the polymer is covalently attached to an unnatural amino acid at residue 98.

In one aspect, provided herein, is a Act-IL-18 polypeptide with a polymer as provided herein (e.g., a polymer attached to a residue as provided herein), further comprising E06K and K53A, wherein residue position numbering of the Act-IL-18 polypeptide is based on SEQ ID NO: 1 as a reference sequence. In some embodiments, the Act-IL-18 polypeptide comprises an amino acid sequence at least 80%, at least 85%, at least 90%, at least 95%, or at least 98% identical to the amino acid sequence of SEQ ID NO: 30. In some embodiments, the Act-IL-18 polypeptide comprises the amino acid sequence of SEQ ID NO: 30. In some embodiments, the Act-IL-18 polypeptide comprises an amino acid sequence at least 80%, at least 85%, at least 90%, at least 95%, or at least 98% identical to the amino acid sequence of SEQ ID NO: 59. In some embodiments, the Act-IL-18 polypeptide comprises the amino acid sequence of SEQ ID NO: 59. In some embodiments, the Act-IL-18 polypeptide further comprises an amino acid substitution at one or more cysteine residues. In some embodiments, the Act-IL-18 polypeptide comprises one or more cysteines substituted with either serine or alanine. In some embodiments, the Act-IL-18 polypeptide comprise amino acid substitutions at each cysteine residue of SEQ ID NO: 1. In some embodiments, each cysteine residue is substituted with serine or alanine. In some embodiments, the Act-IL-18 polypeptide comprises a polymer covalently attached to an amino acid residue.

The Active form of the IL-18 Polypeptide

In some embodiments, cleavage of the specific cleavage site of the artificial terminal moiety converts the Act-IL-18 into an active form of the IL-18 polypeptide. As used herein, the active form of the IL-18 polypeptide refers to the cleaved version of the IL-18 polypeptide which possesses some or all of the activity associated with the IL-18 polypeptide which is inactivated by the artificial terminal moiety. Additionally, unless context clearly indicates otherwise, reference to simply “the IL-18 polypeptide” refers to an IL-18 polypeptide which was never prepared in an activatable form (E.g., it refers to the base IL-18 polypeptide on which an Act-IL-18 polypeptide is based). In some embodiments, the active form of the IL-18 polypeptide comprises a portion the artificial terminal moiety still attached to the IL-18 polypeptide (e.g., a subset of amino acid residues of the artificial terminal moiety). In some embodiments, the active form of the IL-18 polypeptide is the same as the IL-18 polypeptide (e.g., has the same amino acid sequence as the IL-18 polypeptide because the entire artificial terminal moiety has been cleaved).

In some embodiments, the active form of the IL-18 polypeptide provided herein display reduced binding to the IL-18 binding protein (IL-18BP) relative to WT-IL-18. The active form of the IL-18 polypeptides may also display binding characteristics for the IL-18Rαβ that differ from wild-type IL-18 (e.g., a higher affinity for the IL-18Rαβ heterodimer or a lower affinity for the IL-18Rαβ heterodimer). In preferred embodiments, the affinity for IL-18Rαβ of the active form of the IL-18 polypeptide is not substantially lower than the affinity of WT IL-18 for IL-18Rαβ(e.g., the active form of the Act-IL-18 polypeptide's affinity for IL-18Rαβ is no less than about 500× lower than wild type IL-18).

In some embodiments, the active form of the IL-18 polypeptides display increased affinity for an IL-18 receptor alpha subunit (IL-18Rα) or an IL-18 receptor beta subunit (IL-18Rβ) relative to wild type IL-18. In some embodiments, the active form of the IL-18 polypeptides have an increased affinity for the IL-18Rα/β heterodimer relative to IL-18 WT. In one aspect, the active form of the IL-18 polypeptides described herein have decreased affinity for the IL-18Rα/β heterodimer relative to wild type IL-18.

In some embodiments, the binding affinity between the active form of the IL-18 polypeptides and IL-18Rα is the same as or lower than the binding affinity between a wild-type IL-18 and IL-18Rα. In some embodiments, the binding affinity between the active form of the IL-18 polypeptides and IL-18Rα is the same as or higher than the binding affinity between a wild-type IL-18 and IL-18Rα. In some embodiments, the binding affinity between the active form of the IL-18 polypeptides and IL-18Rβ is the same as or lower than the binding affinity between a wild-type IL-18 and IL-18Rβ. In some embodiments, the binding affinity between the active form of the IL-18 polypeptides and IL-18Rβ is the same as or higher than the binding affinity between a wild-type IL-18 and IL-18Rβ. In some embodiments, the binding affinity between the active form of the IL-18 polypeptides and the IL-18Rα/β heterodimer is the same as or lower than the binding affinity between a wild-type IL-18 and the IL-18Rα/β heterodimer. In some embodiments, the binding affinity between the active form of the IL-18 polypeptides and the IL-18Rα/β heterodimer is the same as or higher than the binding affinity between a wild-type IL-18 and the IL-18Rα/β heterodimer.

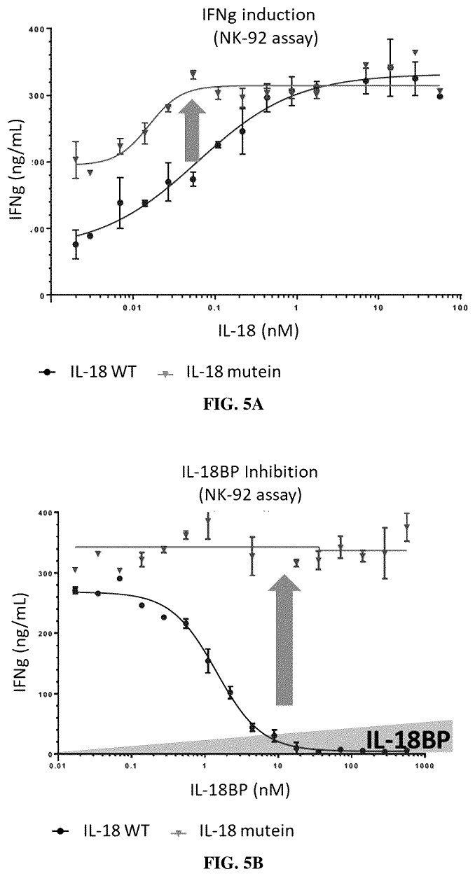

In some embodiments, an active form of the IL-18 polypeptide provided herein displays an ability to induce interferon gamma (IFNγ) production after administration to a subject. In some embodiments, the ability to induce IFNγ is comparable to that of a wild type IL-18 (e.g., displays an EC50 for IFNγ induction that is within about 10-fold of that of a wild type IL-18). An exemplary IL-18 polypeptide displaying this characteristic is shown in FIG. 5A, which shows a comparison of IFNγ production (ng/mL, y-axis) as a function of concentration of a wild type versus an IL-18 polypeptide (mutein) (nM, x-axis).

In some embodiments, an active form of the Act-IL-18 polypeptide provided herein also display a reduced binding IL-18 binding protein (IL-18BP). In some embodiments, the active form of the IL-18 polypeptide provided herein can induce IFNγ even in the presence of IL-18BP (e.g., the ability of the active form of the Act-IL-18 polypeptide to induce IFNγ is not substantially inhibited by the presence of IL-18BP) (nM, x-axis). An example of an IL-18 polypeptide with this property compared to wild type IL-18 is shown in FIG. 5B, which shows IFNγ production (ng/mL, y-axis) as a function of IL-18BP concentration (nM, x-axis) in a sample treated with the same level of wild type IL-18 (circles) or the IL-18 polypeptide (inverted triangles). In some embodiments, an active form of IL-18 polypeptide provided herein displays a similar or only slightly reduced ability to induce IFNγ production compared to wild type IL-18.

In some embodiments, an active form of IL-18 polypeptide provided herein displays a significant reduction in inhibition of the ability to induce IFNγ production in the presence of IL-18BP compared to wild type IL-18. In some embodiments, an active form of IL-18 polypeptide provided herein displays a similar or only slightly reduced ability to induce IFNγ production compared to wild type IL-18, and a significant reduction in inhibition of the ability to induce IFNγ production in the presence of IL-18BP compared to wild type IL-18.

### Biological Activity of Act-IL-18 and the Active Form of the IL-18 Polypeptide

### Binding Affinity

In some embodiments the Act-IL-18 exhibits a decreased affinity for the IL-18 receptor of at least 10-fold, at least 100 fold, at least 500 fold, at least 1000 fold lower in comparison to wild type IL-18 or the active form of the IL-18 polypeptide. In some embodiments, the Act-IL-18 exhibits an increased EC₅₀ for the production of IFN-γ that is at least 5 fold, at least 10-fold, at least 100 fold, at least 500 fold, at least 1000 fold higher in comparison to wild type IL-18 or the active from of the IL-18 polypeptide. In some embodiments, the Act-IL-18 exhibits an increased EC₅₀ for the production of IFN-γ that is at least 5 fold, at least 10-fold, at least 100 fold, at least 500 fold, at least 1000 fold higher in comparison to the active form of the IL-18 polypeptide. In some embodiments, the Act-IL-18 exhibits an increased EC₅₀ for the production of IFN-γ that is at least 5 fold, at least 10-fold, at least 100 fold, at least 500 fold, at least 1000 fold higher in comparison to the IL-18 polypeptide.

In some embodiments, the active form of the IL-18 polypeptide provided herein exhibits reduced affinity for IL-18 binding protein (IL-18BP) compared to WT IL-18 (SEQ ID NO: 1). In some embodiments, the active form of the IL-18 polypeptide exhibits at least 2-fold, at least 5-fold, at least 10-fold, at least 20-fold, at least 30-fold, at least 40-fold, at least 50-fold, at least 60-fold, at least 70-fold, at least 80-fold, at least 90-fold, or at least 100-fold lower affinity for IL-18BP compared to WT IL-18. In some embodiments, the active form of the IL-18 polypeptide exhibits at least a 10-fold lower affinity for IL-18BP compared to WT IL-18. In some embodiments, the active form of the IL-18 polypeptide exhibits at least a 20-fold lower affinity for IL-18BP compared to WT IL-18. In some embodiments, the active form of the IL-18 polypeptide exhibits at least a 50-fold lower affinity for IL-18BP compared to WT IL-18. In some embodiments, the active form of the IL-18 polypeptide exhibits at least an 80-fold lower affinity for IL-18BP compared to WT IL-18. In some embodiments, the active form of the IL-18 polypeptide exhibits at least a 100-fold lower affinity for IL-18BP compared to WT IL-18.

In some embodiments, the active form of the IL-18 polypeptide provided herein exhibits a reduced binding to IL-18BP as measured by K_{D}. In some embodiments, the active form of the IL-18 polypeptide exhibits a K_{D} with IL-18BP of at least about 1 nM, at least about 5 nM, at least about 10 nM, at least about 15 nM, at least about 20 nM, at least about 25 nM, at least about 50 nM, at least about 100 nM, at least about 200 nM, at least about 300 nM, at least about 400 nM, or at least about 500 nM. In some embodiments, the active form of the IL-18 polypeptide exhibits a K_{D} with IL-18BP of at least about 1 nM. In some embodiments, the active form of the IL-18 polypeptide exhibits a K_{D} with IL-18BP of at least about 5 nM. In some embodiments, the active form of the IL-18 polypeptide exhibits a K_{D} with IL-18BP of at least about 50 nM. In some embodiments, the active form of the IL-18 polypeptide exhibits a K_{D} with IL-18BP of at least about 100 nM. In some embodiments, the active form of the IL-18 polypeptide exhibits a K_{D} with IL-18BP of at least about 500 nM.

In some embodiments, the active form of the IL-18 polypeptide displays at most an only slightly diminished affinity for IL-18Rαβ compared to WT IL-18 (SEQ ID NO: 1). In some embodiments, the active form of the IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18 Rαμ as compared to the affinity of WT IL-18 for IL-18Rαβ. In some embodiments, the active form of the IL-18 polypeptide exhibits at most a 10-fold lower affinity for IL-18Rαβ as compared to the affinity of WT IL-18 for IL-18Rαβ. In some embodiments, the active form of the IL-18 polypeptide exhibits at most a 20-fold lower affinity for IL-18Rαβ as compared to the affinity of WT IL-18 for IL-18Rαμ. In some embodiments, the active form of the IL-18 polypeptide exhibits at most a 50-fold lower affinity for IL-18Rαβ as compared to the affinity of WT IL-18 for IL-18Rαβ. In some embodiments, the active form of the IL-18 polypeptide exhibits at most a 100-fold lower affinity for IL-18 Rαμ as compared to the affinity of WT IL-18 for IL-18Rαβ. In some embodiments, the active form of the IL-18 polypeptide exhibits an increased affinity for IL-18Rαβ compared to WT IL-18. In some embodiments, the affinity is increased by at least 2-fold, at least 4-fold, at least 6-fold, at least 8-fold, or at least 10-fold compared to WT IL-18.

In some embodiments, the active form of the IL-18 polypeptide displays at most an only slightly diminished affinity for IL-18Rαβ compared to the corresponding IL-18 polypeptide without the artificial terminal moiety. In some embodiments, the active form of the IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18 Rαμ as compared to the affinity of the corresponding IL-18 polypeptide without the artificial terminal moiety for IL-18Rαβ. In some embodiments, the active form of the IL-18 polypeptide exhibits at most a 2-fold lower affinity for IL-18Rαβ as compared to the affinity of the corresponding IL-18 polypeptide without the artificial terminal moiety for IL-18Rαμ. In some embodiments, the active form of the IL-18 polypeptide exhibits at most a 3-fold lower affinity for IL-18Rαβ as compared to the affinity of the corresponding IL-18 polypeptide without the artificial terminal moiety for IL-18Rαβ. In some embodiments, the active form of the IL-18 polypeptide exhibits at most a 4-fold lower affinity for IL-18Rαβ as compared to the affinity of the corresponding wild type IL-18 polypeptide without the N-terminal domain for IL-18Rαβ. In some embodiments, the active form of the IL-18 polypeptide exhibits at most a 5-fold lower affinity for IL-18 Rαμ as compared to the affinity of the corresponding IL-18 polypeptide without the artificial terminal moiety for IL-18Rαβ. In some embodiments, the active form of the IL-18 polypeptide exhibits at most a 10-fold lower affinity for IL-18Rαβ as compared to the affinity of the corresponding IL-18 polypeptide without the artificial terminal moiety for IL-18Rαβ.

In some embodiments, the active form of the IL-18 polypeptide provided herein exhibits at most only a slight reduction in binding to IL-18Rαβ as measured by K_{D}. In some embodiments, the active form of the IL-18 polypeptide exhibits a K_{D} with IL-18Rαβ of at most about 10 nM, at most about 20 nM, at most about 30 nM, at most about 50 nM, at most about 75 nM, at most about 100 nM, or at most about 200 nM. In some embodiments, the active form of the IL-18 polypeptide exhibits a K_{D} with IL-18Rαβ of at most about 20 nM. In some embodiments, the active form of the Act-IL-18 polypeptide exhibits a K_{D} with IL-18Rαβ of at most about 30 nM. In some embodiments, the active form of the IL-18 polypeptide exhibits a K_{D} with IL-18Rαβ of at most about 40 nM. In some embodiments, the active form of the IL-18 polypeptide exhibits a K_{D} with IL-18Rαβ of at most about 50 nM. In some embodiments, the active form of the IL-18 polypeptide exhibits an increase in binding to IL-18Rαβ compared to WT IL-18 as measured by K_{D}. In some embodiments, the active form of the IL-18 polypeptide has a K_{D} with IL-18Rαβ of at most about 2 nM, at most about 1 nM, at most about 0.5 nM, or at most about 0.2 nM.

In some embodiments, the active form of the IL-18 polypeptide exhibits a wide window in which the active form of the IL-18 polypeptide will bind to IL-18Rαβ even in the presence of IL-18BP. In some embodiments, this window can be measured by a ratio of K_{D} of the Act-IL-18/IL-18BP interaction over K_{D} of the IL-18/IL-18Rαβ interaction, where a larger number indicates a larger window in which the active form of the Act-IL-18 polypeptide is expected to be active in vivo. In some embodiments, the active form of the IL-18 polypeptide exhibits a ratio of K_{D} of the IL-18/IL-18BP interaction over K_{D} of the IL-18/IL-18Rαβ interaction of at least about 1, at least about 2, at least about 3, at least about 4, at least about 5, at least about 10, at least about 15, at least about 20, at least about 25, at least about 30, at least about 35, at least about 40, at least about 45, or at least about 50. In some embodiments, the active form of the IL-18 polypeptide exhibits a ratio of K_{D} of the IL-18/IL-18BP interaction over K_{D} of the IL-18/IL-18Rαβ interaction of at least about 2. In some embodiments, the active form of the IL-18 polypeptide exhibits a ratio of K_{D} of the IL-18/IL-18BP interaction over K_{D} of the IL-18/IL-18Rαβ interaction of at least about 5. In some embodiments, the active form of the IL-18 polypeptide exhibits a ratio of K_{D} of the IL-18/IL-18BP interaction over K_{D} of the IL-18/IL-18Rαβ interaction of at least about 10. In some embodiments, the active form of the IL-18 polypeptide exhibits a ratio of K_{D} of the IL-18/IL-18BP interaction over K_{D} of the IL-18/IL-18Rαβ interaction of at least about 25. In some embodiments, the active form of the IL-18 polypeptide exhibits a ratio of K_{D} of the IL-18/IL-18BP interaction over K_{D} of the IL-18/IL-18Rαβ interaction of at least about 30. In some embodiments, the active form of the IL-18 polypeptide exhibits a ratio of K_{D} of the IL-18/IL-18BP interaction over K_{D} of the IL-18/IL-18Rαβ interaction of at least about 40. In some embodiments, the active form of the IL-18 polypeptide exhibits a ratio of K_{D} of the IL-18/IL-18BP interaction over K_{D} of the IL-18/IL-18Rαμ interaction of at least about 45. In some embodiments, the active form of the IL-18 polypeptide exhibits a ratio of K_{D} of the IL-18/IL-18BP interaction over K_{D} of the IL-18/IL-18Rαβ interaction of at least about 50.

### Functional Activity of the Activated Form of the IL-18 Polypeptide

In some embodiments, the active form of the IL-18 polypeptides provided herein display one or more activities associated with WT IL-18. In some embodiments, the active form of the IL-18 polypeptide exhibits a similar ability to signal through the IL-18 receptor (IL-18Rαβ) but lacks the ability or displays a reduced ability to be inhibited by IL-18BP. In some embodiments, the active form of the IL-18 polypeptide's ability to signal through IL-18Rαβ is reduced compared to WT IL-18 by only a small amount.

In some embodiments, the active form of the IL-18 polypeptide modulates IFNγ production when in contact with a cell (e.g., an immune cell, such as an NK cell). In some embodiments, the active form of the IL-18 polypeptide's ability to modulate IFNγ production is measured as a half-maximal effective concentration (EC₅₀). In some embodiments, an EC₅₀ (nM) of the active form of the Act-IL-18 polypeptide's ability to induce IFNγ is less than 10-fold higher than, less than 5-fold higher than, or less than an EC₅₀ (nM) of an IL-18 polypeptide of SEQ ID NO: 1. In some embodiments, the EC₅₀ of the active form of the Act-IL-18 polypeptide's ability to induce IFNγ is less than 10-fold higher than an EC₅₀ (nM) of an IL-18 polypeptide of SEQ ID NO: 1. In some embodiments, the EC₅₀ of the active form of the Act-IL-18 polypeptide's ability to induce IFNγ is less than 5-fold higher than an EC₅₀ (nM) of an IL-18 polypeptide of SEQ ID NO: 1. In some embodiments, the EC₅₀ of the active form of the IL-18 polypeptide's ability to induce IFNγ is less than an EC₅₀ (nM) of an IL-18 polypeptide of SEQ ID NO: 1. In some embodiments, the EC₅₀ of the active form of the IL-18 polypeptide's ability to induce IFNγ is less than 10-fold higher than, less than 8-fold higher than, less than 6-fold higher than, less than 5-fold higher than, less than 4-fold higher than, less than 3-fold higher than, or less than 2-fold higher than an EC₅₀ (nM) of an IL-18 polypeptide of SEQ ID NO: 1. In some embodiments, the EC₅₀ of the active form of the IL-18 polypeptide's ability to induce IFNγ is measured by an IFNγ induction cellular assay.

In some embodiments, an EC₅₀ of the active form of the IL-18 polypeptide's ability to induce IFNγ production is less than about 100 nM, less than about 75 nM, less than about 50 nM, less than about 40 nM, less than about 30 nM, less than about 20 nM, less than about 15 nM, or less than about 10 nM. In some embodiments, an EC₅₀ of the active form of the IL-18 polypeptide's ability to induce IFNγ production is less than about 100 nM. In some embodiments, an EC₅₀ of the active form of the IL-18 polypeptide's ability to induce IFNγ production is less than about 50 nM. In some embodiments, an EC₅₀ of the active form of the IL-18 polypeptide's ability to induce IFNγ production is less than about 10 nM.

In some embodiments, the active form of the IL-18 exhibits a reduced ability to have its IFNγ induction activity inhibited by IL-18BP compared to WT IL-18. In some embodiments, the active form of the IL-18 displays a half-maximal inhibitory concentration (IC₅₀) by IL-18BP which is at least about 10-fold higher than, at least about 20-fold higher than, at least about 50-fold higher than, at least about 75-fold higher than, at least about 100-fold higher than, at least about 200-fold higher than, at least about 300-fold higher than, at least about 400-fold higher than, at least about 500-fold higher than, at least about 600-fold higher than, at least about 700-fold higher than, at least about 800-fold higher than, at least about 900-fold higher than, or at least about 1000-fold higher than an IC₅₀ of WT IL-18's inhibition by IL-18BP. In some embodiments, the active form of the IL-18 displays a half-maximal inhibitory concentration (IC₅₀) by IL-18BP which is at least about 100-fold higher than an IC₅₀ of WT IL-18's inhibition by IL-18BP. In some embodiments, the active form of the IL-18 displays a half-maximal inhibitory concentration (IC₅₀) by IL-18BP which is at least about 500-fold higher than an IC₅₀ of WT IL-18's inhibition by IL-18BP. In some embodiments, the active form of the IL-18 displays a half-maximal inhibitory concentration (IC₅₀) by IL-18BP which is at least about 1000-fold higher than an IC₅₀ of WT IL-18's inhibition by IL-18BP.

In some embodiments, the active form of the IL-18 polypeptide exhibits a favorable ratio of half-maximal inhibitory concentration (IC₅₀) by IL-18BP over a half-maximal effective concentration (EC₅₀) of IFNγ induction (IC₅₀/EC₅₀ ratio). In some embodiments, the IC₅₀/EC₅₀ ratio is increased compared to WT IL-18. In some embodiments, the IC₅₀/EC₅₀ ratio is increased by at least about 2-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, or at least about 1000-fold compared to WT IL-18. In some embodiments, the IC₅₀/EC₅₀ ratio is increased by at least about 10-fold compared to WT IL-18. In some embodiments, the IC₅₀/EC₅₀ ratio is increased by at least about 100-fold compared to WT IL-18. In some embodiments, the IC₅₀/EC₅₀ ratio is increased by at least about 500-fold compared to WT IL-18. In some embodiments, the IC₅₀/EC₅₀ ratio of the active form of the Act-IL-18 polypeptide is at least about 2, at least about 5, at least about 10, at least about 50, at least about 100, at least about 250, or at least about 500.

In some embodiments, the active form of the IL-18 polypeptide modulates IFNγ production, and wherein an EC₅₀ (nM) of the active form of the Act-IL-18 polypeptide against IFNγ is less than an EC₅₀ (nM) of an IL-18 polypeptide of SEQ ID NO: 1. In some embodiments, the EC₅₀ (nM) of the active form of the Act-IL-18 polypeptide against IFNγ is at least 10-fold less than the EC₅₀ (nM) of an IL-18 polypeptide of SEQ ID NO: 1. In some embodiments, the EC₅₀ (nM) of the active form of the Act-IL-18 polypeptide against IFNγ is about 10-fold less than the EC₅₀ (nM) of an IL-18 polypeptide of SEQ ID NO: 1. In some embodiments, the EC₅₀ (nM) of the active form of the Act-IL-18 polypeptide against IFNγ is about 15-fold less than the EC₅₀ (nM) of a n IL-18 polypeptide of SEQ ID NO: 1.

Relative Activity of the Activatable and Active form of IL-18

In some embodiments, the activated form of the IL-18 polypeptide (e.g., after cleavage of the specific cleavage site) exhibits an enhanced activity associated with IL-18 compared to the Act-IL-18 polypeptide with the specific cleavage site intact. In some embodiments, the active form of the IL-18 polypeptide exhibits an activity which is enhanced by at least 5-fold, 10-fold, 20-fold, 50-fold, 100-fold, 200-fold, 500-fold, 1000-fold, 2000-fold, 5000-fold, 10000-fold, 15000-fold, or 20,000-fold higher than the Act-IL-18 polypeptide. Such activities can include induction of production of IFNγ in a cell (e.g., an immune cell such as an NK cell), activation of signaling through the IL-18 receptor (e.g., in a reporter assay), or another in vitro or in vivo activity. In some embodiments, the activated form of the IL-18 polypeptide exhibits enhanced binding to the IL-18 receptor or a subunit thereof (e.g., the IL-18 receptor alpha subunit) compared to the Act-IL-18 polypeptide (e.g., has a K_{D} which is at least 10-fold, 20-fold, 50-fold, or 100-fold lower).

In some embodiments, the Act IL-18 polypeptide exhibits a half-maximal effective concentration (EC₅₀) for IL-18 receptor signaling activity (e.g., in a HEK-Blue reporter assay) which is higher than that of the activated form of the IL-18 polypeptide. In some embodiments, the Act IL-18 polypeptide exhibits an EC₅₀ for IL-18 receptor signaling activity which is at least 1,000-fold higher, 2,000-fold higher, 5,000-fold higher, 10,000-fold higher, 15,000-fold-higher, or 20,000-fold higher than the activated form of the IL-18 polypeptide. In some embodiments, the Act IL-18 polypeptide exhibits an EC₅₀ for IL-18 receptor signaling activity which is at least 1,000-fold higher than the activated form of the IL-18 polypeptide. In some embodiments, the Act IL-18 polypeptide exhibits an EC₅₀ for IL-18 receptor signaling activity which is at least 5,000-fold higher than the activated form of the IL-18 polypeptide. In some embodiments, the Act IL-18 polypeptide exhibits an EC₅₀ for IL-18 receptor signaling activity which is at least 10,000-fold higher than the activated form of the IL-18 polypeptide. In some embodiments, the Act IL-18 polypeptide exhibits an EC₅₀ for IL-18 receptor signaling activity which is at least 20,000-fold higher than the activated form of the IL-18 polypeptide.

In some embodiments, the Act-IL-18 polypeptide exhibits only a modest reduction in activity compared to the activated form of the IL-18 polypeptide. In some embodiments, the Act IL-18 polypeptide exhibits a half-maximal effective concentration (EC₅₀) for IL-18 receptor signaling activity which is from about 10-fold higher to about 100-fold higher than the activated form of the IL-18 polypeptide. In some embodiments, the Act IL-18 polypeptide exhibits an EC₅₀ for IL-18 receptor signaling activity which is from about 10-fold higher to about 50-fold higher than the activated form of the IL-18 polypeptide.

In some embodiments, the activated form of the IL-18 polypeptide has a comparable activity compared that of the IL-18 polypeptide from which the Act-IL-18 polypeptide is derived. In some embodiments, the activated form of the IL-18 polypeptide exhibits a half-maximal effective concentration (EC₅₀) for IL-18 receptor signaling activity which is within about 10-fold of the IL-18 polypeptide.

### III. Pharmaceutical Compositions

In one aspect, described herein is a pharmaceutical composition comprising: an Act-IL-18 polypeptide described herein; and a pharmaceutically acceptable carrier or excipient. In some embodiments, the pharmaceutical composition comprises a plurality of the Act-IL-18 polypeptides. In some embodiments, the pharmaceutical compositions further comprises one or more excipient selected from a carbohydrate, an inorganic salt, an antioxidant, a surfactant, or a buffer.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

In some embodiments, the pharmaceutical composition comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

In certain embodiments, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

In certain embodiments, the pharmaceutical composition comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

In certain embodiments, the pharmaceutical composition comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, Tris, HEPES, CHAPS, or combinations thereof.

In some embodiments, the pharmaceutical composition is formulated for parenteral or enteral administration. In some embodiments, the pharmaceutical composition is formulated for intravenous or subcutaneous administration. In some embodiments, the pharmaceutical composition is in a lyophilized form.

In one aspect, described herein is a liquid or lyophilized composition that comprises a described Act-IL-18 polypeptide. In some embodiments, the Act-IL-18 polypeptide is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising phosphate buffered saline solution (pH 7.4) with 50 mg/mL mannitol. In some embodiments, the pharmaceutical composition is a lyophilized composition which is reconstituted shortly before administration to a subject.

The Act-IL-18 polypeptides described herein can be in a variety of dosage forms. In some embodiments, the Act-IL-18 polypeptide is dosed as a lyophilized powder. In some embodiments, the Act-IL-18 polypeptide is dosed as a suspension. In some embodiments, the Act-IL-18 polypeptide is dosed as a solution. In some embodiments, the Act-IL-18 polypeptide is dosed as an injectable solution. In some embodiments, the Act-IL-18 polypeptide is dosed as an IV solution.

### IV. Host Cells

In one aspect, described herein is a host cell expressing the Act-IL-18 polypeptide.

In one aspect, described herein is a method of producing the Act-IL-18 polypeptide, wherein the method comprises expressing the Act-IL-18 polypeptide in a host cell.

In some embodiments, the host cell is a prokaryotic cell or a eukaryotic cell. In some embodiments, the host cell is a mammalian cell, an avian cell, or an insect cell. In some embodiments, the host cell is a mammalian cell, an avian cell, a fungal cell, or an insect cell. In some embodiments, the host cell is a CHO cell, a COS cell, or a yeast cell.

### V. Method of Treatment

In one aspect, described herein, is a method of treating cancer in a subject in need thereof, comprising: administering to the subject an effective amount of an Act-IL-18 polypeptide or a pharmaceutical composition as described herein.

In another aspect, described herein, is an Act-IL-18 polypeptide provided herein for use in treatment of cancer in a subject in need thereof. In another aspect, described herein, is a Act-IL-18 polypeptide provided herein for in the manufacture of a medicament for treatment of cancer in a subject in need thereof.

In some embodiments, the cancer is a solid cancer. In some embodiments, wherein the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

In some embodiments, the Act-IL-18 polypeptide is administered in a single dose of the effective amount of the Act-IL-18 polypeptide, including further embodiments in which (i) the Act-IL-18 polypeptide is administered once a day; or (ii) the Act-IL-18 polypeptide is administered to the subject multiple times over the span of one day. In some embodiments, the Act-IL-18 polypeptide is administered daily, every other day, 3 times a week, once a week, twice a week, every 2 weeks, every 3 weeks, every 4 weeks, every 5 weeks, every 6 weeks, every 12 weeks, every 3 days, every 4 days, every 5 days, every 6 days, 2 times a week, 3 times a week, 4 times a week, 5 times a week, 6 times a week, once a month, twice a month, 3 times a month, 4 times a month, once every 2 months, once every 3 months, once every 4 months, once every 5 months, or once every 6 months.

In some embodiments, the method further comprises reconstituting a lyophilized form of the Act-IL-18 polypeptide or the pharmaceutical composition. In some embodiments, the Act-IL-18 polypeptide or the pharmaceutical composition is reconstituted before administration. In some embodiments, the composition is reconstituted immediately before administration, up to about 5 minutes before administration, up to about 20 minutes before administration, up to about 40 minutes before administration, up to an hour before administration, or up to about four hours before administration.

### Exemplary IL-18 Sequences

The sequences provided in the table below represent exemplary IL-18 polypeptide which can be part of Act-IL-18 polypeptides as provided herein. In some embodiments, the IL-18 polypeptide of the Act-IL-18 polypeptide comprises an amino acid sequence having at least 80%, 85%, 90%, 95%, 96%, 97%, 98%, 99%, or 100% sequence identity to a sequence set forth in any one of SEQ ID Nos: 1-67. In some embodiments, the IL-18 polypeptide of the Act-IL-18 polypeptide comprises an amino acid sequence having at least 80%, 85%, 90%, 95%, 96%, 97%, 98%, 99%, or 100% sequence identity to a sequence set forth in any one of SEQ ID Nos: 79-83. In some embodiments, the IL-18 polypeptide of the Act-IL-18 polypeptide comprises an amino acid sequence as set forth in any one of SEQ ID Nos: 1-67. In some embodiments, the Act-IL-18 polypeptide comprises the amino acid sequence set forth in SEQ ID NO: 30. In some embodiments, the IL-18 polypeptide of the Act-IL-18 polypeptide comprises an amino acid sequence as set forth in any one of SEQ ID Nos: 79-83. In some embodiments, the IL-18 polypeptide of the Act-IL-18 polypeptide comprises the amino acid sequence set forth in SEQ ID NO: 79. In some embodiments, the IL-18 polypeptide of the Act-IL-18 polypeptide comprises the amino acid sequence set forth in SEQ ID NO: 80. In some embodiments, the IL-18 polypeptide of the Act-IL-18 polypeptide comprises the amino acid sequence set forth in SEQ ID NO: 81. In some embodiments, the IL-18 polypeptide of the Act-IL-18 polypeptide comprises the amino acid sequence set forth in SEQ ID NO: 82. In some embodiments, the IL-18 polypeptide of the Act-IL-18 polypeptide comprises the amino acid sequence set forth in SEQ ID NO: 83.

TABLE 3
IL-18 polypeptides

| SQ ID | | |
|---|---|---|
| NO: | Modification | Sequence |
| 1 | Native sequence | YFGKLESKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 2 | E6K, C38A, K53A, | YFGKLKSKLS VIRNLNDQVL FIDQGNRPLF |
| | C68A, E85C | EDMTDSDARD NAPRTIFIIS MYADSQPRGM |
| | | AVTISVKAEK ISTLSCENKI ISFKCMNPPD |
| | | NIKDTKSDII FFQRSVPGHD NKMQFESSSY |
| | | EGYFLACEKE RDLFKLILKK EDELGDRSIM |
| | | FTVQNED |
| 3 | E6K, V11I, C38A, | YFGKLKSKLS IIRNLNDQVL FIDQGNRPLF |
| | K53A, T63A, C68A, | EDMTDSDARD NAPRTIFIIS MYADSQPRGM |
| | C76A, E85C, C127A | AVAISVKAEK ISTLSAENKI ISFKCMNPPD |
| | | NIKDTKSDII FFQRSVPGHD NKMQFESSSY |
| | | EGYFLAAEKE RDLFKLILKK EDELGDRSIM |
| | | FTVQNED |
| 4 | E6K, C38A, K53A, | YFGKLKSKLS VIRNLNDQVL FIDQGNRPLF |
| | C68A, M86C | EDMTDSDARD NAPRTIFIIS MYADSQPRGM |
| | | AVTISVKAEK ISTLSCENKI ISFKECNPPD |
| | | NIKDTKSDII FFQRSVPGHD NKMQFESSSY |
| | | EGYFLACEKE RDLFKLILKK EDELGDRSIM |
| | | FTVQNED |
| 5 | E6K, V11I, C38A, | YFGKLKSKLS IIRNLNDQVL FIDQGNRPLF |
| | K53A, T63A, C68A, | EDMTDSDARD NAPRTIFIIS MYADSQPRGM |
| | C76A, M86C, C127A | AVAISVKAEK ISTLSAENKI ISFKECNPPD |
| | | NIKDTKSDII FFQRSVPGHD NKMQFESSSY |
| | | EGYFLAAEKE RDLFKLILKK EDELGDRSIM |
| | | FTVQNED |
| 6 | E6K, V11I, C38A, | YFGKLKSKLS IIRNLNDQVL FIDQGNRPLF |
| | K53A, T63A, C68A, | EDMTDSDARD NAPRTIFIIS MYADSQPRGM |
| | C76A, D98C, C127A | AVAISVKAEK ISTLSAENKI ISFKEMNPPD |
| | | NIKDTKSCII FFQRSVPGHD NKMQFESSSY |
| | | EGYFLAAEKE RDLFKLILKK EDELGDRSIM |
| | | FTVQNED |
| 7 | E6K, C38A, K53A, | YFGKLKSKLS VIRNLNDQVL FIDQGNRPLE |
| | C68A, C76A, M86C, | EDMTDSDARD NAPRTIFIIS MYADSQPRGM |
| | C127A | AVTISVKAEK ISTLSAENKI ISFKECNPPD |
| | | NIKDTKSDII FFQRSVPGHD NKMQFESSSY |
| | | EGYFLAAEKE RDLFKLILKK EDELGDRSIM |
| | | FTVQNED |
| 8 | E6K, V11I, C38A, | YFGKLKSKLS IIRNLNDQVL FIDQGNRPLF |
| | K53A, T63A, C68A, | EDMTDSDARD NAPRTIFIIS MYADSQPRGM |
| | C76A, T95C, C127A | AVAISVKAEK ISTLSAENKI ISFKEMNPPD |
| | | NIKDCKSDII FFQRSVPGHD NKMQFESSSY |
| | | EGYFLAAEKE RDLFKLILKK EDELGDRSIM |
| | | FTVQNED |
| 9 | E6K, C38A, K53A, | YFGKLKSKLS VIRNLNDQVL FIDQGNRPLF |
| | C68A, D98C | EDMTDSDARD NAPRTIFIIS MYADSQPRGM |
| | | AVTISVKAEK ISTLSCENKI ISFKEMNPPD |
| | | NIKDTKSCII FFQRSVPGHD NKMQFESSSY |
| | | EGYFLACEKE RDLFKLILKK EDELGDRSIM |
| | | FTVQNED |
| 10 | E6K, C38A, K53A, | YFGKLKSKLS VIRNLNDQVL FIDQGNRPLF |
| | C68A, C76A, D98C, | EDMTDSDARD NAPRTIFIIS MYADSQPRGM |
| | C127A | AVTISVKAEK ISTLSAENKI ISFKEMNPPD |
| | | NIKDTKSCII FFQRSVPGHD NKMQFESSSY |
| | | EGYFLAAEKE RDLFKLILKK EDELGDRSIM |
| | | FTVQNED |
| 11 | E6K, V11I, C38A, | YFGKLKSKLS IIRNLNDQVL FIDQGNRPLF |
| | K53A, C76A, C127A | EDMTDSDARD NAPRTIFIIS MYADSQPRGM |
| | | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 12 | E6K, C38A, K53A, | YFGKLKSKLS VIRNLNDQVL FIDQGNRPLE |
| | T63A, C76A, C127A | EDMTDSDARD NAPRTIFIIS MYADSQPRGM |
| | | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 13 | E6K, K53A, T63N | YFGKLKSKLS VIRNLNDQVL FIDQGNRPLF |
| | | EDMTDSDCRD NAPRTIFIIS MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 14 | E6K, K53A, S50A, | YFGKLKSKLS VIRNLNDQVL FIDQGNRPLF |
| | T63N | EDMTDSDCRD NAPRTIFIIA MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 15 | E6K, K53A, S50H, | YFGKLKSKLS VIRNLNDQVL FIDQGNRPLF |
| | T63N | EDMTDSDCRD NAPRTIFIIH MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 16 | E6K, K53A, T63N, | YFGKLKSKLS VIRNLNDQVL FIDQGNRPLF |
| | S65A | EDMTDSDCRD NAPRTIFIIS MYADSQPRGM |
| | | AVNIAVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 17 | E6K, K53A, S50H | YFGKLKSKLS VIRNLNDQVL FIDQGNRPLF |
| | | EDMTDSDCRD NAPRTIFIIH MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 18 | E6K, C38A, K53A, | YFGKLKSKLS VIRNLNDQVL FIDQGNRPLF |
| | C68A | EDMTDSDARD NAPRTIFIIS MYADSQPRGM |
| | | AVTISVKAEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 19 | E6K, K53A, K79A | YFGKLKSKLS VIRNLNDQVL FIDQGNRPLF |
| | | EDMTDSDCRD NAPRTIFIIS MYADSQPRGM |
| | | AVTISVKCEK ISTLSCENAI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 20 | E6K, K53A, R104A | YFGKLKSKLS VIRNLNDQVL FIDQGNRPLF |
| | | EDMTDSDCRD VIRNLNDQVL MYADSQPRGM |
| | | AVTISVKCEK NAPRTIFIIS ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 21 | E6K, K53A, G108A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPAHD NKMQFESSSY |
| | | EGYFLACEKE RDLFKLILKK EDELGDRSIM |
| | | FTVQNED |
| 22 | E6K, K53A, H109A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPGAD NKMQFESSSY |
| | | EGYFLACEKE RDLFKLILKK EDELGDRSIM |
| | | FTVQNED |
| 23 | E6K, K53A, K112A | EDMTDSDCRD NAPRTIFIIS MYADSQPRGM |
| | | YFGKLKSKLS VIRNLNDQVL FIDQGNRPLF |
| | | AVTISVKCEK ISTLSCENKI ISFKEMNPPD |
| | | NIKDTKSDII FFQRSVPGHD NAMQFESSSY |
| | | EGYFLACEKE RDLFKLILKK EDELGDRSIM |
| | | FTVQNED |
| 24 | E6K, C38A, K53A, | YFGKLKSKLS VIRNLNDQVL FIDQGNRPLF |
| | T63A, C76A | EDMTDSDARD NAPRTIFIIS MYADSQPRGM |
| | | AVAISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 25 | E6K, C38Q, K53A, | YFGKLKSKLS VIRNLNDQVL FIDQGNRPLF |
| | T63A, C76A | EDMTDSDQRD NAPRTIFIIS MYADSQPRGM |
| | | AVAISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 26 | E6K, C38A, K53A, | YFGKLKSKLS VIRNLNDQVL FIDQGNRPLF |
| | T63A, C127A | EDMTDSDARD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 27 | E6K, C38Q, K53A, | YFGKLKSKLS VIRNLNDQVL FIDQGNRPLF |
| | T63A, C127A | EDMTDSDORD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 28 | E6K, C38A, K53A, | YFGKLKSKLS VIRNLNDQVL FIDQGNRPLF |
| | T63A, C76A, C127A | EDMTDSDARD NAPRTIFIIS MYADSQPRGM |
| | | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 29 | E6K, V11I, C38A, | YFGKLKSKLS IIRNLNDQVL FIDQGNRPLE |
| | K53A, T63A | EDMTDSDARD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 30 | E6K, V11I, C38A, | YFGKLKSKLS IIRNLNDQVL FIDQGNRPLF |
| (A.k.a. | K53A, T63A, C76A, | EDMTDSDARD NAPRTIFIIS MYADSQPRGM |
| C086). | C127A | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 31 | C38A, C76A, C127A | YFGKLESKLS VIRNLNDQVL FIDQGNRPLF |
| | | EDMTDSDARD NAPRTIFIIS MYKDSQPRGM |
| | | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 32 | C38A | YFGKLESKLS VIRNLNDQVL FIDQGNRPLF |
| | | EDMTDSDARD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 33 | E6K, C38A, K53A, | YFGKLKSKLS VIRNLNDQVL FIDQGNRPLF |
| | T63A | EDMTDSDARD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 34 | E06K, K53A, S55A | YFGKLKSKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 35 | Y01G, F02A, E06K, | GAGKLKSKLS VIRNLNDQVL FIDQGNRPLF |
| | M51G, K53A, D54A, | EDMTDSDCRD NAPRTIFIIS GYAAAQPRGM |
| | S55A, T63A | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 36 | K53A | YFGKLESKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 37 | S55A | YFGKLESKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 38 | E06K | YFGKLKSKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 39 | E06K, K53A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 40 | E06K, S55A | YFGKLKSKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 41 | K53A, S55A | YFGKLESKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 42 | E06K, K53A, S55A, | YFGKLKSKLS VIRNLNDQVL FIDQGNRPLF |
| | T63A | EDMTDSDCRD NAPRTIFIIS MYADAQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 43 | E06K, K53A, S55A, | GFGKLKSKLS VIRNLNDQVL FIDQGNRPLF |
| | Y01G | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 44 | E06K, K53A, S55A, | YAGKLKSKLS VIRNLNDQVL FIDQGNRPLF |
| | F02A | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 45 | E06K, K53A, S55A, | YFGKLKSKLS VIRNLNDQVL FIDQGNRPLF |
| | D54A | EDMTDSDCRD NAPRTIFIIS MYAAAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 46 | E06K, K53A, S55A, | YFGKLKSKLS VIRNLNDQVL FIDQGNRPLF |
| | M51G | EDMTDSDCRD NAPRTIFIIS GYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 47 | C38S, C68S, C76S, | YFGKLESKLS VIRNLNDQVL FIDQGNRPLF |
| | C127S | EDMTDSDSRD NAPRTIFIIS MYKDSQPRGM |
| | | AVTISVKSEK ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 48 | C38S, C68S, C76S, | YFGKLESKLS VIRNLNDQVL FIDQGNRPLF |
| | C127S, K70C | EDMTDSDSRD NAPRTIFIIS MYKDSQPRGM |
| | | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 49 | E06K, K53A, S55A, | YFGKLKSKLS VIRNLNDQVL FIDQGNRPLF |
| | C38S, C68S, C76S, | EDMTDSDSRD NAPRTIFIIS MYADAQPRGM |
| | C127S, K70C | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 50 | E06K, K53A, T63A | YFGKLKSKLS VIRNLNDQVL FIDQGNRPLF |
| | | EDMTDSDCRD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 51 | T63A | YFGKLESKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 52 | E06K, T63A | YFGKLKSKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 53 | K53A, T63A | YFGKLESKLS VIRNLNDQVL FIDQGNRPLE |
| | | EDMTDSDCRD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 54 | E06K, K53A, C38S, | YFGKLKSKLS VIRNLNDQVL FIDQGNRPLF |
| | C68S, C76S, C127S, | EDMTDSDSRD NAPRTIFIIS MYADSQPRGM |
| | K70C | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 55 | K53A, T63A, C38S, | YFGKLESKLS VIRNLNDQVL FIDQGNRPLF |
| | C68S, C76S, C127S, | EDMTDSDSRD NAPRTIFIIS MYADSQPRGM |
| | K70C | AVAISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 56 | E6K, K53A, C38S, | YFGKLKSKLS VIRNLNDQVL FIDQGNRPLF |
| | C76S, C127S | EDMTDSDSRD NAPRTIFIIS MYADSQPRGM |
| | | AVTISVKCEK ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 57 | E6K, C38S, K53A | YFGKLKSKLS VIRNLNDQVL FIDQGNRPLF |
| | | EDMTDSDSRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 58 | E6K, K53A, C38S, | YFGKLKSKLS VIRNLNDQVL FIDQGNRPLF |
| | C68S, C76S, C127S, | EDMTDSDSRD NAPRTIFIIS MYADSQPRGM |
| | K70C | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 59 | E6K, C38A, K53A | YFGKLKSKLS VIRNLNDQVL FIDQGNRPLF |
| | | EDMTDSDARD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 60 | E6K, C38Q, K53A | YFGKLKSKLS VIRNLNDQVL FIDQGNRPLF |
| | | EDMTDSDORD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 61 | E6K, C38A, K53A, | YFGKLKSKLS VIRNLNDQVL FIDQGNRPLF |
| | C76A | EDMTDSDARD NAPRTIFIIS MYADSQPRGM |
| | | AVTISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 62 | E6K, C38A, K53A, | YFGKLKSKLS VIRNLNDQVL FIDQGNRPLF |
| | C127A | EDMTDSDARD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLAAEKE RDLFKLILKKEDELGDRSIM |
| | | FTVQNED |
| 63 | E6K, C38A, K53A, | YFGKLKSKLS VIRNLNDQVL FIDQGNRPLF |
| | C76A, C127A | EDMTDSDARD NAPRTIFIIS MYADSQPRGM |
| | | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 64 | E6K, K53A, C38A, | YFGKLKSKLS VIRNLNDQVL |
| | S55A, T63A | EDMTDSDARD NAPRTIFIIS MYADAQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 65 | E6K, C38Q, K53A, | YFGKLKSKLS VIRNLNDQVL FIDQGNRPLF |
| | S55A, T63A | EDMTDSDORD NAPRTIFIIS MYADAQPRGM |
| | | AVAISVKCEK ISTLSCENKI ISFKEMNPPD |
| | | NIKDTKSDII FFQRSVPGHD NKMOFESSSY |
| | | EGYFLACEKE RDLFKLILKK EDELGDRSIM |
| | | FTVQNED |
| 66 | E6K, K53A, K84A | YFGKLKSKLS VIRNLNDQVL FIDQGNRPLE |
| | | EDMTDSDCRD NAPRTIFIIS MYADSQPRGM |
| | | AVTISVKCEK ISTLSCENKI ISFAEMNPPD |
| | | NIKDTKSDII FFQRSVPGHD NKMQFESSSY |
| | | EGYFLACEKE RDLFKLILKK EDELGDRSIM |
| | | FTVQNED |
| 67 | E6K, K53A, D98A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSAII FFQRSVPGHD NKMQFESSSY |
| | | EGYFLACEKE RDLFKLILKK EDELGDRSIM |
| | | FTVQNED |
| 79 | V11I, C38A, M51G, | YFGKLESKLS IIRNLNDQVL FIDQGNRPLF |
| (A.k.a. | K53A, C76A, C127A | EDMTDSDARD NAPRTIFIIS GYADSQPRGM |
| C146) | | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 80 | E6K, V11I, C38A, | YFGKLKSKLS IIRNLNDQVL FIDQGNRPLF |
| (A.k.a. | M51G, K53A, T63A, | EDMTDSDARD NAPRTIFIIS GYADSQPRGM |
| C183) | C76A, C127A | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 81 | N-terminal G, E6K, | GYFGKLKSKLSIIRNLNDQVLFIDQGNRPLFEDMTDSD |
| (A.k.a. | V11I, C38A, M51G, | ARDNAPRTIFIISGYADSQPRGMAVAISVKCEKISTLS |
| C192) | K53A, T63A, C76A, | AENKIISFKEMNPPDNIKDTKSDIIFFQRSVPGHDNKM |
| | C127A | QFESSSYEGYFLAAEKERDLFKLILKKEDELGDRSIMF |
| | | TVQNED |
| 82 | N-terminal G, E6K, | GYFGKLKSKLSIIRNLNDQVLFIDOGNRPLFEDMTDSD |
| (A.k.a. | V11I, C38A, K53A, | ARDNAPRTIFIISMYADSQPRGMAVAISVKCEKISTLS |
| C141) | T63A, C76A, C127A | AENKIISFKEMNPPDNIKDTKSDIIFFQRSVPGHDNKM |
| | | QFESSSYEGYFLAAEKERDLFKLILKKEDELGDRSIMF |
| | | TVQNED |
| 83 | N-terminal 4xG, E6K, | GGGGYFGKLKSKLSIIRNLNDQVLFIDQGNRPLFEDMT |
| (A.k.a. | V11I, C38A, K53A, | DSDARDNAPRTIFIISMYADSQPRGMAVAISVKCEKIS |
| C140) | T63A, C76A, C127A | TLSAENKIISFKEMNPPDNIKDTKSDIIFFQRSVPGHD |
| | | NKMQFESSSYEGYFLAAEKERDLFKLILKKEDELGDRS |
| | | IMFTVQNED |

### Additional Exemplary IL-18 Constructs

Also provided herein are IL-18 polypeptides which comprise the modifications to SEQ ID NO: 1 listed in the table below, each of which is assigned a Composition ID. In some embodiments, the IL-18 polypeptide of an Act-IL-18 polypeptide comprises the set of amino acid substitutions shown for any one of the constructs depicted below. In the constructs depicted below, each of the substitutions is listed using SEQ ID NO: 1 as a reference sequence. In some embodiments, the -18 polypeptide of an Act-IL-18 polypeptide comprises only the substitutions shown for a construct below relative to SEQ ID NO: 1 (i.e., the IL-18 polypeptide has only the indicated set of substitutions and the remaining residues are those set forth in SEQ ID NO: 1).

TABLE 4
Additional IL-18 Constructs

| Composition ID/Substitutions | | Composition ID/Substitutions | | Composition ID/Substitutions | |
|---|---|---|---|---|---|
| to SEQ ID NO: 1 | | to SEQ ID NO: 1 | | to SEQ ID NO: 1 | |
| C143 | V11I, C38A, | C156 | V11I, C38A, N41A, | C168 | V11I, C38A, C76A, |
| | K53A, C76A, | | K53A, C76A, | | S105K, C127A |
| | C127A | | C127A | | |
| C144 | V11I, C38A, | C157 | V11I, C38A, K53A, | C174 | K8L, E6K, V11I, |
| | K53A, T63A, | | C76A, C127A, | | C38A, K53A, T63A, |
| | C76A, C127A | | D132A | | C76A, C127A |
| C145 | V11I, C38A, | C158 | V11I, C38A, K53A, | C175 | E6K, V11I, C38A, |
| | K53A, S55A, | | C76A, G108A, | | I49E, K53A, T63A, |
| | C76A, C127A | | C127A | | C76A, C127A |
| C147 | V11I, C38A, | C159 | V11I, C38A, K53A, | C176 | E6K, V11I, C38A, |
| | K53A, D54A, | | C76A, H109A, | | I49M, K53A, T63A, |
| | C76A, C127A | | C127A | | C76A, C127A |
| C148 | F2A, V11I, C38A, | C160 | V11I, C38A, K53A, | C177 | E6K, V11I, C38A, |
| | K53A, C76A, | | C76A, D110A, | | I49R, K53A, T63A, |
| | C127A | | C127A | | C76A, C127A |
| C149 | V11I, E31A, | C161 | K8R, V11I, C38A, | C178 | E6K, V11I, C38A, |
| | C38A, K53A, | | C76A, Q103E, | | K53A, T63A, C76A, |
| | C76A, C127A | | C127A | | Q103R, C127A |
| C150 | V11I, T34A, | C162 | K8E, V11I, C38A, | C179 | E6K, K8E, V11I, |
| | C38A, K53A, | | C76A, Q103R, | | C38A, K53A, T63A, |
| | C76A, C127A | | C127A | | C76A, Q103R, C127A |
| C151 | V11I, D35A, | C163 | V11I, C38A, C76A, | C180 | E6K, V11I, C38A, |
| | C38A, K53A, | | Q103K, C127A | | K53A, T63A, C76A, |
| | C76A, C127A | | | | C127A, V153R |
| C152 | V11I, S36A, C38A, | C164 | V11I, C38A, S55H, | C181 | E6K, V11I, C38A, |
| | K53A, C76A, | | C76A, C127A | | K53A, T63A, C76A, |
| | C127A | | | | C127A, V153E |
| C153 | V11I, D37A, | C165 | V11I, C38A, S55R, | C182 | E6K, V11I, C38A, |
| | C38A, K53A, | | C76A, C127A | | K53A, T63A, C76A, |
| | C76A, C127A | | | | C127A, V153Y |
| C154 | V11I, E31A, | C166 | V11I, C38A, S55T, | C184 | E6R, V11I, C38A, |
| | D37A, C38A, | | C76A, C127A | | K53A, T63A, C76A, |
| | K53A, C76A, | | | | C127A |
| | C127A | | | | |
| C155 | V11I, C38A, | C167 | V11I, C38A, C76A, | C142 | Y1M, E6K, V11I, |
| | D40A, K53A, | | S105I, C127A | | C38A, K53A, T63A, |
| | C76A, C127A | | | | C76A, C127A |

### Exemplary Act-IL-18 Sequences and Controls

| | | | Clea- | |
|---|---|---|---|---|
| | IL-18 | | vage | |
| | se- | | and/or | |
| Con- | quence | Blocking | Linker | |
| struct | modifi- | Moiety | se- | Full construct |
| number | cations | Sequence | quence | sequence |
| C190 | E6K, | MAAEPVED | N/A | MAAEPVEDNCINFVAMK |
| | V11I, | NCINFVAM | | FIDNTLYFIAEDDENLE |
| | C38A, | KFIDNTLY | | SDYFGKLKSKLSIIRNL |
| | K53A, | FIAEDDEN | | NDQVLFIDQGNRPLFED |
| | T63A, | LESD | | MTDSDARDNAPRTIFII |
| | C76A, | (SEQ ID | | SMYADSQPRGMAVAISV |
| | C127A | NO: 89) | | KCEKISTLSAENKIISF |
| | (SEQ | | | KEMNPPDNIKDTKSDII |
| | ID | | | FFQRSVPGHDNKMQFES |
| | NO: | | | SSYEGYFLAAEKERDLF |
| | 30) | | | KLILKKEDELGDRSIMF |
| | | | | TVQNED |
| | | | | (SEQ ID NO: 68) |
| C127 | E6K, | MAAEPVED | SGGPGP | MAAEPVEDNCINFVAMK |
| | V11I, | NCINFVAM | AGMKGL | FIDNTLYFIAEDDENLE |
| | C38A, | KFIDNTLY | PGS | SDSGGPGPAGMKGLPGS |
| | K53A, | FIAEDDEN | (SEQ | YFGKLKSKLSIIRNLND |
| | T63A, | LESD | ID | QVLFIDQGNRPLFEDMT |
| | C76A, | (SEQ ID | NO: | DSDARDNAPRTIFIISM |
| | C127A | NO: 89) | 101, | YADSQPRGMAVAISVKC |
| | (SEQ | | Linker | EKISTLSAENKIISFKE |
| | ID | | X) | MNPPDNIKDTKSDIIFF |
| | NO: | | | QRSVPGHDNKMQFESSS |
| | 30) | | | YEGYFLAAEKERDLFKL |
| | | | | ILKKEDELGDRSIMFTV |
| | | | | QNED |
| | | | | (SEQ ID NO: 69) |
| C185 | E6K, | MAAEPVED | PLG | MAAEPVEDNCINFVAMK |
| | V11I, | NCINFVAM | | FIDNTLYFIAEDDENLE |
| | C38A, | KFIDNTLY | | SDPLGYFGKLKSKLSII |
| | K53A, | FIAEDDEN | | RNLNDQVLFIDQGNRPL |
| | T63A, | LESD | | FEDMTDSDARDNAPRTI |
| | C76A, | (SEQ ID | | FIISMYADSQPRGMAVA |
| | C127A | NO: 89) | | ISVKCEKISTLSAENKI |
| | (SEQ | | | ISFKEMNPPDNIKDTKS |
| | ID | | | DIIFFQRSVPGHDNKMQ |
| | NO: | | | FESSSYEGYFLAAEKER |
| | 30) | | | DLFKLILKKEDELGDRS |
| | | | | IMFTVQNED |
| | | | | (SEQ ID NO: 70) |
| C186 | YIM, | MAAEPVED | PLG | MAAEPVEDNCINFVAMK |
| | E6K, | NCINFVAM | | FIDNTLYFIAEDDENLE |
| | V11I, | KFIDNTLY | | SDPLGMFGKLKSKLSII |
| | C38A, | FIAEDDEN | | RNLNDQVLFIDQGNRPL |
| | K53A, | LESD | | FEDMTDSDARDNAPRTI |
| | T63A, | (SEQ ID | | FIISMYADSQPRGMAVA |
| | C76A, | NO: 89) | | ISVKCEKISTLSAENKI |
| | C127A | | | ISFKEMNPPDNIKDTKS |
| | | | | DIIFFQRSVPGHDNKMQ |
| | | | | FESSSYEGYFLAAEKER |
| | | | | DLFKLILKKEDELGDRS |
| | | | | IMFTVQNED |
| | | | | (SEQ ID NO: 71) |
| C187 | E6K, | MAAEPVED | PLGLAG | MAAEPVEDNCINFVAMK |
| | V11I, | NCINFVAM | (SEQ | FIDNTLYFIAEDDENLE |
| | C38A, | KFIDNTLY | ID | SDPLGLAGYFGKLKSKL |
| | K53A, | FIAEDDEN | NO: | SIIRNLNDQVLFIDQGN |
| | T63A, | LESD | 206) | RPLFEDMTDSDARDNAP |
| | C76A, | (SEQ ID | | RTIFIISMYADSQPRGM |
| | C127A | NO: 89) | | AVAISVKCEKISTLSAE |
| | (SEQ | | | NKIISFKEMNPPDNIKD |
| | ID | | | TKSDIIFFQRSVPGHDN |
| | NO: | | | KMQFESSSYEGYFLAAE |
| | 30) | | | KERDLFKLILKKEDELG |
| | | | | DRSIMFTVQNED |
| | | | | (SEQ ID NO: 72) |
| C172 | E6K, | SNIVPVLL | SGGPGP | YFGKLKSKLSIIRNLND |
| | V11I, | GPKLNHVA | AGMKGL | QVLFIDQGNRPLFEDMT |
| | C38A, | VELGKNVR | PG | DSDARDNAPRTIFIISM |
| | K53A, | LNCSALLN | (SEQ | YADSQPRGMAVAISVKC |
| | T63A, | EEDVIYWM | ID | EKISTLSAENKIISFKE |
| | C76A, | FGEENGSD | NO: | MNPPDNIKDTKSDIIFF |
| | C127A | PNIHEEKE | 100, | QRSVPGHDNKMQFESSS |
| | (SEQ | MRIMTPEG | Linker | YEGYFLAAEKERDLFKL |
| | ID | KWHASKVL | X) | ILKKEDELGDRSIMFTV |
| | NO: | RIENIGES | | QNESGGPGPAGMKGLPG |
| | 30) | NLNVLYNC | | SNIVPVLLGPKLNHVAV |
| | | TVASTGGT | | ELGKNVRLNCSALLNEE |
| | | DTKSFILV | | DVIYWMFGEENGSDPNI |
| | | RKAD | | HEEKEMRIMTPEGKWHA |
| | | (SEQ ID | | SKVLRIENIGESNLNVL |
| | | NO: 93) | | YNCTVASTGGTDTKSFI |
| | | | | LVRKADHHHHHH |
| | | | | (SEQ ID NO: 73) |
| C173 | E6K, | SNIVPVLL | PLGLAG | YFGKLKSKLSIIRNLND |
| | V11I, | GPKLNHVA | (SEQ | QVLFIDQGNRPLFEDMT |
| | C38A, | VELGKNVR | ID | DSDARDNAPRTIFIISM |
| | K53A, | LNCSALLN | NO: | YADSQPRGMAVAISVKC |
| | T63A, | EEDVIYWM | 206) | EKISTLSAENKIISFKE |
| | C76A, | FGEENGSD | | MNPPDNIKDTKSDIIFF |
| | C127A | PNIHEEKE | | QRSVPGHDNKMQFESSS |
| | (SEQ | MRIMTPEG | | YEGYFLAAEKERDLFKL |
| | ID | KWHASKVL | | ILKKEDELGDRSIMFTV |
| | NO: | RIENIGES | | QNEPLGLAGSNIVPVLL |
| | 30) | NLNVLYNC | | GPKLNHVAVELGKNVRL |
| | | TVASTGGT | | NCSALLNEEDVIYWMFG |
| | | DTKSFILV | | EENGSDPNIHEEKEMRI |
| | | RKAD | | MTPEGKWHASKVLRIEN |
| | | (SEQ ID | | IGESNLNVLYNCTVAST |
| | | NO: 93) | | GGTDTKSFILVRKADHH |
| | | | | HHHH |
| | | | | (SEQ ID NO: 74) |
| C189 | E6K, | SNIVPVLL | SGGPGP | YFGKLKSKLSIIRNLND |
| | V11I, | GPKLNHVA | AGMKGL | QVLFIDQGNRPLFEDMT |
| | C38A, | VELGKNVR | PG | DSDARDNAPRTIFIISM |
| | K53A, | LDCSALLN | (SEQ | YADSQPRGMAVAISVKC |
| | T63A, | EEDVIYWM | ID | EKISTLSAENKIISFKE |
| | C76A, | FGEEDGSD | NO: | MNPPDNIKDTKSDIIFF |
| | C127A | PNIHEEKE | 100, | QRSVPGHDNKMQFESSS |
| | (SEQ | MRIMTPEG | Linker | YEGYFLAAEKERDLFKL |
| | ID | KWHASKVL | X) | ILKKEDELGDRSIMFTV |
| | NO: | RIENIGES | | QNESGGPGPAGMKGLPG |
| | 30) | NLNVLYDC | | SNIVPVLLGPKLNHVAV |
| | | TVASTGGT | | ELGKNVRLDCSALLNEE |
| | | DTKSFILV | | DVIYWMFGEEDGSDPNI |
| | | RKAD | | HEEKEMRIMTPEGKWHA |
| | | (SEQ ID | | SKVLRIENIGESNLNVL |
| | | NO: 94) | | YDCTVASTGGTDTKSFI |
| | | | | LVRKADHHHHHH |
| | | | | (SEQ ID NO: 75) |
| C191 | E6K, | SNIVPVLL | PLGLAG | YFGKLKSKLSIIRNLND |
| | V11I, | GPKLNHVA | (SEQ | QVLFIDQGNRPLFEDMT |
| | C38A, | VELGKNVR | ID | DSDARDNAPRTIFIISM |
| | K53A, | LNCSALLN | NO: | YADSQPRGMAVAISVKC |
| | T63A, | EEDVIYWM | 206) | EKISTLSAENKIISFKE |
| | C76A, | FGEENGSD | | MNPPDNIKDTKSDIIFF |
| | C127A | PNIHEEKE | | QRSVPGHDNKMQFESSS |
| | (SEQ | MRIMTPEG | | YEGYFLAAEKERDLFKL |
| | ID | KWHASKVL | | ILKKEDELGDRSIMTVF |
| | NO: | RIENIGES | | QNESGGPLGLAGGSNIV |
| | 30) | NLNVLYNC | | PVLLGPKLNHVAVELGK |
| | | TVASTGGT | | NVRLNCSALLNEEDVIY |
| | | DTKSFILV | | WMFGEENGSDPNIHEEK |
| | | RKAD | | EMRIMTPEGKWHASKVL |
| | | (SEQ ID | | RIENIGESNLNVLYNCT |
| | | NO: 93) | | VASTGGTDTKSFILVRK |
| | | | | ADHHHHHH |
| | | | | (SEQ ID NO: 76) |
| C136 | E6K, | SNIVPVLL | GGSGG | YFGKLKSKLSIIRNLND |
| | V11I, | GPKLNHVA | (SEQ | QVLFIDQGNRPLFEDMT |
| | C38A, | VELGKNVR | ID | DSDARDNAPRTIFIISM |
| | K53A, | LNCSALLN | NO: | YADSQPRGMAVAISVKC |
| | T63A, | EEDVIYWM | 244) | EKISTLSAENKIISFKE |
| | C76A, | FGEENGSD | | MNPPDNIKDTKSDIIFF |
| | C127A | PNIHEEKE | | QRSVPGHDNKMQFESSS |
| | (SEQ | MRIMTPEG | | YEGYFLAAEKERDLFKL |
| | ID | KWHASKVL | | ILKKEDELGDRSIMFTV |
| | NO: | RIENIGES | | QNEGGSGGSNIVPVLLG |
| | 30) | NLNVLYNC | | PKINHVAVELGKNVRLN |
| | | TVASTGGT | | CSALLNEEDVIYWMFGE |
| | | DTKSFILV | | ENGSDPNIHEEKEMRIM |
| | | RKAD | | TPEGKWHASKVLRIENI |
| | | (SEQ ID | | GESNLNVLYNCTVASTG |
| | | NO: 93) | | GTDTKSFILVRKADHHH |
| | | | | HHH |
| | | | | (SEQ ID NO: 77) |
| C137 | E6K, | SNIVPVLL | GGGGSG | YFGKLKSKLSIIRNLND |
| | V11I, | GPKLNHVA | GGGSGG | QVLFIDQGNRPLFEDMT |
| | C38A, | VELGKNVR | GG | DSDARDNAPRTIFIISM |
| | K53A, | LNCSALLN | (SEQ | YADSQPRGMAVAISVKC |
| | T63A, | EEDVIYWM | ID | EKISTLSAENKIISFKE |
| | C76A, | FGEENGSD | NO: | MNPPDNIKDTKSDIIFF |
| | C127A | PNIHEEKE | 243) | QRSVPGHDNKMQFESSS |
| | (SEQ | MRIMTPEG | | YEGYFLAAEKERDLFKL |
| | ID | KWHASKVL | | ILKKEDELGDRSIMFTV |
| | NO: | RIENIGES | | QNEGGGGSGGGGSGGGG |
| | 30) | NLNVLYNC | | SNIVPVLLGPKLNHVAV |
| | | TVASTGGT | | ELGKNVRLNCSALLNEE |
| | | DTKSFILV | | DVIYWMFGEENGSDPNI |
| | | RKAD | | HEEKEMRIMTPEGKWHA |
| | | (SEQ ID | | SKVLRIENIGESNLNVL |
| | | NO: 93) | | YNCTVASTGGTDTKSFI |
| | | | | LVRKADHHHHHH |
| | | | | (SEQ ID NO: 78) |

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples that are given for illustration purposes only and are not intended to limit the disclosure in any way.

### Examples

### Example 1A—Recombinant Act-IL18 Expression and Purification

Recombinant Act-IL-18 variants provided herein can be prepared according to the protocols provided below.

### Soluble His-SUMO-IL18 Variants

E. coli BL21 (DE3) harboring a plasmid encoding a N-His-SUMO tagged IL-18 variant fusion is inoculated into 3 L LB culture medium and induced with 0.4 mM IPTG at 30° C. for 6 h. Cells are pelleted and cell lysis is done by sonication in lysis buffer: PBS, pH 7.4. Soluble protein is purified via Ni-NTA beads 6FF (wash 1 with: PBS, 20 mM imidazole, pH7.4; wash 2 with PBS, 50 mM Imidazole, pH7.4; elution with PBS, 500 mM imidazole, pH7.4).

Fractions containing the protein are pooled, dialyzed into PBS pH 7.4 and followed by SUMO digestion. Then the protein is two-step purified with Ni-NTA beads (continue with flow through sample) and gel filtration. Fractions containing the protein are pooled and QC is performed using analytical techniques, such as SDS-PAGE and analytical SEC.

### Insoluble His-SUMO-IL18 Variants

E. coli BL21 (DE3) harboring a plasmid encoding a N-His-SUMO tagged IL-18 variant fusion are inoculated into 10 L LB culture medium and induced with 0.4 mM IPTG at 30° C. for 6 h. Cells are pelleted and cell lysis is done by sonication in lysis buffer: PBS, 8 M urea, pH 7.4. Protein is purified via Ni-NTA beads 6FF (wash 1 with: PBS, 8 M urea, 20 mM imidazole, pH7.4; wash 2 with PBS, 8 M urea, 50 mM Imidazole, pH7.4; elution with PBS, 8 M urea, 500 mM imidazole, pH7.4).

Fractions containing the protein are pooled, dialyzed into PBS pH 7.4 and followed by SUMO digestion. Then the protein is purified with Ni-NTA beads (equilibrate column with PBS, 8 M urea, pH 7.4, wash with PBS, 8 M urea, pH 7.4, elution with PBS, 8 M urea, pH 7.4). Fractions containing the protein are pooled, dialyzed into PBS pH 7.4 and QC is performed using analytical techniques, such as SDS-PAGE and analytical SEC.

### Insoluble Tagless IL18 Variants

E. coli BL21 (DE3) harboring a plasmid encoding mIL-18 is inoculated into 2 L LB culture medium and induced with 0.4 mM IPTG at 30° C. for 6 h. Cells are pelleted and cell lysis was done by sonication in lysis buffer: 110 mM Tris, 1.1 M guanidine HCl, 5 mM DTT, pH 8.9. Protein as purified via Q Sepharose FF (balance buffer 20 mM MES, pH 7.0, elution with an increasing gradient from 0 to 1 M NaCl).

### Bicistronic System

A single colony of E. coli BL21 containing the plasmid (e.g., SEQ ID: 59) is used as an inoculum for 10 mL LB containing 25 μg/mL kanamycin sulfate and incubated overnight at 37° C. and 200 rpm. 1 mL of the preculture are used to inoculate 1 L autoinducing terrific broth containing 100 μg/mL kanamycin sulfate. The culture is incubated at 37° C. and 110 rpm for 4 h and then transferred to 15° C. for another 15 h. Cells are resuspended in 10-15 mL lysis buffer (100 mM HEPES, 1 mM EDTA, 5 mM DTT, 20 μg/mL lysozyme, 0.1 mg/mL DNase I, 1 mM PMSF, pH 7.5) and gently shaken at 4° C. for 1 h. Then the cells are lysed with sonication and the soluble protein fraction is obtained by centrifugation (16,000×g, 30 min, 4° C.) and filtration (0.2 μm membrane).

The supernatant is adjusted to ca. pH 7 and loaded on a tandem column system (2× SP CIEX+1×HiPrep DEAE FF 16/10, all from cytiva) using a 50 mL superloop (loading less than 30 mL lysate per run). The system is run with wash buffer (25 mM HEPES, 1 mM EDTA, 5 mM DTT, pH 7.0) and fractions containing the protein (second main peak) are collected and pooled.

The tandem columns are separated into their respective types. The DEAE columns were eluted with buffers E1 and E2 (25 mM Bus-Tris Propane HCl, pH 9.5 and 25 mM Bis-Tris Propane HCl, 1 M NaCl, pH 9.5 respectively) with a stepwise gradient. First, 100% E1 was run for 8 CV, followed by a gradient from 0% to 12% E2 over 5 CV and then keeping it at 12% for another 10 CV. This is followed by a gradient from 12% to 40% E2 over 5 CV and keeping it at 40% for another 5 CV. Fractions containing the protein (second main peak) are collected and pooled with the previous fractions. The SP columns are washed with the same method and discard, as no protein should be found in this elution.

The pooled samples are adjusted to pH 9.5 and loaded on a Mono Q (small scale) or Hitrap Q (large scale) column. Buffers used are E2 and E3 (25 mM Bis-Tris Propane HCl, 1.5 M Ammonium Sulfate, pH 9.5). The stepwise elution gradient starts at 8% E3 for 15 CV, increasing to 16% E3 over 5 CV and the increasing to 50% E3 over 3 CV. Fractions containing the protein are found in the second main peak.

The fractions containing the target protein are pooled and concentrated by diafiltration (10 kDa MWCO, less than 3500×g, 4° C.). The concentrated sample is loaded on a Superdex 75 equilibrated with buffer (20 mM potassium phosphate, 150 mM KCl, 1 mM DTT, pH 6.0). Fractions containing the target protein are collected, pooled and concentrated

### Example 1B—Expression and Purification of Selected Activatable IL-18 Polypeptides

Activatable IL-18 candidates with N-terminal propeptide attached were prepared according to the methods provided below.

### 1.1—Expression of Activatable IL-18 Candidate

Activatable IL-18 candidates were produced as an N-terminal fusion to N-His-SUMO-IL18. The gene was synthesized and cloned into plasmids. Plasmids were transformed into E. coli BL21 (DE3). Expression was performed in shake flasks with TB medium. The cells were grown at 37° C. until an OD600 of approximately 1.2 was reached, after which they were induced by 0.1 mM IPTG and cultured for another 20 hours at 18° C. Cells were harvested by centrifugation.

### 1.2—Purification of Activatable IL-18 Candidates

### 1.2.1—Cell Lysis

Cells were resuspended in lysis buffer (20 mM Tris/HCl, pH 8.0, 0.15 M NaCl, 10 mM Imidazole, 1 tablet of EDTA-free complete protease inhibitor (Roche, COEDTAF-RO) per liter production) at 100 mL buffer/L culture and disrupted twice with a homogenizer at 1000 bar. The lysate was cleared of debris by centrifugation at 40′000 g for 2×45 minutes, changing flask in between, and subsequent filtration through a 0.22 μm filter.

### 1.2.2—Affinity Purification and Endotoxin Removal

The lysate was loaded on Ni NTA resin (Cytiva, 17524802) pre-equilibrated with 20 mM Tris/HCl, pH 8.0, 0.15 M NaCl, 10 mM Imidazole, at 5 mL/min and washed with the same buffer for 5 CV. To remove endotoxins, the column was washed with 20 mM Tris/HCl, pH 8.0, 0.15 M NaCl, 10 mM Imidazole, 0.1% Triton X-114 at 10 mL/min for 30 CV. The column was washed with 20 mM Tris/HCl, pH 8.0, 0.15 M NaCl, 10 mM Imidazole, for 5 CV at 5 mL/min and the protein of interest eluted by linear increase of imidazole concentration. The column was then regenerated by 0.5M NaOH.

### 1.2.3—SUMO Digestion and Dialysis

To cleave the SUMO tag, SUMO protease was added to the elution pool at a w/w ratio of 1:250 (protein:SUMO enzyme) and incubated for 18 hours at 4° C. At the same time, the protein was dialyzed (20 mM Tris, pH 8.0, 150 mM NaCl), to reduce the imidazole concentration.

1.2.4—Purification by reverse IMAC

In order to remove the cleaved tag and the SUMO protease, the digested protein was flown through a Ni NTA resin column pre-equilibrated with 20 mM Tris/HCl, pH 8.0, 0.15 M NaCl, 10 mM Imidazole, at 5 mL/min. The flow-through was collected.

The flow-through was concentrated to 2.6 mg/mL and buffer exchanged into either 20 mM HEPES, 150 mM NaCl, 0.5 mM TCEP, 10% glycerol, pH7.5 or PBS, 10% glycerol, pH7.4. Proteins were stored at −70° C. until further quality controls.

### Example 1C

Act-IL-18 polypeptides were prepared using a mammalian expression system using methods well known in the field.

### Example 2-Cleavage of Activatable IL-18 Polypeptides

Cleavage of activatable IL-18 polypeptides was performed according to the following protocol: IL-18 candidate (activatable or non-cleavable controls) were incubated with either MMP2 (SIGMA, PF023), MMP7 (SIGMA, CC1059) or MMP9 (SIGMA, PF024) diluted in MMP buffer MMP (25 mM TRIS, 10 mM CaCl₂), 0.05% Brij25, pH 7.5) at a final concentration of 1 ug/ml for MMP2 and MMP9 and 10 ug/ml for MMP7. Controls of proteins without enzymes were incubated in the same conditions. After 20 hours incubations, samples were collected for SDS gel analysis.

For the candidates that presented a C-terminal masking domain, a further step of cleaning by Nickel beads was performed to remove the cleaved, histidine tagged masking domain and the non-cleaved proteins. Briefly, protein and enzyme solutions were incubated with an excess of Nickel beads (at least 10 uL dry beads for every expected 40 ug of protein) for 30 minutes in shaking conditions. The flow-through was collected and bounded residues were eluted from the beads by incubation with 20 mM TRIS, 150 mM NaCl, 500 mM Imidazole, pH 8.

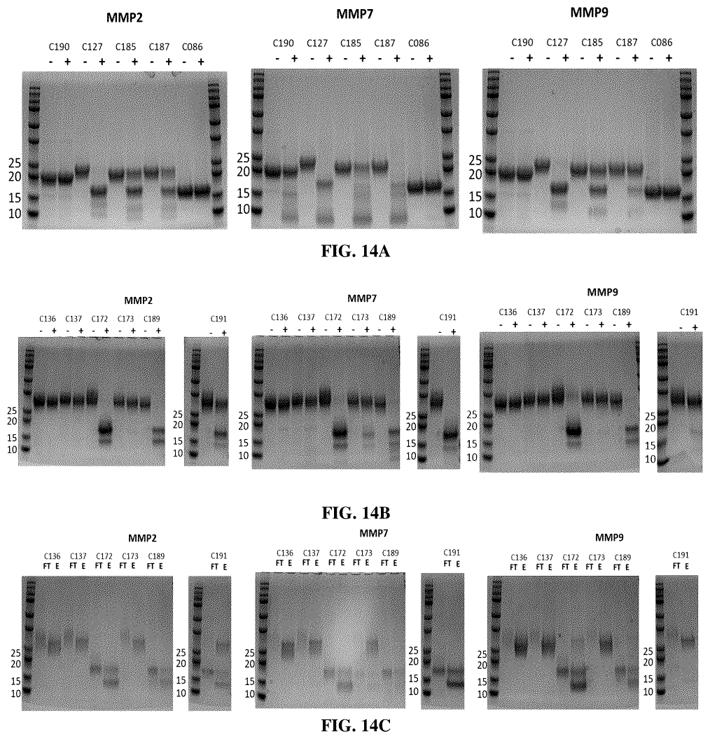

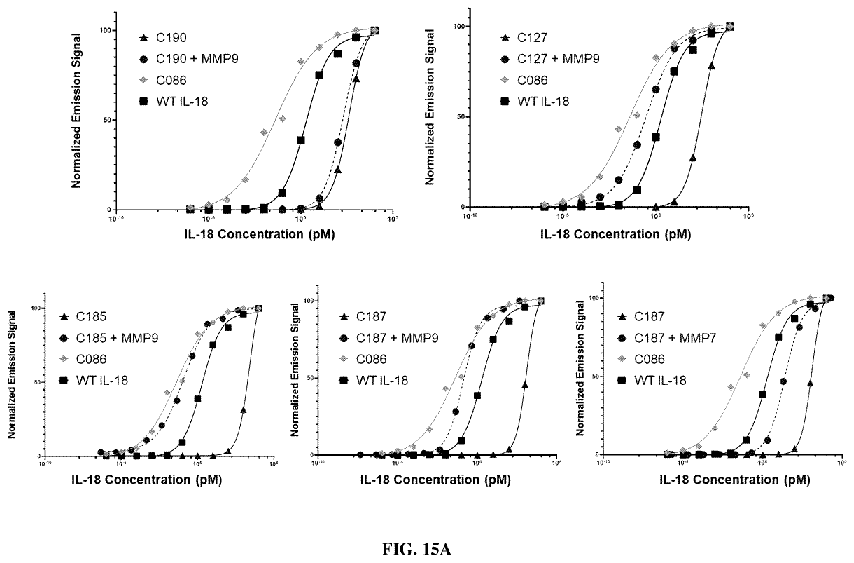

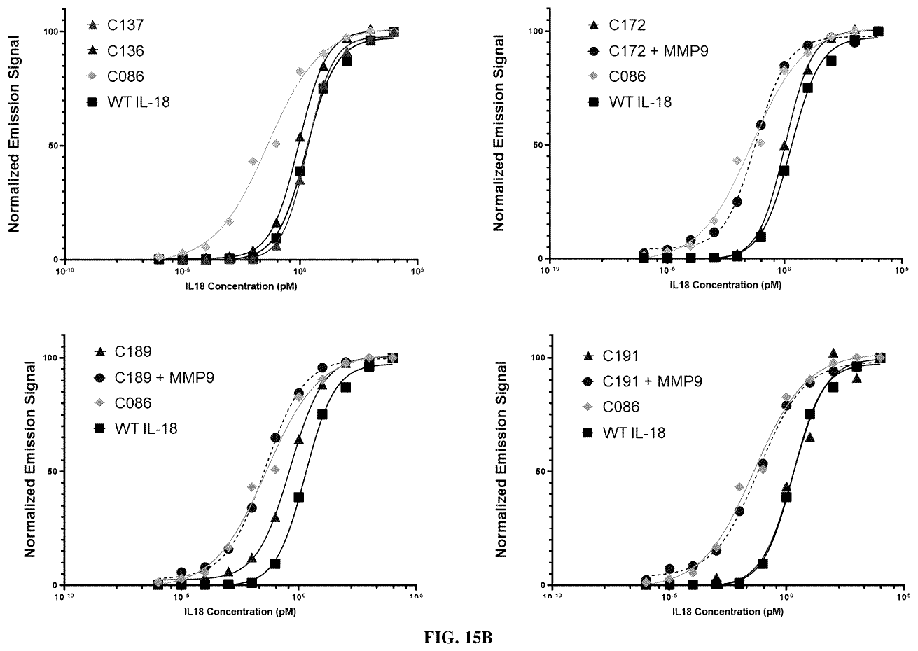

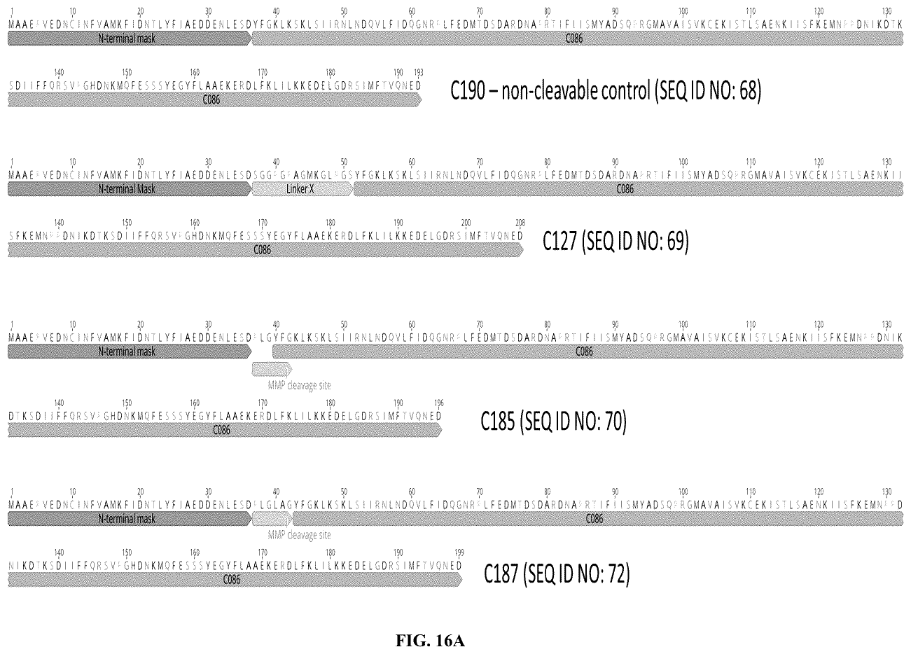

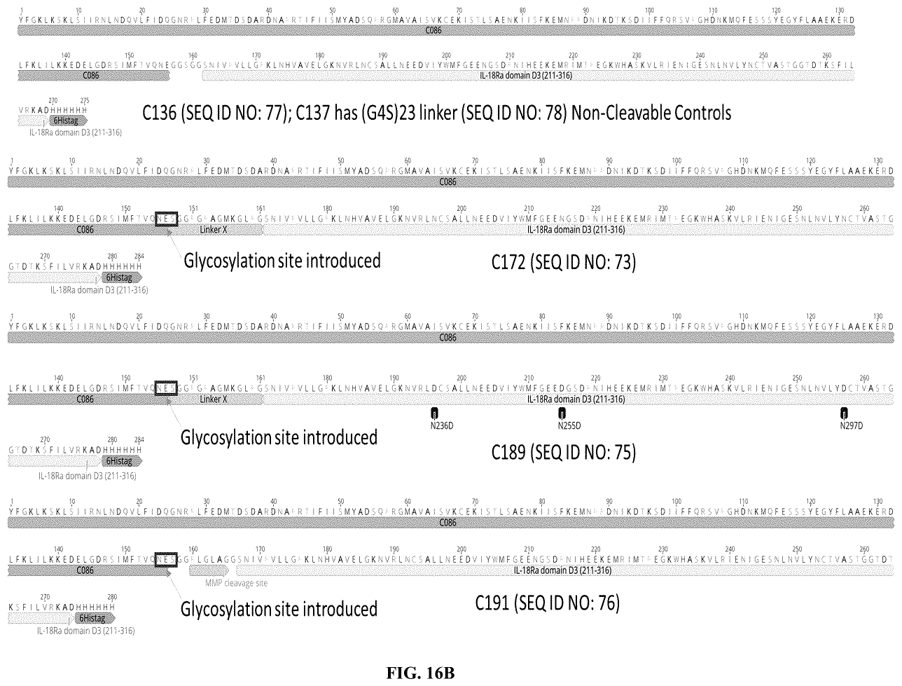

FIGS. 14A-C show resulting SDS-PAGE gels from cleavage experiments performed on the indicated IL-18 molecules. FIG. 14A shows resulting digestion of N-terminal masked candidates by MMP2, MMP7, and MMP9, with protease treated samples indicated with a (+) and untreated samples indicated with (−). Variant C127 showed efficient cleavage by each enzyme, C185 showed moderate cleavage by each enzyme, and C187 showed moderate cleavage by MMP2 and MMP7 but moderate cleavage by MMP9, whereas base IL-18 C086 and non-cleavable control C190 remained intact in all conditions. FIG. 14B shows resulting digestion (unpurified) of C-terminal masked candidates by MMP2, MMP7, and MMP9, with protease treated samples indicated with a (+) and untreated samples indicated with (−). Variant C136 showed no cleavage by any enzyme, C137 showed no cleavage by any enzyme, C172 showed efficient cleavage by all enzymes, C173 showed some cleavage by MMP7, C189 showed efficient cleavage by all enzymes, and C191 showed moderate cleavage by MMP2, efficient cleavage by mMP7, and no/minimal cleavage by MMP9. FIG. 14C shows resulting digestion of nickel purified flow through (FT) and eluate (E) of the C-terminal masked candidates (same sample as FIG. 14B).

### Example 3A-HEK-Blue IL18R Reporter Assay

An IL-18Rαβ positive HEK-Blue reporter cell line is used to determine binding of IL-18 variants to IL-18Rαβ and subsequent downstream signaling. The general protocol is outlined below.

5×104 cells HEK-Blue IL18R reporter cells (InvivoGen, #hkb-hmil18) are seeded into each well of a 96 well plate and stimulated with 0-100 nM of IL-18 polypeptide variants at 37° C. and 5% CO2. After 20 h incubation, 20 μL of cell culture supernatant is then taken from each well and mixed with 180 μL QUANTI-Blue media in a 96 well plate, incubated for 1 hour at 37° C. and 5% CO2. The absorbance signal at 620 nm is then measured on an Enspire plate reader with 680 and 615 nm as excitation and emission wavelengths, respectively. Half Maximal Effective dose (EC50) is calculated based on a variable slope, four parameter analysis using GraphPad PRISM software.

Act-IL-18 polypeptides provided herein display reduced or eliminated binding ability to stimulate IFNγ compared to WT IL-18 or the IL-18 polypeptide without the artificial terminal moiety. After cleavage, ability to stimulate IFNγ is restored, though may be altered relative to WT IL-18.

### Example 3B-HEK-Blue IL18R Reporter Assay-Results

The HEK-Blue IL-18R reporter assay described above was performed on activatable and control IL-18 polypeptides before and after treatment with indicated MMPs. The activity in the HEK-Blue IL18R assays is provided in the table below.

| | HEK-blue | Fold potency |
|---|---|---|
| Molecule | EC50 (nM) | loss vs C086 |
| IL-18 WT | 1.89 | 45 |
| C086 (SEQ ID | 0.04 | — |
| NO: 30) | | |
| C190 | 403.5 | 9580 |
| C190 + MMP9 | 197.5 | 4689 |
| C127 | 294.7 | 6997 |
| C127 + MMP9 | 0.3007 | 7 |
| C185 | 2565 | 60897 |
| C185 + MMP9 | 0.1131 | 3 |
| C187 | 1239 | 29416 |
| C187 + MMP9 | 0.1357 | 3 |
| C187 + MMP7 | 24.27 | 576 |
| C136 | 0.8837 | 21 |
| C137 | 2.084 | 49 |
| C172 | 1.093 | 26 |
| C172 + MMP9 | 0.06126 | 1 |
| C189 | 0.4298 | 10 |
| C189 + MMP9 | 0.03635 | 1 |
| C191 | 2.015 | 48 |
| C191 + MMP9 | 0.06723 | 2 |

### Example 3C

The HEK-Blue IL-18R reporter assay described above was also performed on additional IL-18 polypeptides which can be incorporated into Act-IL-18 polypeptides as provided herein. It is expected that the IL-18 polypeptides provided below would behave similarly to C086 (SEQ ID NO: 30) when converted to Act-IL-18 polypeptides as those otherwise provided herein.

| SEQ ID NO: or | Sequence | EC₅₀ |
|---|---|---|
| Composition ID | modifications | (pM) |
| 1 | Native sequence | 3.33 |
| 34 | E6K, K53A, S55A | 272.5 |
| 39 | E6K, K53A | 0.72 |
| 42 | E6K, K53A, S55A, T63A | 0.79 |
| 50 | E6K, K53A, T63A | 1.77 |
| 54 | E6K, C38S, K53A, C68S, K70C, C76S, C127S | 9.12 |
| 56 | E6K, K53A, C38S, C76S, C127S | 3.73 |
| 57 | E6K, C38S, K53A | 0.86 |
| 30 | E6K, V11I, C38A, K53A, T63A, C76A, C127A | 0.034 |
| 62 | E6K, C38A, K53A, C127A | 0.17 |
| 60 | E6K, C38Q, K53A | 0.203 |
| 59 | E6K, C38A, K53A | 0.268 |
| 57 | E6K, C38S, K53A | 0.53 |
| 57 | E6K, C38S, K53A | 0.53 |
| C143 | V11I, C38A, K53A, C76A, C127A | 0.98 |
| C144 | V11I, C38A, K53A, T63A, C76A, C127A | 0.17 |
| C145 | V11I, C38A, K53A, S55A, C76A, C127A | 3.63 |
| C146 | V11I, C38A, M51G, K53A, C76A, C127A | 0.8 |
| C147 | V11I, C38A, K53A, D54A, C76A, C127A | 1 |
| C148 | F2A, V11I, C38A, K53A, C76A, C127A | 7.28 |
| C149 | V11I, E31A, C38A, K53A, C76A, C127A | 6.6 |
| C150 | V11I, T34A, C38A, K53A, C76A, C127A | 0.7 |
| C151 | V11I, D35A, C38A, K53A, C76A, C127A | 13.12 |
| C152 | V11I, S36A, C38A, K53A, C76A, C127A | 0.25 |
| C153 | V11I, D37A, C38A, K53A, C76A, C127A | 14.12 |
| C154 | V11I, E31A, D37A, C38A, K53A, C76A, C127A | 11.95 |
| C155 | V11I, C38A, D40A, K53A, C76A, C127A | 0.52 |
| C156 | V11I, C38A, N41A, K53A, C76A, C127A | 11.7 |
| C157 | V11I, C38A, K53A, C76A, C127A, D132A | 1.95 |
| C158 | V11I, C38A, K53A, C76A, G108A, C127A | 15.56 |
| C159 | V11I, C38A, K53A, C76A, H109A, C127A | 19.5 |
| C160 | V11I, C38A, K53A, C76A, D110A, C127A | 2.02 |
| C161 | K8R, V11I, C38A, C76A, Q103E, C127A | 2.01 |
| C162 | K8E, V11I, C38A, C76A, Q103R, C127A | 2.3 |
| C163 | V11I, C38A, C76A, Q103K, C127A | 1.5 |
| C164 | V11I, C38A, S55H, C76A, C127A | 3.14 |
| C165 | V11I, C38A, S55R, C76A, C127A | 1.91 |
| C166 | V11I, C38A, S55T, C76A, C127A | 4.73 |
| C167 | V11I, C38A, C76A, S105I, C127A | 5.37 |
| C168 | V11I, C38A, C76A, S105K, C127A | 7.73 |
| C174 | K8L, E6K, V11I, C38A, K53A, T63A, C76A, C127A | 0.29 |
| C176 | E6K, V11I, C38A, I49M, K53A, T63A, C76A, C127A | 0.07 |
| C177 | E6K, V11I, C38A, I49R, K53A, T63A, C76A, C127A | 0.04 |
| C178 | E6K, V11I, C38A, K53A, T63A, C76A, Q103R, C127A | 0.26 |
| C179 | E6K, K8E, V11I, C38A, K53A, T63A, C76A, Q103R, | 0.4 |
| | C127A | |
| C181 | E6K, V11I, C38A, K53A, T63A, C76A, C127A, V153E | 0.1 |
| C182 | E6K, V11I, C38A, K53A, T63A, C76A, C127A, V153Y | 0.08 |
| C183 | E6K, V11I, C38A, M51G, K53A, T63A, C76A, C127A | 0.1 |
| C184 | E6R, V11I, C38A, K53A, T63A, C76A, C127A | 0.04 |
| C140 | E6K, V11I, C38A, K53A, T63A, C76A, C127A | 2.5 |
| C141 | E6K, V11I, C38A, K53A, T63A, C76A, C127A | 1.68 |
| C142 | Y1M, E6K, V11I, C38A, K53A, T63A, C76A, C127A | 0.02 |
| C192 | E6K, V11I, C38A, M51G, K53A, T63A, C76A, C127A | 13.99 |

### Example 4-Stability Assessment of IL-18s

The stability of activatable and control HL-18 polypeptides was assessed using nano differential scanning fluorimity (nanoDSF). Activatable HL-18 polypeptide constructs with either the Propeptide or HL-18 receptor D3 subunit attached showed enhanced stability compared to C086. Conversely, HL-18 polypeptides with a short extension peptide attached to the N-terminus showed lower stability.

| | | | ΔTm |
|---|---|---|---|
| | | Tm | VS |
| Sample | Protein | 550 nm | C086 |
| name | Description | (° C.) | (° C.) |
| C086 | C086 | 54.4 | |
| C190 | Propeptide-C086 | 62.2 | 7.8 |
| C172 | C086-LinkerX- | 61 | 6.6 |
| | RaD3 | | |
| C140 | GGGG-C086 | 51.3 | −3.1 |
| C141 | G-C086 | 52 | −2.4 |
| C142 | C086_Y1M | 54.9 | 0.5 |

In some instances, IL-18 polypeptides were subject to treatment with MMP or in MMP buffer without MMP according to the following general protocol: 100 uL samples of the indicated IL-18 at approximately 1 mg/mL were mixed with 100 uL of MMP-2 at 2 ug/mL in an MMP assay buffer (25 mM TRIS, 10 mM CaCl₂, 0.05% Brij 25, pH 7.5). Samples were incubated 16 hours in shaking conditions at 24° C.

| Sample | | | Tm 550 nm |
|---|---|---|---|
| name | Protein | Treatment | (° C.) |
| C086 | C086 | — | 54.4 |
| C086 buffer | | +MMP2 buffer | 50.2 |
| C086 mmp | | 0 | 50.2 |
| C190 | Propeptide-C086 | — | 62.2 |
| C190 buffer | | +MMP2 buffer | 57.4 |
| C190 mmp | | 0 | 57.3 |
| C127 | Propeptide-LinkerX- | — | 63.4 |
| C127 buffer | C086 | +MMP2 buffer | 58 |
| C127 mmp | | 0 | 50.9 |
| C136 | C086-2xGGS-RaD3 | — | 61.5 |
| C136 buffer | | +MMP2 buffer | 58 |
| C136 mmp | | 0 | 57.4 |
| C137 | C086-3xG4S-RaD3 | — | 62.6 |
| C137 buffer | | +MMP2 buffer | 57.2 |
| C137 mmp | | 0 | 57.2 |
| C172 | C086-LinkerX-RaD3 | — | 61 |
| C172 buffer | | +MMP2 buffer | 56.3 |
| C172 mmp | | 0 | 70.5 |
| C173 | C086-PLGLAG-RaD3 | — | 60 |
| C173 buffer | | +MMP2 buffer | 56.9 |
| C173 mmp | | 0 | 56.7 |

### Example 5A-Conjugation of Act-IL-18 Polypeptides

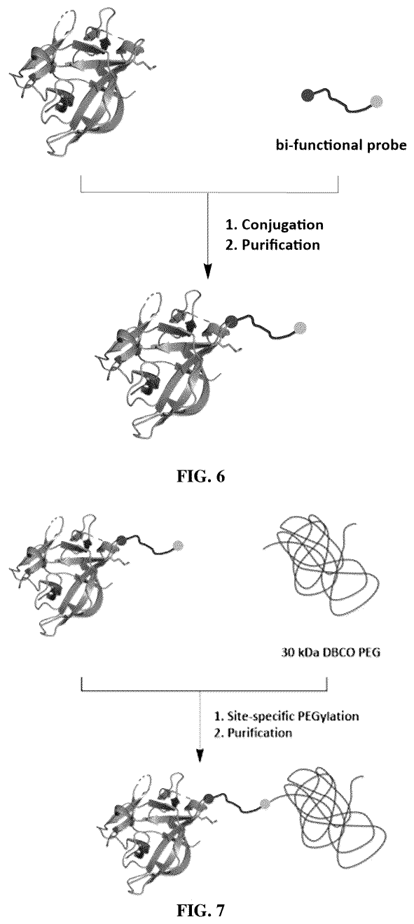

An Act-IL-18 polypeptide as provided herein is conjugated to a PEG functionality. In some cases, the PEG is attached via a bifunctional linker which first attaches to a desired residue of the Act-IL-18 polypeptide (e.g., E85C or another suitable naturally occurring cysteine, such as C68) or a cysteine residue which has been incorporated at a desired site, such as residue 86 or 98). Once attached to the IL-18 polypeptide, the second functionality of the bifunctional linker is used to attach the PEG moiety. An exemplary schematic of such a process is shown in FIG. 6. In some instances, a bifunctional linker as shown in FIG. 6 is not required because the IL-18 polypeptide will already comprise the desired conjugation handle for attachment of the PEG or other group. An exemplary protocol on a recombinant IL-18 variant provided herein is described below.

Conjugation-Recombinant IL-18 is stored at a concentration of 2.4 mg/mL at −80° C. in potassium phosphate buffer (pH 7.0) containing 50 mM KCl and 1 mM DTT. The sample is thawed on ice yielding a clear solution. The protein solution is diluted in PBS, pH 7.4. A clear solution is obtained at a concentration of ˜0.4 mg/mL.

The protein solution is dialyzed against PBS, pH 7.4 (twice against 600 mL for 2 h and once against 800 mL for 18 h). After dialysis, a clear solution is obtained with no sign of precipitation. Protein concentration is obtained using UV absorbance at 280 nm and by BCA protein assay.

A stock solution of bi-functional probe (bromoacetamido-PEG5-azide, CAS: 1415800-37-1) in water is prepared at a concentration of 20 mM. 500 μL of the protein solution are mixed with 25 μL of probe solution. pH was adjusted to 7.5 and it was let to react for 3 h at 20° C.

The progress of the synthesis is monitored by reverse-phase HPLC using a gradient of 5 to 30% (2.5 min) and 30 to 75% (7.5 min) CH3CN with 0.1% TFA (v/v) on a Aeris WIDEPORE C18 200 Å column (3.6 μm, 150×4.6 mm) at a flow rate of 1 mL/min at 40° C. and by MALDI-TOF MS.

Purification—In some cases, ion-exchange chromatography is used to purify the conjugated protein. To remove the excess of probe, the reaction mixture (volume is around 500 μL) is flowed through a Hi-Trap-G-FF-1 mL column using 25 mM Tris (pH 7.4) as the buffer. The column is eluted with a linear gradient of 0-0.35 M NaCl in the same buffer. The fractions containing the target protein are gathered, buffer exchanged (25 mM Tris, pH 7.4, 75 mM NaCl, 5% glycerol) and concentrated at 0.4 mg/mL. The concentration of purified protein is determined by UV absorbance at 280 nm and by BCA protein assay. The protein solution is kept at −80° C.

Characterization—The purity and identity of the recombinant protein from commercial source and the conjugated protein is confirmed by analytical SEC, HPLC and MALDI-TOF MS.

The Act-IL-18 polypeptide can then be further conjugated to an additional group, such as a polymer or an additional polypeptide.

### Example 5B—PEGylation of Act-IL-18 Polypeptides

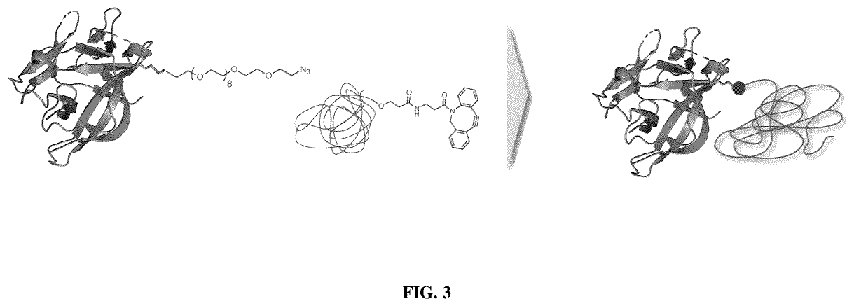

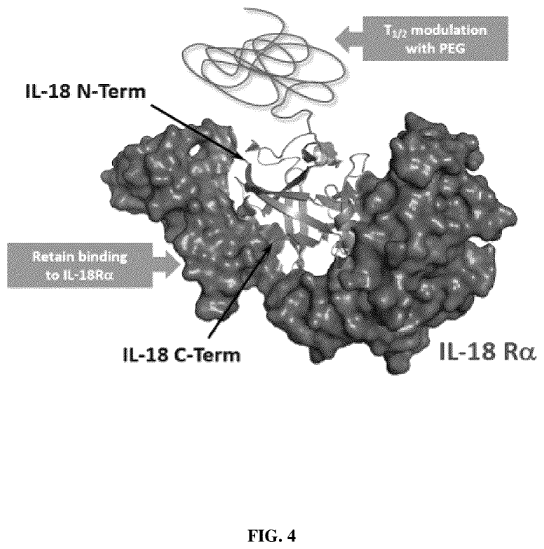

After conjugation of the bifunctional linker as described in Example 5A and as shown in FIG. 7, the Act-IL-18 polypeptide can be covalently linked with a PEG group. An exemplary schematic of this process is shown in FIG. 7. An exemplary protocol of the conjugation reaction between a PEG and a suitably activated IL-18 polypeptide is provided below. Additionally, the protocol below can be used to covalently link a desired PEG group to a Act-IL-18 polypeptide which incorporates a conjugation handle directly during the preparation of the Act-IL-18 polypeptide (e.g., during the synthesis of a synthetic IL-18 polypeptide). An exemplary schematic of such a process is shown in FIG. 3. A resulting Act-IL-18 polypeptide with polymer attached is depicted in FIG. 4, which is predicted to have an extended half-life.

Conjugation—Recombinant Act-IL-18 polypeptide comprising SEQ ID NO: 59 with an artificial terminal moiety as provided herein is stored at −80° C. in PBS (pH 7.4) containing 75 mM NaCl and 5% (v/v) glycerol. Prior to PEGylation reaction, the sample is thawed on ice yielding a clear solution. 200 μL of the protein solution (0.4 mg/mL) are mixed with 2.0 mg of DBCO-polyethylene glycol polymer. It is let to react overnight at 20° C.

The progress of the synthesis is monitored by reverse-phase HPLC using a gradient of 5 to 30% (2.5 min) and 30 to 75% (7.5 min) CH3CN with 0.1% TFA (v/v) on a Aeris WIDEPORE C4 200 Å column (3.6 μm, 150×4.6 mm) at a flow rate of 1 mL/min at 40° C. and by MALDI-TOF MS.

Purification—To remove the excess of PEG, the reaction mixture is diluted with Tris buffer (25 mM, pH 7.4) and flowed through a Hi-Trap-Q-FF column using 25 mM Tris (pH 7.4) as the buffer. The column is eluted with a linear gradient of 0-0.35 M NaCl in the same buffer. The fractions containing the target protein are gathered, buffer exchanged (25 mM Tris, pH 7.4, 75 mM NaCl, 5% glycerol) and concentrated at 0.04 mg/mL. The concentration of purified protein is determined by BCA protein assay. The protein solution is kept at −80° C.

Characterization—The purity and identity of the conjugated protein is confirmed by HPLC and MALDI-TOF MS.

Using an analogous protocol, the Act-IL-18 polypeptide could also be attached to another polypeptide, such as a suitable activated antibody.

### Example 6-Characterization of Act-IL-18 Polypeptides

Act-IL-18 polypeptides provided herein are subject to a series of analytical experiments to characterize the compositions. The Act-IL-18 polypeptides are analyzed by HPLC to determine the degree of uniformity in the compositions. The Act-IL-18 polypeptide compositions are also analyzed by MALDI-MS to determine the MW and distribution of molecular weights of the compositions. The Act-IL-18 polypeptide compositions are further analyzed by circular dichroism to compare the folding of the Act-IL-18 polypeptide compositions compared to wild type IL-18.

### Example 7-Formulation of Act-IL-18 Polypeptides

Lyophilized Act-IL-18 polypeptides are suspended in a solution comprising 10-50 mM Histidine buffer, 5-10% trehalose, 0.02% tween. Lyophilized Act-IL-18 polypeptide can also be resuspended in other suitable or appropriate buffers, such as PBS (pH 7.4) with mannitol (e.g., 50 mg/mL) and tween (e.g., 0.02%).

### Example 8-IL-18 SPR Measurements

The interaction of the wild type and of Act-IL-18 polypeptides with human IL-18 receptor subunits are measured with Surface Plasmon Resonance (SPR) technology. Anti-human IgG antibodies are bound by amine coupling onto a CM5 chip to capture 6 μg/mL of Fc fused human IL-18Rα, 6 μg/mL of Fc fused human IL-18Rβ, or 2 μg/mL of Fc fused human IL-18BP isoform a (IL-18BPa) for 30 min before capture. In other settings, 6 μg/mL of alpha and beta IL-18 receptors are mixed and pre-incubated for 30 min before capture of the alpha/beta heterodimer IL-18 receptor.

The kinetic binding of the IL-18 analytes are measured with a Biacore 8K instrument in two-fold serial dilutions starting at 1 μM down to 0.98 nM. Regeneration of the surface back to amine coupled anti IgG antibody is done after every concentration of analyte. To measure the protein association to the receptors, the samples are injected with a flow rate of 50 μL/min for 60 s, followed by 300 s buffer only to detect the dissociation. The used running buffer is 1×PBS with 0.05% Tween20. The relative response units (RU, Y-axis) are plotted against time (s, X-axis) and analyzed in a kinetic 1:1 binding model for the monomer receptor binding and for the binding to the IL-18BP. A kinetic heterogeneous ligand fit model is applied for the alpha/beta heterodimer binding.

Act-IL-18 polypeptides provided herein display reduced or eliminated binding to IL-18Rαβ, IL-Rα, and/or IL-18Rβ compared to WT IL-18 or the IL-18 polypeptide without the artificial terminal moiety. After cleavage, binding to these IL-18 receptor proteins is restored, though may be altered relative to WT IL-18.

### Example 9-IL-18BP Binding alphaLISA Assay

A human IL-18BP AlphaLISA Assay Kit is used to determine the binding affinity of each IL-18 variant for IL-18BP, which detected the presence of free form IL-18BP.

Sixteen three-fold serial dilutions of IL-18 analytes are prepared in aMEM medium supplemented with 20% FCS, Glutamax, and 25 μM β-mercaptoethanol in the presence of 5 ng/mL of His-tagged human IL-18BP. Final IL-18 analytes concentration range from 2778 nM to 0.2 pM.

After 1 hr incubation at room temperature, free IL-18BP levels are measured using a Human IFNγ AlphaLISA® Assay Kit. In a 384 well OPTIplate, 5 μL of 5× Anti-IL-18BP acceptor beads are added to 7.5 μL of an IL-18/IL-18BP mix. After 30 min incubation at room temperature with shaking, 5 μL of biotinylated Anti-IL-18BP antibodies are added to each well. The plate is incubated further for 1 hr at room temperature. Under subdued light, 12.5 μL of 2× streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (KD) is calculated based on a variable slope, four parameter analysis using GraphPad PRISM software.

Act-IL-18 polypeptides provided herein may display reduced or eliminated binding to IL-18BP with the artificial terminal moiety attached.

The experiment described above was also performed on IL-18 polypeptides which can be incorporated into Act-IL-18 polypeptides as provided herein to assess ability to bind to IL-18BP.

| SEQ ID NO: | | |
|---|---|---|
| or | | |
| Composition | Sequence | |
| ID | modifications | KD (nM) |
| 1 | Native Sequence | 0.67 |
| 34 | E06K, K53A, S55A | >1500 |
| 35 | Y01G, F02A, E06K, M51G, | 969.0 |
| | K53A, D54A, S55A, T63A | |
| 36 | K53A | 513.8 |
| 37 | S55A | 10.7 |
| 38 | E06K | 0.13 |
| 39 | E06K, K53A | 130.3 |
| 40 | E06K, S55A | 12.3 |
| 41 | K53A, S55A | 500.0 |
| 42 | E06K, K53A, S55A, T63A | 822.0 |
| 43 | E06K, K53A, S55A, Y01G | |
| 44 | E06K, K53A, S55A, F02A | >1000 |
| 45 | E06K, K53A, S55A, D54A | >1000 |
| 46 | E06K, K53A, S55A, M51G | >1000 |
| 47 | C38S, C68S, C76S, C127S | 0.03 |
| 48 | C38S, C68S, C76S, C127S, K70C | 0.21 |
| 49 | E06K, K53A, S55A, C38S, | >1000 |
| | C68S, C76S, C127S, K70C | |
| 50 | E06K, K53A, T63A | 339.8 |
| 51 | T63A | 2.59 |
| 52 | E06K, T63A | 0.83 |
| 53 | K53A, T63A | 198 |
| 54 | E06K, K53A, C38S, C68S, C76S, C127S, K70C | 446.0 |
| 55 | K53A, T63A, C38S, C68S, C76S, C127S, K70C | 913 |
| 56 | E6K, K53A, C38S, C76S, C127S | 435.5 |
| 57 | E6K, K53A, C38S | 50.2 |
| C143 | V11I, C38A, K53A, C76A, C127A | 8.86 |
| C144 | V11I, C38A, K53A, T63A, C76A, C127A | 0.66 |
| C145 | V11I, C38A, K53A, S55A, C76A, C127A | 9.74 |
| C146 | V11I, C38A, M51G, K53A, C76A, C127A | 373.30 |
| C147 | V11I, C38A, K53A, D54A, C76A, C127A | 25.77 |
| C148 | F2A, V11I, C38A, K53A, C76A, C127A | 57.21 |
| C149 | V11I, E31A, C38A, K53A, C76A, C127A | 0.64 |
| C150 | V11I, T34A, C38A, K53A, C76A, C127A | 1.24 |
| C151 | V11I, D35A, C38A, K53A, C76A, C127A | 2.88 |
| C152 | V11I, S36A, C38A, K53A, C76A, C127A | 1.12 |
| C153 | V11I, D37A, C38A, K53A, C76A, C127A | 4.55 |
| C154 | V11I, E31A, D37A, C38A, K53A, C76A, C127A | 2.12 |
| C155 | V11I, C38A, D40A, K53A, C76A, C127A | 0.74 |
| C156 | V11I, C38A, N41A, K53A, C76A, C127A | 18.47 |
| C157 | V11I, C38A, K53A, C76A, C127A, D132A | 13.70 |
| C158 | V11I, C38A, K53A, C76A, G108A, C127A | 1.24 |
| C159 | V11I, C38A, K53A, C76A, H109A, C127A | 0.55 |
| C160 | V11I, C38A, K53A, C76A, D110A, C127A | 0.71 |
| C161 | K8R, V11I, C38A, C76A, Q103E, C127A | 0.06 |
| C162 | K8E, V11I, C38A, C76A, Q103R, C127A | 0.85 |
| C163 | V11I, C38A, C76A, Q103K, C127A | 0.05 |
| C164 | V11I, C38A, S55H, C76A, C127A | 0.08 |
| C165 | V11I, C38A, S55R, C76A, C127A | 0.15 |
| C166 | V11I, C38A, S55T, C76A, C127A | 0.02 |
| C167 | V11I, C38A, C76A, S105I, C127A | 0.04 |
| C168 | V11I, C38A, C76A, S105K, C127A | 0.05 |
| C174 | K8L, E6K, V11I, C38A, K53A, T63A, C76A, C127A | 0.14 |
| C176 | E6K, V11I, C38A, I49M, K53A, T63A, C76A, C127A | 25.84 |
| C177 | E6K, V11I, C38A, I49R, K53A, T63A, C76A, C127A | >2800 |
| C178 | E6K, V11I, C38A, K53A, T63A, C76A, Q103R, C127A | >2800 |
| C179 | E6K, K8E, V11I, C38A, K53A, T63A, C76A, Q103R, | >2800 |
| | C127A | |
| C180 | E6K, V11I, C38A, K53A, T63A, C76A, C127A, V153R | |
| C181 | E6K, V11I, C38A, K53A, T63A, C76A, C127A, V153E | >2800 |
| C182 | E6K, V11I, C38A, K53A, T63A, C76A, C127A, V153Y | >2800 |
| C183 | E6K, V11I, C38A, M51G, K53A, T63A, C76A, C127A | >2800 |
| C184 | E6R, V11I, C38A, K53A, T63A, C76A, C127A | 5.46 |
| C140 | E6K, V11I, C38A, K53A, T63A, C76A, C127A | >2800 |
| C141 | E6K, V11I, C38A, K53A, T63A, C76A, C127A | >2800 |
| C142 | Y1M, E6K, V11I, C38A, K53A, T63A, C76A, C127A | 2.25 |
| C192 | E6K, V11I, C38A, M51G, K53A, T63A, C76A, C127A | >2800 |

### Example 10-IFNγ Induction Cellular Assay

The ability of IL-18 polypeptides provided herein are assessed for ability to induce IFN□ in a cellular assay according to the protocol below.

The NK cell line NK-92 derived from a patient with lymphoma (ATCC® CRL-2407™) is cultured in aMEM medium supplemented with 20% FCS, Glutamax, 25 μM B-mercaptoethanol, and 100 IU/mL of recombinant human IL-2.

On the day of experiment, cells are harvested and washed with aMEM medium without IL-2 and containing 1 ng/mL of recombinant human IL-12. After counting, cells are seeded at 100,000 cells/well in a 384 well titer plate and incubated at 37° C./5% C02. Sixteen 4-fold serial dilutions of IL-18 analytes are prepared in aMEM medium, and 1 ng/mL of IL-12 were added to the NK-92 cells. Final IL-18 analyte concentrations range from 56 nM to 5×10-5 pM.

After incubating the cells for 16-20 hr at 37° C./5% CO2, 5 μL of supernatant is carefully transferred to a 384 microwell OptiPlate. IFNγ levels are measured using a human IFNγ AlphaLISA® Assay Kit. Briefly, 10 μL of 2.5× AlphaLISA Anti-IFNγ acceptor beads and biotinylated antibody anti-IFNγ mix are added to the 5 μL of NK-92 supernatants. The mixtures are incubated for 1 hour at room temperature with shaking. Under subdued light, 2.5 μL of 2× streptavidin (SA) donor beads are pipetted into each well, and the wells are incubated for 30 min at room temperature with shaking. AlphaLISA signals are then measured on an EnSpire™ plate reader using 680 nm and 615 nm as excitation and emission wavelengths, respectively. Half maximal effective concentrations (EC50) are calculated based on a variable slope and four parameter analysis using GraphPad PRISM software.

Act-IL-18 polypeptides provided herein display reduced or eliminated binding ability to stimulate IFNγ compared to WT IL-18 or the IL-18 polypeptide without the artificial terminal moiety. After cleavage, ability to stimulate IFNγ is restored, though may be altered relatve to WT IL-18.

### Example 11-IL-18 Binding Protein Inhibition Cellular Assay

The NK cell line NK-92 derived from a patient with lymphoma (ATCC® CRL-2407™) is cultured in aMEM medium supplemented with 20% FCS-Glutamax, 25 μM B-mercaptoethanol, and 100 IU/mL of recombinant human IL-2.

On the day of experiment, cells are harvested and washed with aMEM medium without IL-2 and containing 1 ng/mL of recombinant human IL-12. After counting, the cells are seeded at 100,000 cells/well in a 384 well titer plate and incubated at 37° C./5% CO2. Sixteen 2-fold serial dilutions of Fc-fused human IL-18 binding protein isoform a (IL-18BPa) are prepared in aMEM medium. 1 ng/mL of IL-12 containing 2 nM of each Act-IL-18 polypeptide variant is added to the NK-92 cells. The final IL-18 analyte concentration is 1 nM, and the final IL-18BPa concentration ranged from 566 nM to 17 pM.

After incubating the cells for 16-20 hr at 37° C./5% CO2, 5 μL of the supernatant is carefully transferred to a 384 microwell OptiPlate. IFNγ levels are measured using a human IFNγ AlphaLISA Assay Kit. Briefly, 10 μL of 2.5× AlphaLISA anti-IFNγ acceptor beads and biotinylated antibody anti-IFNγ mix are added to 5 μL of NK-92 supernatants. The mixtures are incubated for 1 hr at room temperature with shaking. Under subdued light, 2.5 μL of 2×SA donor beads are pipetted in each well and incubated for 30 min at room temperature with shaking. AlphaLISA signals are then measured on an EnSpire™ plate reader using 680 nm and 615 nm as excitation and emission wavelengths, respectively. Half maximal inhibitory concentrations (IC50) are calculated based on a variable slope and four parameter analysis using GraphPad PRISM software.

Act-IL-18 variants of the disclosure are active and able to induce IFNγ secretion in vitro after cleavage of the artificial terminal moiety, but display reduces or no ability to induce IFNγ without cleavage.

The experiments described in Examples 10 and 11 were performed on modified IL-18 polypeptides in order to assess their activities and their suitability for transformation into Act-IL-18 polypeptides.

| SEQ ID NO: or | | | |
|---|---|---|---|
| Composition ID | Sequence modifications | IC₅₀ (nM) | EC₅₀ (nM) |
| 1 | Native sequence | 1.47 | 0.276 |
| 34 | E06K, K53A, S55A | 229 | 0.824 |
| 35 | Y01G, F02A, E06K, M51G, K53A, D54A, S55A, T63A | >55.0 | >55.0 |
| 36 | K53A | 27.3 | 0.444 |
| 37 | S55A | 4.46 | 0.108 |
| 38 | E06K | 7.79 | 0.0567 |
| 39 | E06K, K53A | >703 | 0.0192 |
| 40 | E06K, S55A | 15 | 0.067 |
| 41 | K53A, S55A | 37.3 | 1.58 |
| 42 | E06K, K53A, S55A, T63A | 1060 | 0.144 |
| 43 | E06K, K53A, S55A, Y01G | 27.8 | 6.12 |
| 44 | E06K, K53A, S55A, F02A | NT | >1000 |
| 45 | E06K, K53A, S55A, D54A | NT | 30 |
| 46 | E06K, K53A, S55A, M51G | 0.189 | 7.4 |
| 47 | C38S, C68S, C76S, C127S | 0.444 | 0.115 |
| 48 | C38S, C68S, C76S, C127S, K70C | 0.114 | 0.488 |
| 49 | E06K, K53A, S55A, C38S, C68S, C76S, C127S, K70C | NT | 58.5 |
| 50 | E06K, K53A, T63A | >1000 | 0.0268 |
| 51 | T63A | 0.239 | 0.449 |
| 52 | E06K, T63A | 47.1 | 0.011 |
| 53 | K53A, T63A | 18.2 | 0.155 |
| 54 | E06K, K53A, C38S, C68S, C76S, C127S, K70C | 23.5 | 0.962 |
| 55 | K53A, T63A, C38S, C68S, C76S, C127S, K70C | >1000 | 17.2 |
| 6 | E6K, V11I, C38A, K53A, T63A, C68A, C76A, C127A, | 5.847 | 1.366 |
| | D98C | | |
| 5 | E6K, V11I, C38A, K53A, T63A, C68A, C76A, C127A, | 62.37 | 0.075 |
| | M86C | | |
| 9 | E6K, C38A, K53A, C68A, D98C | 960.8 | 0.069 |
| 4 | E6K, C38A, K53A, C68A, M86C | 396.3 | 0.022 |
| 30 | E6K, V11I, C38A, K53A, T63A, C76A, C127A | 283.6 | 0.026 |
| 62 | E6K, C38A, K53A, C127A | 780.5 | 0.006 |
| 60 | E6K, C38Q, K53A | 653.5 | 0.015 |
| 59 | E6K, C38A, K53A | 146.2 | 0.045 |
| 57 | E6K, C38S, K53A | 1.625 | 0.138 |
| C143 | V11I, C38A, K53A, C76A, C127A | 7.522 | 0.012 |
| C144 | V11I, C38A, K53A, T63A, C76A, C127A | 10.24 | 0.087 |
| C145 | V11I, C38A, K53A, S55A, C76A, C127A | 732.9 | 0.037 |
| C146 | V11I, C38A, M51G, K53A, C76A, C127A | 47.63 | 0.079 |
| C147 | V11I, C38A, K53A, D54A, C76A, C127A | 5.055 | 0.256 |
| C148 | F2A, V11I, C38A, K53A, C76A, C127A | 1.167 | 0.187 |
| C149 | V11I, E31A, C38A, K53A, C76A, C127A | 21.27 | 0.015 |
| C150 | V11I, T34A, C38A, K53A, C76A, C127A | 3.622 | 0.061 |
| C151 | V11I, D35A, C38A, K53A, C76A, C127A | 7.85 | 0.033 |
| C152 | V11I, S36A, C38A, K53A, C76A, C127A | 2.222 | 0.175 |
| C153 | V11I, D37A, C38A, K53A, C76A, C127A | 3.709 | 0.062 |
| C154 | V11I, E31A, D37A, C38A, K53A, C76A, C127A | 3.233 | 0.067 |
| C155 | V11I, C38A, D40A, K53A, C76A, C127A | 0.681 | 0.558 |
| C156 | V11I, C38A, N41A, K53A, C76A, C127A | 6.082 | 0.056 |
| C157 | V11I, C38A, K53A, C76A, C127A, D132A | 3.981 | 0.073 |
| C158 | V11I, C38A, K53A, C76A, G108A, C127A | 1.807 | 0.123 |
| C159 | V11I, C38A, K53A, C76A, H109A, C127A | 3.181 | 0.028 |
| C160 | V11I, C38A, K53A, C76A, D110A, C127A | 1.073 | 0.057 |
| C161 | K8R, V11I, C38A, C76A, Q103E, C127A | 7.292 | 0.061 |
| C162 | K8E, V11I, C38A, C76A, Q103R, C127A | 0.823 | 0.093 |
| C163 | V11I, C38A, C76A, Q103K, C127A | 0.456 | 0.414 |
| C164 | V11I, C38A, S55H, C76A, C127A | 0.885 | 0.176 |
| C165 | V11I, C38A, S55R, C76A, C127A | 0.44 | 0.098 |
| C166 | V11I, C38A, S55T, C76A, C127A | 0.809 | 0.103 |
| C167 | V11I, C38A, C76A, S105I, C127A | 0.176 | 0.098 |
| C168 | V11I, C38A, C76A, S105K, C127A | 5.847 | 1.366 |

### Example 12-Pharmacokinetic and Pharmacodynamic Properties of Act-IL-18 Polypeptide Variants

The pharmacokinetic (PK) and pharmacodynamic (PD) properties of select IL-18 polypeptide variants are measured. Three C57B3L/6 mice are tested per group and per time point. IL-18 variants are applied via single intravenous injections. Mice are divided into four dose groups: 0.5 mg/kg, 0.1 mg/kg, 0.02 mg/kg, 0.004 mg/kg; and four time point groups: 5 min, 6 hr, 24 hr, 48 hr. In a separate experiment, PD and PD is measured as provided for the C57B3L/6 mice, but the healthy mice are replaced with tumor model mice (e.g., mice with MC-38 tumor cell xenografts).

Immune-related PD effects are determined by analyzing cytokine levels in plasma. The following plasma cytokines ae measured: IFNγ, CXCL9, CXCL10, GM-CSF, IL-1a, FasL, and IL-18BP. The activation status of leukocytes is determined by monitoring surface markers: ICOS, PD-1, CD25, CD69, and Fas. Bioanalysis is conducted by detecting the total amount of IL-18 variants (free and IL-18BP-complexed). Corning high-binding half-area plates (Fisher Scientific, Reinach, Switzerland) are coated overnight at 4° C. with 25 μl of anti-IL18 monoclonal antibody (MBL, cat #D043-3, Clone 25-2G) at 2 μg/ml in PBS. Plates are then washed four times with 100 μl of PBS-0.02% Tween20. Plates surfaces are blocked with 25 μl of PBS-0.02% Tween20-1% BSA at 37° C. during 1 h. Plates are then washed four times with 100 μl of PBS-0.02% Tween20. Twenty-five microliters of IL-18 variants (or of mouse plasma) are added in eight-fold serial dilutions starting at 50 nM down to 0.02 nM into PBS-0.02% Tween20-0.1% BSA and incubated at 37° C. during 2 h. Plates are then washed four times with 100 μl of PBS-0.02% Tween20 and 25 μl of biotinylated anti-IL18 monoclonal antibody (MBL, cat #D045-6, Clone 159-12B) at 2 μg/ml in PBS. Plates are incubated during 2 h at 37° C. and are then washed four times with 100 μl of PBS-0.02% Tween20. Twenty-five microliters of Streptavidin—Horseradish peroxidase (#RABHRP3, Merck, Buchs, Switzerland) diluted at 1:500 into PBS-0.02% Tween20-0.1% BSA are added to each well and incubated at Room Temperature during 30 min. Plates are then washed four times with 100 μl of PBS-0.02% Tween20. Fifty microliters of TMB substrate reagent (#CLO7, Merck, Buchs, Switzerland) are added to each well and incubated at 37° C. during 5 min. After 5 min at 37° C., Horseradish peroxidase reaction is stopped by adding 50 μl/well of 0.5M H2SO4 stop solution. ELISA signal is then measured at 450 nm on an EnSpire plate reader from Perkin Elmer (Schwerzenbach, Switzerland).

PK and PD of healthy mice show little activity associated with IL-18 after administration of the Act-IL-18 polypeptide due to the presence of the artificial terminal moiety, though slight effects may be measured due to the presence of endogenous proteases which may cleave the artificial terminal moiety at a low background rate. Distribution of active IL-18 and IL-18 activity is not specific to any tissue.

Conversely, tumor model mice receiving an Act-IL-18 polypeptide with an artificial terminal moiety cleaved preferentially by a tumor associated protease display high local levels of both the active form of the IL-18 and signs of IL-18 activity in and around the tumor microenvironment but little outside of the are in and immediately around the tumor.

### Example 13—PBMC Stimulation Assay

Ability of IL-18 variants to stimulate peripheral blood mononuclear cells (PBMCs) is assessed according to the following protocol.

Isolation of lymphocytes: Blood from Buffy Coats of healthy volunteers is diluted with equal volume of PBS and slowly poured on top of SepMate tube prefilled with 15 mL Histopaque-1077. Tubes are centrifuged for 10 minutes at 1200 g, the top layer is collected and washed 3 times with PBS containing 2% of Fetal Bovine Serum. PBMCs are counted and cryopreserved as aliquots of 20×106 cells.

Cryopreserved PBMCs are thawed and stimulated with gradient of human IL-18 variants ranging from 0.2 pM to 1 μM in RPMI containing 10% Fetal Bovine Serum.

Cytokine production after 24 hr stimulation is measured by Legendplex (Biolegend #740930) on a multicolor flow cytometer. Half maximal effective concentrations (EC50) of IFN□ released in culture supernatant are calculated based on a variable slope and four parameter analysis using GraphPad PRISM software.

Surface expression of FcγRIII on NK cells is measured by flow cytometry (Mouse IgGI clone 3G8) after 72 hr stimulation.

Act-IL-18 variants of the disclosure are active and able to induce IFNγ secretion in vitro after cleavage of the artificial terminal moiety, but display reduces or no ability to induce IFNγ without cleavage.

Although the present disclosure and its advantages have been described in detail, it should be understood that various changes, substitutions and alterations can be made herein without departing from the spirit and scope of the disclosure as defined in the appended claims.

1. An activatable interleukin-18 (Act-IL-18) polypeptide comprising:
an artificial terminal moiety attached to an interleukin-18 (IL-18) polypeptide,
wherein the artificial terminal moiety comprises a specific cleavage site, and
wherein cleavage at the specific cleavage site converts the Act-IL-18 into an active form of the IL-18 polypeptide.

2-4. (canceled)

5. The Act-IL-18 polypeptide of claim 1, wherein the specific cleavage site is specifically cleaved by a protease.

6. (canceled)

7. The Act-IL-18 polypeptide of claim 5, wherein the protease is selected from: kallikrein, thrombin, chymase, carboxypeptidase A, an elastase, proteinase 3 (PR-3), granzyme M, urokinase plasminogen activator (uPA), a calpain, a matrix metalloproteinase (MMP), a disintegrin and metalloproteinase (ADAM), a fibroblast activation protein alpha (FAP), a matriptase, a plasminogen activator, a cathepsin, a caspase, a tryptase, and a tumor cell surface protease.

8-16. (canceled)

17. The Act-IL-18 polypeptide of claim 1, wherein the artificial terminal moiety comprises a peptide, and cleavage of the artificial terminal moiety at the specific cleavage site leaves no amino acid residues of the peptide attached to the IL-18 polypeptide.

18. (canceled)

19. The Act-IL-18 polypeptide of claim 1, wherein the artificial terminal moiety comprises a peptide, and wherein cleavage of the artificial terminal moiety at the specific cleavage site leaves 1, 2, 3, 4, 5, 6, 7, 8, 9, or 10 amino acid residues of the peptide attached to the IL-18 polypeptide.

20-25. (canceled)

26. The Act-IL-18 polypeptide of claim 1, wherein the artificial terminal moiety comprises an IL-18 propeptide or a portion thereof, or a variant thereof, and wherein the artificial terminal moiety is attached to the N-terminus of the IL-18 polypeptide.

27. (canceled)

28. The Act-IL-18 polypeptide of claim 26, wherein the IL-18 propeptide comprises an amino acid sequence having at least 80%, 85%, 90%, 95%, 96%, 97%, 98%, 99%, or 100% sequence identity to the sequence set forth in SEQ ID NO: 89.

29-33. (canceled)

34. The Act-IL-18 polypeptide of claim 1, wherein the artificial terminal moiety comprises a D3 domain of a IL-18 receptor alpha subunit, or a variant thereof, and wherein the artificial terminal moiety is attached to the C-terminus of the IL-18 polypeptide.

35. The Act-IL-18 polypeptide of claim 34, wherein the blocking moiety comprises an amino acid sequence having at least 80%, 85%, 90%, 95%, 96%, 97%, 98%, 99%, or 100% sequence identity to the sequence set forth in SEQ ID NO: 93.

36. The Act-IL-18 polypeptide of claim 34, wherein the artificial terminal moiety comprises a linking peptide between the specific cleavage site and the D3 domain of the IL-18 receptor alpha subunit.

37. The Act-IL-18 polypeptide of claim 1, wherein the active form of the IL-18 polypeptide displays reduced binding to IL-18 binding protein (IL-18BP) compared to WT IL-18.

38. (canceled)

39. The Act-IL-18 polypeptide of claim 1, wherein the active form of the IL-18 polypeptide displays a binding to IL-18R or ability to activate IL-18R which is at most 100-fold less potent relative to WT IL-18.

40-42. (canceled)

43. The Act-IL-18 polypeptide of claim 1, wherein the IL-18 polypeptide comprises a Y01G, F02A, E06K, VIII, C38S, C38A, K53A, D54A, S55A, T63A, C76S, C76A, E85C, M86C, T95C, D98C, C127S, or C127A amino acid substitution, or any combination thereof, wherein residue position numbering of the IL-18 polypeptide is based on SEQ ID NO: 1 as a reference sequence.

44. The Act-IL-18 polypeptide of claim 1, wherein the IL-18 polypeptide comprises E06K and K53A amino acid substitutions, wherein residue position numbering of the IL-18 polypeptide is based on SEQ ID NO: 1 as a reference sequence.

45. The Act-IL-18 polypeptide of claim 44, wherein the IL-18 polypeptide comprises a T63A and VIII amino acid substitution.

46-48. (canceled)

49. The Act IL-18 polypeptide of claim 1, wherein the IL-18 polypeptide comprises the amino acid sequence set forth in SEQ ID NO: 30.

50. (canceled)

51. The Act IL-18 polypeptide of claim 1, wherein the Act IL-18 polypeptide exhibits a half-maximal effective concentration (EC₅₀) for IL-18 receptor signaling activity which is at least 1,000-fold higher, 2,000-fold higher, 5,000-fold higher, 10,000-fold higher, 15,000-fold-higher, or 20,000-fold higher than the activated form of the IL-18 polypeptide.

52. The Act IL-18 polypeptide of claim 34, wherein the Act IL-18 polypeptide exhibits a half-maximal effective concentration (EC₅₀) for IL-18 receptor signaling activity which is from about 10-fold higher to about 100-fold higher than the activated form of the IL-18 polypeptide.

53-66. (canceled)

67. The Act IL-18 polypeptide of claim 1, wherein the terminal residues of the IL-18 polypeptide are substituted such that the artificial terminal moiety is positioned such that the entirety of the artificial terminal moiety is cleaved from the IL-18 polypeptide.

68. The Act IL-18 polypeptide of claim 1, wherein the half-maximal effective concentration (EC₅₀) of the active form of the Act-IL-18 polypeptide's ability to induce IFNγ is less than 10-fold higher than an EC₅₀ (nM) of an IL-18 polypeptide of SEQ ID NO: 1.
